(19) United States
(12) Patent Application Publication
(10) Pub. No.: US 2026/0069127 A1
(43) Pub. Date: Mar. 12, 2026

(54) METHODS AND SYSTEMS FOR DYNAMIC ADJUSTMENT OF VISION TESTS BASED ON HEAD ORIENTATION

(71) Applicant: Zenni Optical, Inc., Novato, CA (US)

(72) Inventors: Steven LEE, Barrington, IL (US); Julia ZHEN, Novato, CA (US); ChyrSong TING, Novato, CA (US); Matthew James GOLINO, Brookhaven, GA (US); Justin Paul DEMPSEY, Ottawa (CA); Jeffrey Joseph FILLINGHAM, Dartmouth (CA)

(21) Appl. No.: 18/827,546

(22) Filed: Sep. 6, 2024

(51) Int. Cl. A61B 3/032; A61B 3/00; G06F 3/01; G06T 11/00

(52) U.S. Cl. CPC A61B 3/032; A61B 3/005; G06F 3/012; G06F 3/013; G06T 11/00; G06T 2200/24; G06T 2210/41

(57) ABSTRACT

A vision test can be adjusted based on a user's head orientation in a virtual environment. An electronic device, such as a head-mounted display, can display a visual stimulus at a target location in a 3D virtual environment. The device can monitor a head orientation of the user wearing the electronic device. The target location of the visual stimulus can be dynamically adjusted based on the head orientation. For example, the target location corresponds to a first orientation. The electronic device can determine whether the head orientation has stabilized at a current orientation a first extended duration of time greater than an orientation threshold and can move the target location of the visual stimulus to follow the current orientation.

### TECHNICAL FIELD

The present disclosure relates to vision test technology. More specifically, methods, systems, devices, and non-statutory computer-readable storage media can be applied to control locations of visual stimuli automatically and dynamically to facilitate vision testing in an extended reality environment.

### BACKGROUND

Traditional methods for visual acuity assessment do not allow for dynamic adjustment of test parameters, leading to less accurate assessments, nor can they be implemented to test eyes and vision at home using household devices in a very environment locked manner.

### SUMMARY

The present disclosure relates to innovative methods and systems that can revolutionize vision care, making vision testing and other exams more accessible and affordable for patients. Additionally, it is contemplated that the principles and features of the present disclosure can be implemented in numerous other applications of display technology, including headsets, heads-up displays, and other microdisplays (e.g., microLED and microOLED) to address challenges and limitations inherent in such products and their uses.

Some implementations of the present disclosure are directed to a method for testing vision. The method includes, at an electronic device including a head-mounted display (HMD), executing a visual assessment application, including generating a user interface corresponding to a three-dimensional (3D) virtual environment; displaying a visual stimulus at a target location in the 3D virtual environment; monitoring a head orientation of a user wearing the electronic device; and dynamically adjusting the target location of the visual stimulus based on the head orientation.

Some implementations of the present disclosure are directed to a method for testing vision. The method includes, at an electronic device including an HMD, executing a visual assessment application, including generating a user interface corresponding to a 3D virtual environment; selecting a first line of sight including a plurality of locations; and displaying a visual stimulus having a fixed stimulus size successively on the plurality of locations in the 3D virtual environment; monitoring a head orientation of a user wearing the electronic device; and dynamically, adjusting a location of the visual stimulus based on the head orientation to keep the visual stimulus on the first line of sight.

Some implementations of the present disclosure are directed to a method for testing vision. The method includes, at an electronic device including an HMD, displaying a video clip including a plurality of image frames, each image frame including a predefined visual stimulus having a respective orientation with respect to a focal point; while displaying the video clip, obtaining eye image data of an eye of a user; collecting or extracting eye response data including a pupil size from the eye image data; determining a spontaneous user response to the video clip based on eye response data; and automatically determining one or more astigmatism parameters based on the spontaneous user response.

Some implementations of the present disclosure are directed to a method for testing vision. The method includes displaying a video clip including a plurality of image frames, each image frame including a predefined visual stimulus having a respective orientation with respect to a focal point, such that the predefined visual stimulus is displayed rotating continuously with respect to the focal point in the video clip; while displaying the video clip, obtaining eye image data of an eye of a user; determining a user response to the video clip based on the eye image data; and automatically determining one or more astigmatism parameters based on the user response.

Some implementations of the present disclosure are directed to a method for testing vision. The method includes, at an electronic device including an HMD, executing a visual assessment application, including generating a user interface corresponding to a 3D virtual environment; displaying a first visual stimulus at a first depth in the user interface, the first depth measured on a first line of sight; displaying the first visual stimulus at a second depth in the user interface, the second depth distinct from the first depth and measured on the first line of sight; obtaining one or more user responses to displaying of the first visual stimulus; and based on the one or more user responses, determining a depth perception level for a user associated with the electronic device.

Some implementations of the present disclosure are directed to a method for testing vision. The method includes, at an electronic device including an HMD, identifying a plurality of lines of sight; for each of the plurality of lines of sight: displaying two visual stimuli at two respective depths surrounding each of a plurality of target depth; and obtaining a user response to displaying of the two stimuli at the two depths surrounding each respective target depth; and based on user responses associated with the respective target depths of the plurality of lines of sight, forming a depth perception map for the user associated with the electronic device.

Some implementations of the present disclosure are directed to a method for testing vision. The method includes, at an electronic device including an HMD, executing a visual assessment application, including generating a user interface corresponding to a 3D virtual environment; displaying a plurality of visual stimuli in the user interface, each visual stimulus is displayed in duplication with respect to a respective target depth; receiving one or more user responses, each user response indicating whether a user perceives a corresponding visual stimulus in duplication at the respective target depth; and based on the one or more user response, determining a depth perception profile of the user, the depth perception profile including a plurality of depth perception levels corresponding to a plurality of target depths.

Some implementations of the present disclosure are directed to a method for testing vision. The method includes, at an electronic device including an HMD, executing a visual assessment application, including generating a user interface corresponding to a 3D virtual environment; displaying a plurality of visual stimuli at a first distance in the 3D virtual environment, the plurality of visual stimuli having a first optotype size and a plurality of first shadings, the first distance and the first optotype size defining a first acuity level; obtaining one or more user responses; based on the one or more user responses, determining a first contrast perception level corresponding to the first acuity level; determining a shading range for a second acuity level based on the first contrast perception level; and determining a plurality of second shadings in the shading range; and displaying the plurality of visual stimuli at a second distance in the 3D virtual environment, the plurality of visual stimuli having a second optotype size and the plurality of second shadings.

Some implementations of the present disclosure are directed to a method for testing vision. The method includes, at an electronic device including an HMD, successively displaying a plurality of visual stimuli corresponding to a plurality of acuity levels in a 3D virtual environment, wherein at each acuity level, the plurality of visual stimuli have a plurality of respective shadings; obtaining a plurality of user responses to the plurality of visual stimuli; for each acuity level, based on the one or more user responses, determining a respective contrast perception level corresponding to the respective acuity level; and generating a contrast profile of a user associated with the electronic device, the contrast profile mapping the respective contrast perception level with respect to the respective acuity level for the plurality of distances.

Some implementations of the present disclosure are directed to a method for testing vision. The method includes, at an electronic device including an HMD, executing a user application configured to enable the vision test; obtaining an instruction to implement a target vision test; in accordance with a determination that the target vision test corresponds to a driver license issuing requirement: loading a VR user interface to create a 3D VR environment; determining an illumination scheme; and displaying a virtual traffic scene on the VR user interface based on the illumination scheme, the virtual traffic scene including a plurality of traffic signs located at a plurality of distances.

Some implementations of the present disclosure are directed to a method for testing vision. The method includes, at an electronic device including an HMD, executing a user application configured to enable the vision test; generating a user interface corresponding to a 3D virtual environment; displaying a plurality of traffic signs at a plurality of distances on a virtual traffic scene; and applying an illumination scheme to the virtual traffic scene.

Some implementations of the present disclosure are directed to a method for testing vision. The method includes, at an electronic device including an HMD, executing a visual assessment application, including generating a user interface corresponding to a 3D virtual environment; identifying a first line of sight of a user associated with the electronic device; selecting a plurality of positions on the first line of sight, wherein each position is located in a respective position range; for each position, displaying an object at a plurality of locations within the respective position range; obtaining a plurality of user responses to displaying the object for each position; and based on the plurality of user responses, determining a depth perception level of the user associated with the first line of sight.

Some implementations of the present disclosure are directed to a method for testing vision. The method includes, at an electronic device including an HMD, executing a visual assessment application, including generating a user interface corresponding to a 3D virtual environment; identifying a plurality of lines of sight of a user associated with the electronic device; generating a depth perception map associated with the plurality of lines of sight; and determining a depth perception range of the user based on the depth perception map.

In some embodiments, a user application can be implemented by an electronic device including an HMD and configured to create a customized extended reality (XR) environment for a user engaged on an XR information platform. Products may be rendered for the user in a three-dimension format in the XR environment, thereby facilitating eyewear selection and fitting. The XR can be an umbrella term encapsulating Augmented Reality (AR), Virtual Reality (VR), Mixed Reality (MR), and everything in between. In this application, any embodiments that apply a VR system can be implemented using an AR or MR system as well.

Additional features and advantages of the subject technology will be set forth in the description below, and in part will be apparent from the description, or may be learned by practice of the subject technology. The advantages of the subject technology will be realized and attained by the structure particularly pointed out in the written description and embodiments hereof as well as the appended drawings.

It is to be understood that both the foregoing general description and the following detailed description are exemplary and explanatory and are intended to provide further explanation of the subject technology.

### BRIEF DESCRIPTION OF THE FIGURES

Various features of illustrative embodiments of the inventions are described below with reference to the drawings. The illustrated embodiments are intended to illustrate, but not to limit, the inventions.

FIG. 1 is an example data processing environment having one or more servers communicatively coupled to one or more computer devices (e.g., a headset device), in accordance with some embodiments.

FIG. 2 is an environment in which a computer device (e.g., a headset device) is applied to facilitate visual assessment or eyewear fitting, in accordance with some embodiments.

FIG. 3 is a block diagram of a computer system (e.g., including a headset device) configured to implement vision assessment or eyewear fitting, in accordance with some embodiments.

FIG. 4 is a block diagram of a machine learning system for training and applying machine learning models (e.g., for glass making), in accordance with some embodiments.

FIG. 5A is a structural diagram of an example neural network applied to process input data in a machine learning model, and FIG. 5B is an example node in the neural network, in accordance with some embodiments.

FIG. 6A is an example tumbling E chart applied in a visual acuity test, and FIGS. 6B-6E are example patterns applied in an astigmatism test, a stereopsis test, a visual field test, and a color blindness test, in accordance with some embodiments.

FIG. 7 is another example visual pattern applied to test visual acuity and astigmatism, in accordance with some embodiments.

FIGS. 8A-8D include four diagrams of example graphical user interfaces rendered to determine a visual acuity score in a virtual environment created by a headset device, in accordance with some embodiments.

FIGS. 9A-9C include three diagrams of example graphical user interfaces rendered to determine a nearsighted or farsighted power in a virtual environment created by a headset device, in accordance with some embodiments.

FIGS. 10A-10F include six diagrams of example graphical user interfaces rendered to determine eye stigmatism in a virtual environment created by a headset device, in accordance with some embodiments.

FIG. 11 is a flow diagram of an example vision test process for determining spherical powers of eyes of a user, in accordance with some embodiments.

FIG. 12A is a diagram of an example field of view including two example lines of sight, in accordance with some embodiments. FIG. 12B is a diagram of an example field of view in which a line of sight 1202 changes with a head orientation, in accordance with some embodiments. FIG. 12C is a diagram of an example field of view in which a line of sight changes with eye positions, in accordance with some embodiments.

FIG. 13 is a flow diagram of an example vision test process for providing a visual stimulus adaptively based on a head orientation, in accordance with some embodiments.

FIG. 14 is a flow diagram of an example vision test process for determining cylinder correction parameters of eyes of a user, in accordance with some embodiments.

FIG. 15A is a cross sectional view of an example human eyeball and an associated prescription, in accordance with some embodiments.

FIGS. 15B-15E are diagrams illustrating four astigmatism schemes applied to assess an astigmatism condition in a 3D virtual environment, in accordance with some embodiments.

FIG. 16 is a flow diagram of an example vision test process for determining one or more astigmatism parameters, in accordance with some embodiments.

FIG. 17 is a flow diagram of an example vision test process for assessing a depth perception level of a user's eyes, in accordance with some embodiments.

FIG. 18 is a diagram of an example horizontal field of view (HFOV) of a user's eyes, in accordance with some embodiments.

FIG. 19 is a flow diagram of an example vision test process for determining a depth perception level of a user, in accordance with some embodiments.

FIG. 20 is a flow diagram of an example vision test process for assessing a stereopsis condition of a user's eyes, in accordance with some embodiments.

FIG. 21 is a flow diagram of an example vision test process for determining a depth perception profile for a user's eyes, in accordance with some embodiments.

FIG. 22 is a flow diagram of an example vision test process for assessing a contrast sensitivity level of a user's eyes, in accordance with some embodiments.

FIG. 23 is a plot of two curves representing example correlations between a size of an object and a contrast level required for a person's eyes to recognize the object, in accordance with some embodiments.

FIG. 24 is a flow diagram of an example vision test process for determining a contrast sensitivity profile of a user, in accordance with some embodiments.

FIG. 25 is a flow diagram of an example vision test process for controlling shadings of visual stimuli in a 3D virtual environment, in accordance with some embodiments.

FIG. 26 is a flow diagram of an example process of selecting one of an AR user interface and a VR user interface to implement a vision test, in accordance with some embodiments.

FIG. 27 is an example traffic scene enabled in a virtual environment for one or more vision tests, in accordance with some embodiments.

FIG. 28 is a flow diagram of an example vision test process for controlling an illumination scheme in a 3D virtual environment, in accordance with some embodiments.

FIG. 29 is a flow diagram of an example vision test process for assessing a contrast sensitivity level of a user's eyes, in accordance with some embodiments.

FIG. 30 is a flow diagram of an example vision test process for assessing a depth perception level of a user's eyes, in accordance with some embodiments.

FIG. 31 is a diagram of two example lines of sights associated with a binocular vision of a user, in accordance with some embodiments.

FIG. 32 is a flow diagram of an example vision test process for determining a depth perception range of a user, in accordance with some embodiments.

### DETAILED DESCRIPTION

It is understood that various configurations of the subject technology will become readily apparent to those skilled in the art from the disclosure, wherein various configurations of the subject technology are shown and described by way of illustration. As will be realized, the subject technology is capable of other and different configurations and its several details are capable of modification in various other respects, all without departing from the scope of the subject technology. Accordingly, the summary, drawings and detailed description are to be regarded as illustrative in nature and not as restrictive.

The detailed description set forth below is intended as a description of various configurations of the subject technology and is not intended to represent the only configurations in which the subject technology may be practiced. The appended drawings are incorporated herein and constitute a part of the detailed description. The detailed description includes specific details for the purpose of providing a thorough understanding of the subject technology. However, it will be apparent to those skilled in the art that the subject technology may be practiced without these specific details. In some instances, well-known structures and components are shown in block diagram form in order to avoid obscuring the concepts of the subject technology. Like components are labeled with identical element numbers for ease of understanding.

Moreover, various aspects of the present disclosure can be implemented in combination with aspects of other virtual-reality technology developed by the present applicant, for example, in copending U.S. Patent App. Nos. 63/560,623 (137034-5002), filed on Mar. 1, 2024, 63/569,095 (137034-5005), filed on Mar. 23, 2024, 63/642,571 (137034-5007), filed on May 3, 2024, 63/642,583 (137034-5009), filed on May 3, 2024, 63/642,593 (137034-5010), filed on May 3, 2024, 63/642,604 (137034-5011), filed on May 3, 2024, 63/644,457 (137034-5012), filed on May 8, 2024, Ser. No. 18/759,641 (137034-5018/1.1), filed on Jun. 28, 2024, and Ser. No. 18/791,203 (137034-5036), filed on Jul. 31, 2024, the entireties of each of which is incorporated herein by reference. Aspects of these copending cases can be implemented in combination with some embodiments disclosed herein, whether in addition to features thereof or as an alternative to a particular feature of an embodiment disclosed herein.

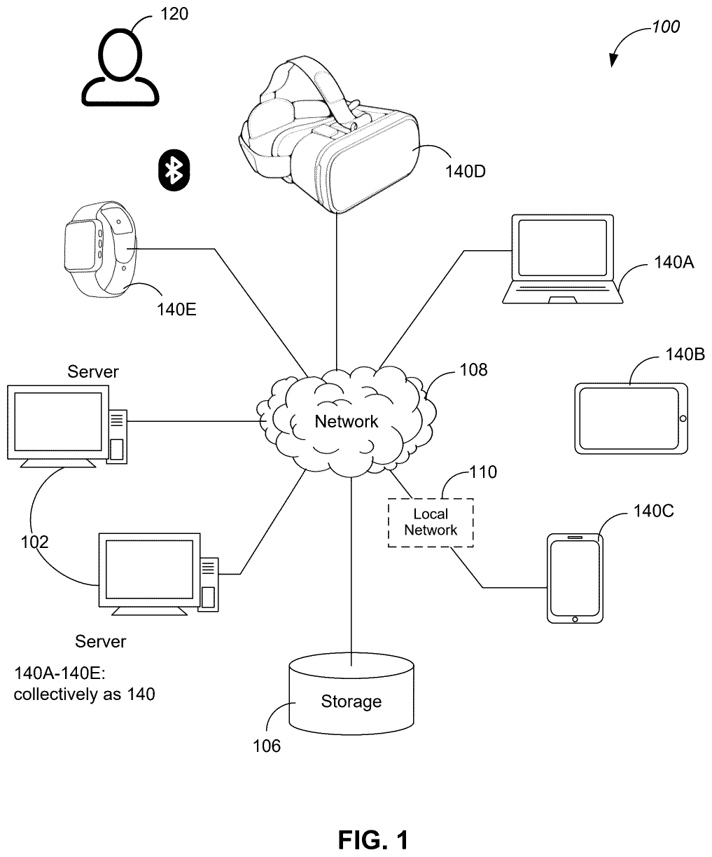

Referring now to the figures, FIG. 1 is an example data processing environment 100 having one or more servers 102 communicatively coupled to one or more computer devices 140 (e.g., a headset device 140D), in accordance with some embodiments. The one or more computer devices 140 are electronic devices having computational capabilities, and may be, for example, desktop computers 140A, tablet computers 140B, mobile phones 140C, or intelligent, multi-sensing, network-connected home devices (e.g., a depth camera, a visible light camera).

In some implementations, the one or more computer devices 140 can include a headset device 140D (e.g., an HMD device 140D) configured to render extended reality content. In some implementations, the one or more computer devices 140 can include a wireless wearable device 140E (e.g., a smart watch, a fitness band) configured to track health data (e.g., heart rate, quality of sleep) and activity data (e.g., steps walked, stairs climbed) of a user wearing the device 140E. Each computer device 140 can collect data or user inputs, executes user applications, and present outputs on its user interface. The collected data or user inputs can be processed locally at the computer device 140 and/or remotely by the server(s) 102. The one or more servers 102 can provide system data (e.g., boot files, operating system images, and user applications) to the computer devices 140, and in some embodiments, processes the data and user inputs received from the computer device(s) 140 when the user applications are executed on the computer devices 140. In some embodiments, the data processing environment 100 can further include a storage 106 for storing data related to the servers 102, computer devices 140, and applications executed on the computer devices 140. For example, storage 106 may store video content, static visual content, and/or audio data.

The one or more servers 102 can enable real time data communication with the computer devices 140 that can be remote from each other or from the one or more servers 102. Further, in some embodiments, the one or more servers 102 can implement data processing tasks that are not completed locally by the computer devices 140. For example, the computer devices 140 can include a game console (e.g., the headset device 140D) that executes an interactive online gaming application (e.g., for visual assessment or eyewear fitting). The game console receives a user instruction and sends it to a server 102 with user data. The server 102 generates a stream of video data based on the user instruction and user data, and provides the stream of video data for display on the game console and other computer devices that can be engaged in the same session with the game console.

The one or more servers 102, one or more computer devices 140, and storage 106 can be communicatively coupled to each other via one or more communication networks 108, which are the medium used to provide communications links between these devices and computers connected together within the data processing environment 100. The one or more communication networks 108 may include connections, such as wire, wireless communication links, or fiber optic cables. Examples of the one or more communication networks 108 include local area networks (LAN), wide area networks (WAN) such as the Internet, or a combination thereof. The one or more communication networks 108 are, optionally, implemented using any known network protocol includes various wired or wireless protocols, such as Ethernet, Universal Serial Bus (USB), FIREWIRE, Long Term Evolution (LTE), Global System for Mobile Communications (GSM), Enhanced Data GSM Environment (EDGE), code division multiple access (CDMA), time division multiple access (TDMA), Bluetooth, Wi-Fi, voice over Internet Protocol (VoIP), Wi-MAX, or any other suitable communication protocol. A connection to the one or more communication networks 108 may be established either directly (e.g., using 1G/4G connectivity to a wireless carrier), or through a network interface 110 (e.g., using a router, switch, gateway, hub, or an intelligent, dedicated whole-home control node), or through any combination thereof. As such, the one or more communication networks 108 can represent the Internet of a worldwide collection of networks and gateways that use the Transmission Control Protocol/Internet Protocol (TCP/IP) suite of protocols to communicate with one another. At the heart of the Internet is a backbone of high-speed data communication lines between major nodes or host computers, consisting of thousands of commercial, governmental, educational and other electronic systems that route data and messages.

In some embodiments, the headset device 140D can be communicatively coupled to a data processing environment 100. The headset device 140D includes one or more cameras (e.g., a visible light camera, a depth camera), a microphone, a speaker, one or more inertial sensors (e.g., gyroscope, accelerometer), and a display. In some embodiments, the camera may capture hand gestures of a user wearing the headset device 140D. In some embodiments, the microphone records ambient sound includes user's voice commands.

In some embodiments, the headset device 140D may be communicatively coupled to one or more servers 102 and enables a centralized vision test management platform with the one or more servers 102. This vision test management platform may aggregate data (e.g., visual stimuli 338, sensor data 342, vision test results 344) from a plurality of user accounts associated with a plurality of users, analyze the aggregated data, and track vision health trends for individual users or user groups. In some embodiments, data may be communicated between a headset device 140D and a server 102 in an encrypted format. In some embodiments, the vision test management platform is coupled to a global health database storing epidemiological data. The vision test management platform can be configured to cross-reference the data collected from its user accounts with the epidemiological data to identify an emerging pattern and a public health concern. For example, a teenager's vision data may be collected and analyzed during an extended duration of time (e.g., 10 years) to identify an individual vision development trend and was cross-referenced with an average vision development trend extracted from the global health database. A doctor can rely on a cross-referencing result to determine whether the individual vision development trend is normal or whether the teenager's eyesight drops faster than average teenagers. As such, various embodiments of the vision test management platform may integrate biometric data and global health analytics and provides a secure, personalized, and interactive environment for vision testing, which can improve precision and user experience of vision assessments and contributes to broader public health monitoring and research initiatives.

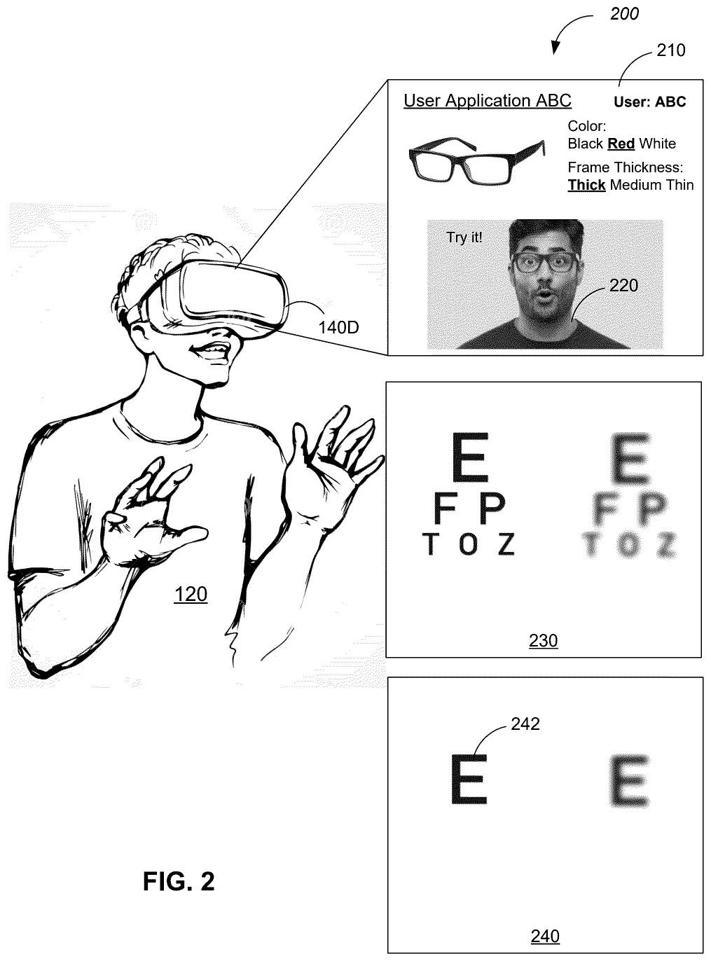

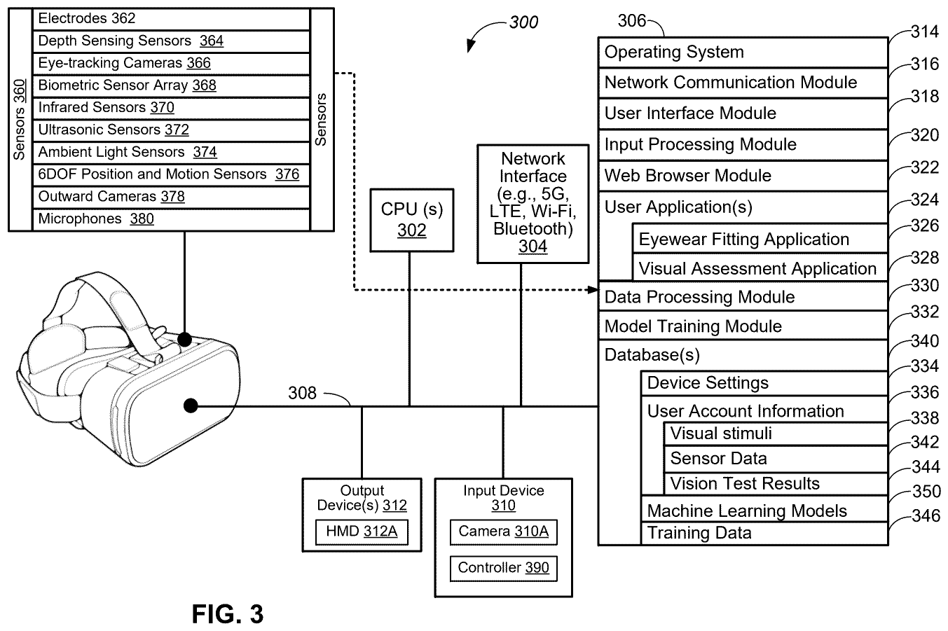

FIG. 2 is an environment 200 in which a computer device 140 (e.g., a headset device 140D) is applied to facilitate visual assessment or eyewear fitting, in accordance with some embodiments. The XR headset device 140D may be communicatively coupled within the data processing environment 100. The XR headset device 140D may include one or more cameras (e.g., a visible light camera, a depth camera), a microphone, a speaker, one or more inertial sensors (e.g., gyroscope, accelerometer), and a display. In some embodiments, the camera may capture hand gestures of a user wearing the XR headset device 140D. In some embodiments, the microphone may record ambient sound includes user's voice commands. The XR headset device 140D may execute a client-side eyewear fitting application 326 or a client-side visual assessment application 328 (FIG. 3) via a user account associated with a user 120 (e.g., an optometrist user, an optician user, a patient user). In some implementations, a computer device 140 (e.g., a mobile phone 140C) distinct from the XR headset device 140D can be used to implement the client-side eyewear fitting application 326 or visual assessment application 328 (FIG. 3).

In some embodiments, a first user interface 210 can be displayed on a computer device 140 (e.g., the headset device 140D) associated with the user 120. In some embodiments, an eyewear can be tried on or displayed as being worn by a 2D or 3D image 220 of the user 120. The server 102 or computer device 140 may receive, from the first user interface 210, a user feedback message indicating an issue, requesting further improvement, or confirming a fit. In some embodiments, a second user interface 230 can be displayed on a computer device 140 associated with the user 120. The second user interface 230 may include a plurality of optotypes (e.g., six optotypes E, F, P, T, O, and Z) having different sizes. In some embodiments, a third user interface 240 can be displayed on a computer device 140 associated with the user 120. The second user interface 230 can display a temporal sequence of optotypes having respective sizes. Each optotype of a corresponding size can be displayed at one time.

FIG. 3 is a block diagram of a computer system 300 (e.g., including a headset device 140D, a server, or a combination thereof) configured to implement vision assessment or eyewear fitting, in accordance with some embodiments. The computer system 300 can include one or more processing units (CPUs) 302, one or more network interfaces 304, memory 306, and one or more communication buses 308 for interconnecting these components (sometimes called a chipset). The computer system 300 may include one or more input devices 310 that facilitate user input, such as a keyboard, a mouse, a voice-command input unit or microphone, a touch screen display, a touch-sensitive input pad, a gesture capturing camera, a controller 390, or other input buttons or controls. Furthermore, in some embodiments, the computer device 140 of the computer system 300 may use a microphone for voice recognition or an eye tracking camera 366 for tracking eyeball movement. In some implementations, the computer device 140 may include one or more optical cameras (e.g., an RGB camera), scanners, or photo sensor units for capturing images. The computer system 300 may also include one or more output devices 312 that enable presentation of user interfaces 210 and media content. The one or more output devices 312 may include one or more speakers and/or one or more visual displays.

The computer system 300 may include one or more sensors 360, which further may include one or more of: a plurality of electrodes 362, one or more depth sensing sensors 364, one or more eye tracking cameras 366, a biometric sensor array 368, one or more infrared sensors 370, one or more ultrasonic sensors 372, one or more ambient sensors 374, one or more motion sensors (e.g., six degree of freedom (6DOF) position and motion sensors 376), one or more outward camera 378, and one or more microphones 380. It is noted that the one or more sensors 360 can also be included in the input device 310 and used to collect data to the computer system 300.

Memory 306 may include high-speed random-access memory, such as DRAM, SRAM, DDR RAM, or other random-access solid state memory devices; and, optionally, may include non-volatile memory, such as one or more magnetic disk storage devices, one or more optical disk storage devices, one or more flash memory devices, or one or more other non-volatile solid state storage devices. Memory 306, optionally, may include one or more storage devices remotely located from one or more processing units 302. Memory 306, or alternatively the non-volatile memory within memory 306, may include a non-transitory computer readable storage medium. In some implementations, memory 306, or the non-transitory computer readable storage medium of memory 306, may store the following programs, modules, and data structures, or a subset or superset thereof:
Operating system 314 including procedures for handling various basic system services and for performing hardware dependent tasks;
Network communication module 316 for connecting each server 102 or computer device 140 to other devices (e.g., server 102, computer device 140, or storage 106) via one or more network interfaces 304 (wired or wireless) and one or more communication networks 108, such as the Internet, other wide area networks, local area networks, metropolitan area networks, and so on;
User interface module 318 for enabling presentation of information (e.g., a graphical user interface for application(s) 324, widgets, websites and web pages thereof, and/or games, audio and/or video content, text, etc.) at each computer device 140 via one or more output devices 312 (e.g., displays, speakers, etc.);
Input processing module 320 for detecting one or more user inputs or interactions from one of the one or more input devices 310 and interpreting the detected input or interaction;
Web browser module 322 for navigating, requesting (e.g., via HTTP), and displaying websites and web pages thereof may include a web interface for logging into a user account associated with a computer device 140 or another electronic device, controlling the computer device if associated with the user account, and editing and reviewing settings and data that are associated with the user account;
One or more user applications 324 for execution by the computer system 300 (e.g., games, social network applications, smart home applications, extended reality application, and/or other web or non-web-based applications for controlling another electronic device and reviewing data captured by such devices), where in some embodiments, an eyewear fitting application 326 can be executed to implement eyewear fitting, and has a plurality of user accounts associated with a plurality of users 120 (e.g., technician users and eyewear users), and in some embodiments, a visual assessment application 328 can be executed to evaluate eyesight of a patient user, and has a plurality of user accounts associated with a plurality of users 120 (e.g., an optometrist user, a patient user);
Data processing module 330 for processing data associated with the user applications 324, e.g., using machine learning models 350;
Model training Module 332 for obtaining training data 346 and training machine learning models 350; and
One or more databases 340 for storing at least data including one or more of:
   Device settings 334 including common device settings (e.g., service tier, device model, storage capacity, processing capabilities, communication capabilities, etc.) of the computer system 300;
   User account information 336 for the one or more user applications 324, e.g., user names, security questions, account history data, user preferences, and predefined account settings, where in some embodiments, the user account information 336 may include facial measurements and one or more virtual fitting parameters associated with associated with a user account of an eye fitting application 326, and in some embodiments, the user account information 336 may include visual stimuli 338, sensor data 342, and vision test results 344 associated with a user account of a visual assessment application 328; and
   Machine learning models 350 including parameters (e.g., weights, biases) used to implement vision test or select eyewear for eyewear users.

Each of the above identified elements may be stored in one or more of the previously mentioned memory devices, and corresponds to a set of instructions for performing a function described above. The above identified modules or programs (i.e., sets of instructions) need not be implemented as separate software programs, procedures, modules or data structures, and thus various subsets of these modules may be combined or otherwise re-arranged in some embodiments. In some embodiments, memory 306, optionally, stores a subset of the modules and data structures identified above. Furthermore, memory 306, optionally, stores additional modules and data structures not described above.

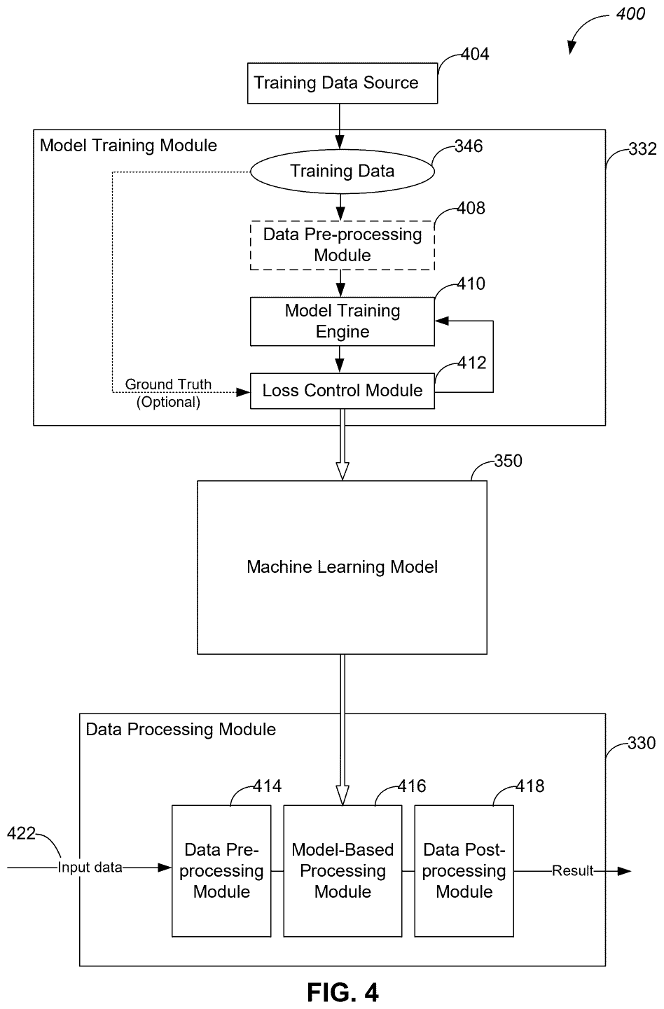

FIG. 4 is a block diagram of a machine learning system 400 for training and applying machine learning models 350 (e.g., for glass making), in accordance with some embodiments. The machine learning system 400 may include a model training module 332 establishing one or more machine learning models 350 and a data processing module 330 for processing input data 422 using the machine learning model 350. In some embodiments, both the model training module 332 and the data processing module 330 may be located within a computer device 140 (e.g., a VR headset), while a training data source 404 provides training data 346 to the computer device 140. In some embodiments, the training data source 404 may include the data obtained from the computer device 140 itself, from a server 102, from storage 106, or from another electronic device or computer device 140. Alternatively, in some embodiments, the model training module 332 may be located at a server 102, and the data processing module 330 may be located in a computer device 140. The server 102 can train the machine learning model 350 and provide the trained models 350 to the computer device 140 to process real time input data 422 detected by the computer device 140. In some embodiments, the training data 346 provided by the training data source 404 may include a standard dataset widely used to train machine learning models 350. The input data 422 further may include sensor data. Further, in some embodiments, a subset of the training data 346 may be modified to augment the training data 346. The subset of modified training data may be used in place of or jointly with the subset of training data 346 to train the machine learning models 350.

In some embodiments, the model training module 332 may include a model training engine 410, and a loss control module 412. Each machine learning model 350 may be trained by the model training engine 410 to process corresponding input data 422 and implement a respective task. Specifically, the model training engine 410 may receive the training data 346 corresponding to a machine learning model 350 to be trained and process the training data to build the machine learning model 350. In some embodiments, during this process, the loss control module 412 can monitor a loss function comparing the output associated with the respective training data item to a ground truth of the respective training data item. In these embodiments, the model training engine 410 may modify the machine learning models 350 to reduce the loss, until the loss function satisfies a loss criteria (e.g., a comparison result of the loss function is minimized or reduced below a loss threshold). The machine learning models 350 may thereby be trained and provided to the data processing module 330 of a computer device 140 to process real time input data 422 from the computer device 140.

In some embodiments, the model training module 402 may further include a data pre-processing module 408 configured to pre-process the training data 346 before the training data 346 is used by the model training engine 410 to train a machine learning model 350. For example, an image pre-processing module 408 is configured to format patients'eye images in the training data 346 into a predefined image format. For example, the preprocessing module 408 may normalize the images to a fixed size, resolution, or contrast level. In another example, an image pre-processing module 408 extracts a region of interest (ROI) corresponding to an eye area.

In some embodiments, the model training module 332 can use supervised learning in which the training data 346 may be labelled and include a desired output for each training data item (also called the ground truth, in some embodiments). In some embodiments, the desirable output may be labelled manually by people or automatically by the model training model 332 before training. In some embodiments, the model training module 332 may use unsupervised learning in which the training data 346 is not labelled. The model training module 332 is configured to identify previously undetected patterns in the training data 346 without pre-existing labels and with little or no human supervision. Additionally, in some embodiments, the model training module 332 may use partially supervised learning in which the training data is partially labelled.

In some embodiments, the data processing module 330 may include a data pre-processing module 414, a model-based processing module 416, and a data post-processing module 418. The data pre-processing modules 414 may pre-process input data 422 based on the type of the input data 422. In some embodiments, functions of the data pre-processing modules 414 are consistent with those of the pre-processing module 408. The data pre-processing modules 414 can convert the input data 422 into a predefined data format that is suitable for the inputs of the model-based processing module 416. The model-based processing module 416 may apply the trained machine learning model 350 provided by the model training module 332 to process the pre-processed input data 422. In some embodiments, the model-based processing module 416 can also monitor an error indicator to determine whether the input data 422 has been properly processed in the machine learning model 350. In some embodiments, the processed input data may be further processed by the data post-processing module 418 to create a preferred format or to provide additional information that can be derived from the processed input data. The data processing module 330 may use the processed input data to make eyewear glasses for a patient user.

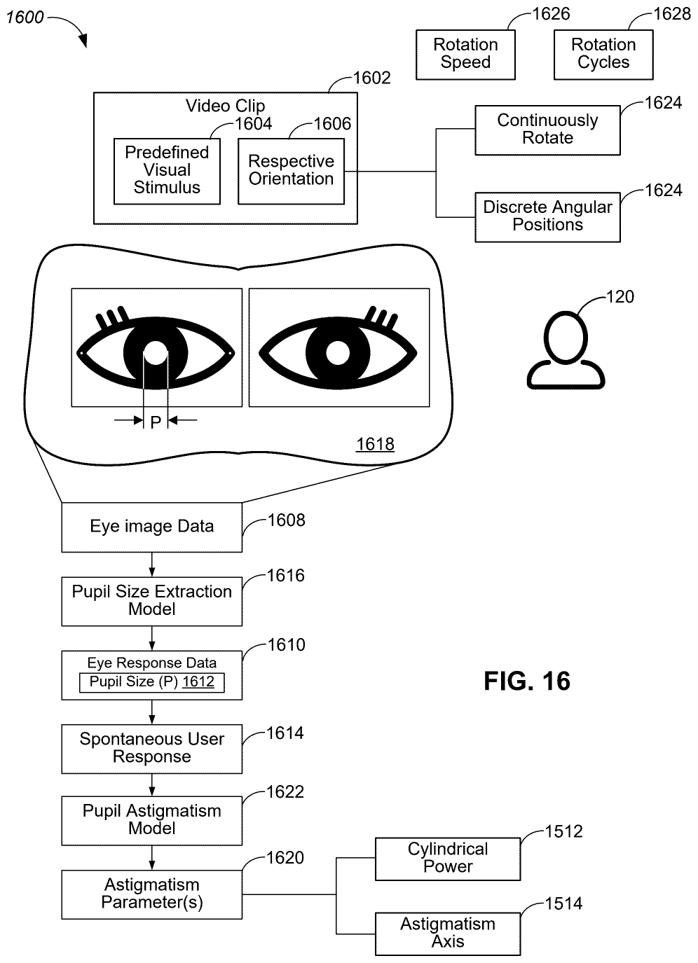

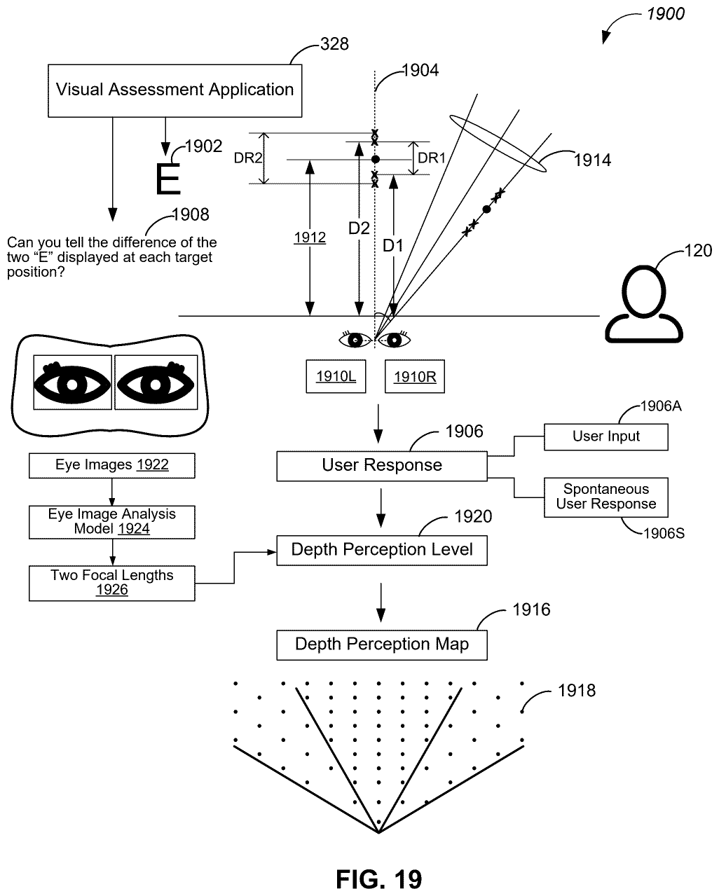

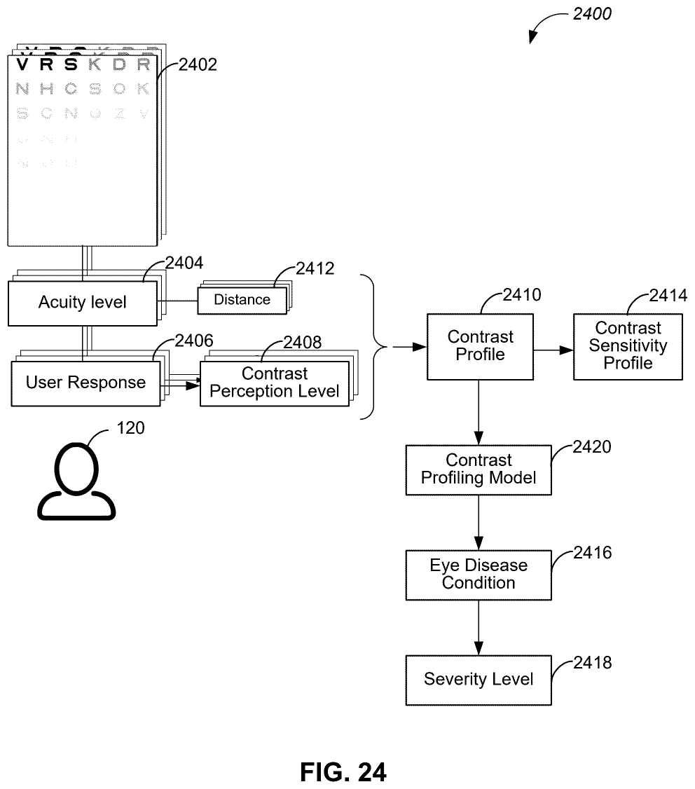

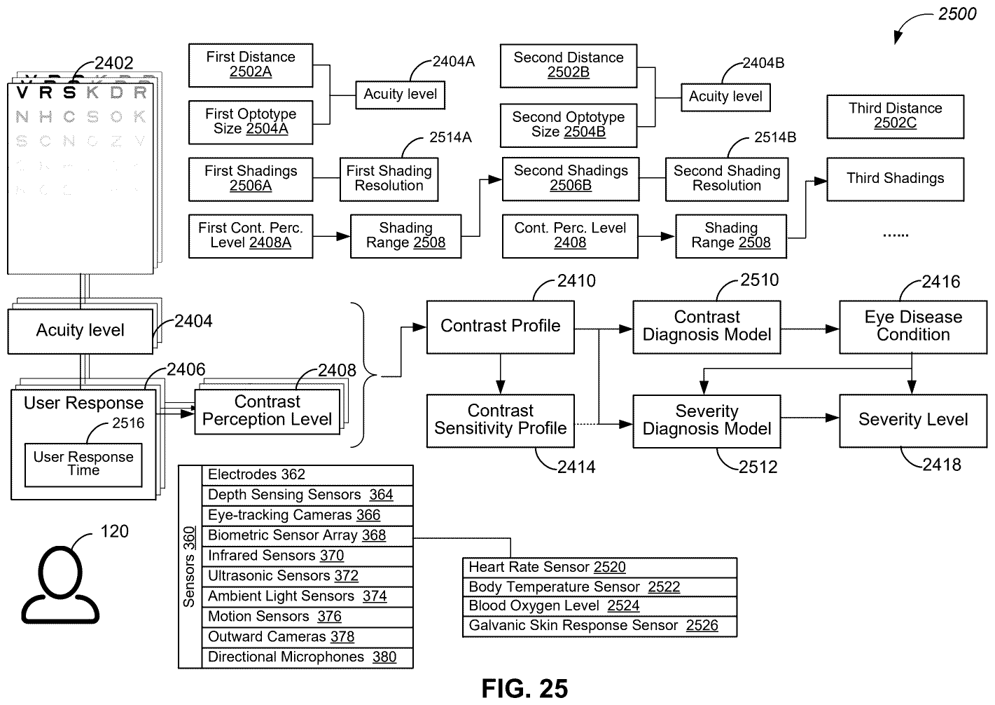

Examples of the machine learning model 350 include, but are not limited to, a pupil size extraction model (FIG. 16), a pupil astigmatism model 1622 (FIG. 16), a focus extraction model 1924 (FIG. 19), a contrast profiling model 2420 (FIG. 24), a contrast diagnosis model 2510 (FIG. 25), and a severity diagnosis model 2512 (FIG. 25).

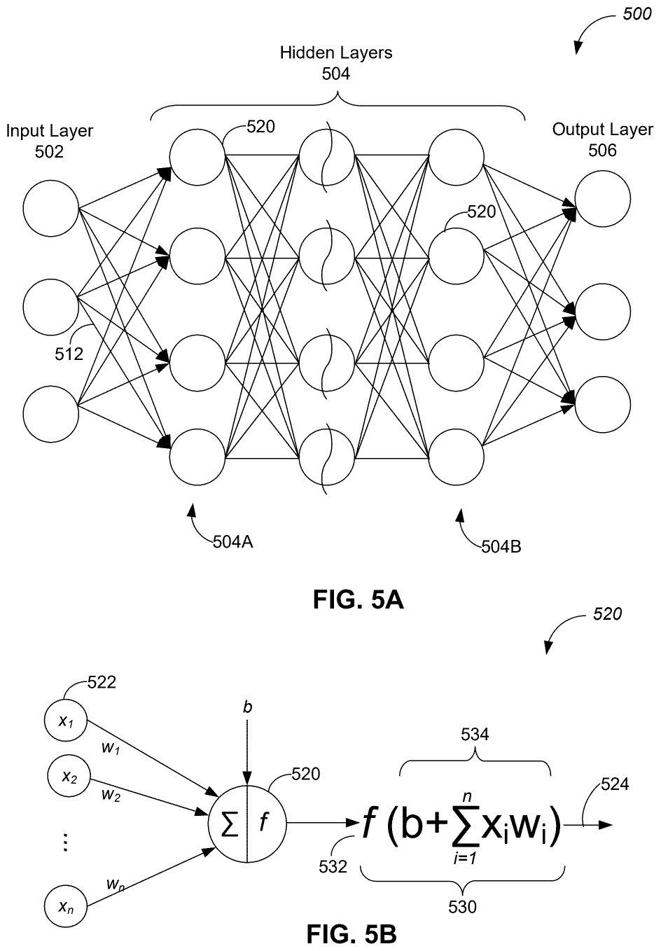

FIG. 5A is a structural diagram of an example neural network 500 applied to process input data in a machine learning model 350, in accordance with some embodiments. Further, FIG. 5B is an example node 520 in the neural network 500, in accordance with some embodiments. It should be noted that this description is used as an example only, and other types or configurations may be used to implement the embodiments described herein. The machine learning model 350 may be established based on the neural network 500. A corresponding model-based processing module 416 may apply the machine learning model 350 including the neural network 500 to process input data 422 that has been converted to a predefined data format. The neural network 500 may include a collection of nodes 520 that may be connected by links 512. Each node 520 may receive one or more node inputs 522 and applies a propagation function 530 to generate a node output 524 from the one or more node inputs. As the node output 524 is provided via one or more links 512 to one or more other nodes 520, a weight w associated with each link 512 may be applied to the node output 524. Likewise, the one or more node inputs 522 may be combined based on corresponding weights w₁, w₂, w₃, and w₄ according to the propagation function 530. In an example, the propagation function 530 is computed by applying a non-linear activation function 532 to a linear weighted combination 534 of the one or more node inputs 522.

The collection of nodes 520 may be organized into layers in the neural network 500. In general, the layers may include an input layer 502 for receiving inputs, an output layer 506 for providing outputs, and one or more hidden layers 504 (e.g., layers 504A and 504B) between the input layer 502 and the output layer 506. A deep neural network has more than one hidden layer 504 between the input layer 502 and the output layer 506. In the neural network 500, each layer may only be connected with its immediately preceding and/or immediately following layer. In some embodiments, a layer may be a fully connected layer because each node in the layer is connected to every node in its immediately following layer. In some embodiments, a hidden layer 504 may include two or more nodes that may be connected to the same node in its immediately following layer for down sampling or pooling the two or more nodes. In particular, max pooling may use a maximum value of the two or more nodes in the layer for generating the node of the immediately following layer.

In some embodiments, a convolutional neural network (CNN) may be applied in a machine learning model 350 to process input data. The CNN employs convolution operations and belongs to a class of deep neural networks. The hidden layers 504 of the CNN include convolutional layers. Each node in a convolutional layer may receive inputs from a receptive area associated with a previous layer (e.g., nine nodes). Each convolution layer may use a kernel to combine pixels in a respective area to generate outputs. For example, the kernel may be to a 3×3 matrix including weights applied to combine the pixels in the respective area surrounding each pixel. Video or image data can be pre-processed to a predefined video/image format corresponding to the inputs of the CNN. In some embodiments, the pre-processed video or image data may abstracted by the CNN layers to form a respective feature map. In this way, video and image data can be processed by the CNN for video and image recognition or object detection.

In some embodiments, a recurrent neural network (RNN) is applied in the machine learning model 350 to process input data 422. Nodes in successive layers of the RNN follow a temporal sequence, such that the RNN exhibits a temporal dynamic behavior. In an example, each node 520 of the RNN has a time-varying real-valued activation. It is noted that in some embodiments, two or more types of input data may be processed by the data processing module 330, and two or more types of neural networks (e.g., both a CNN and an RNN) may be applied in the same machine learning model 350 to process the input data jointly.

The training process is a process for calibrating all of the weights wᵢ for each layer of the neural network 500 using training data 346 that is provided in the input layer 502. The training process typically may include two steps, forward propagation and backward propagation, which may be repeated multiple times until a predefined convergence condition is satisfied. In the forward propagation, the set of weights for different layers may be applied to the input data and intermediate results from the previous layers. In the backward propagation, a margin of error of the output (e.g., a loss function) is measured (e.g., by a loss control module 412), and the weights may be adjusted accordingly to decrease the error. The activation function 532 can be linear, rectified linear, sigmoidal, hyperbolic tangent, or other types. In some embodiments, a network bias term b may be added to the sum of the weighted outputs 534 from the previous layer before the activation function 532 is applied. The network bias b may provide a perturbation that helps the neural network 500 avoid over fitting the training data. In some embodiments, the result of the training may include a network bias parameter b for each layer.

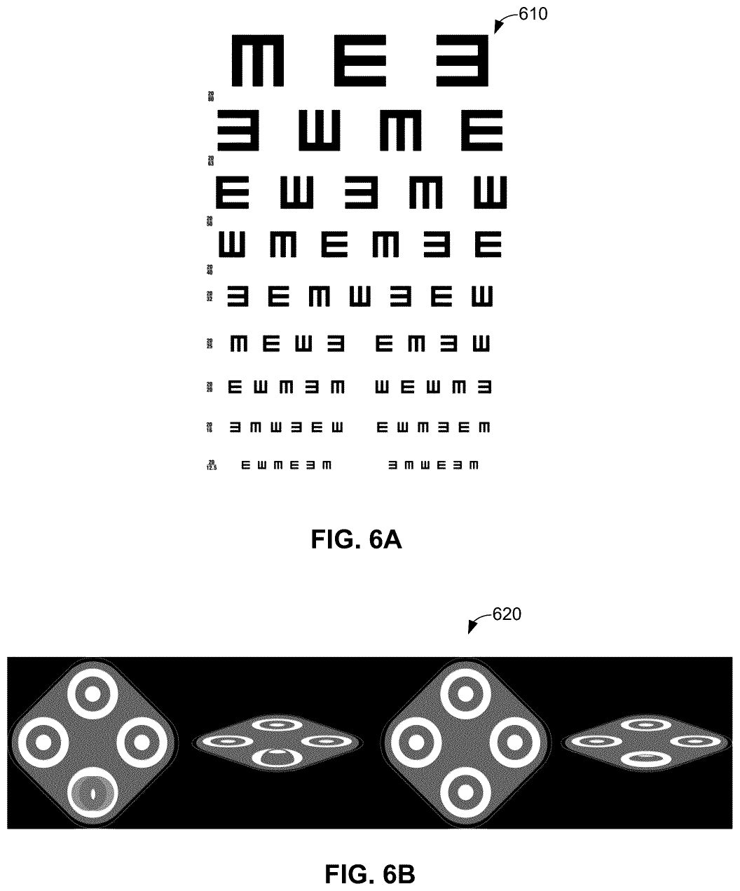

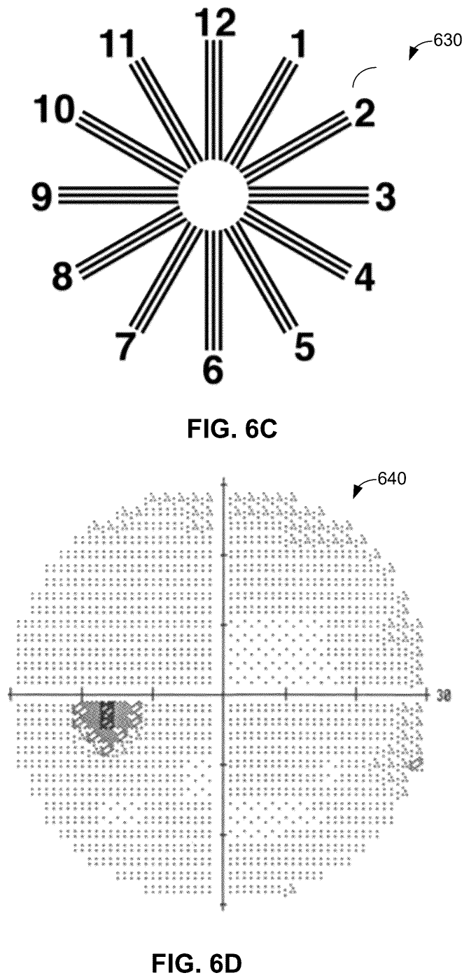

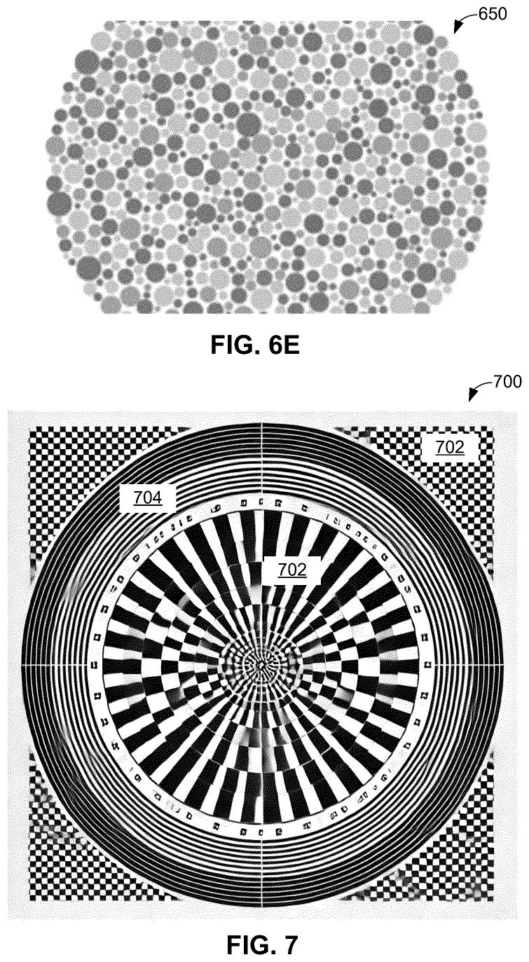

In some embodiments of the present disclosure, a vision test is implemented in a headset device 140D configured to display a user interface creating a three-dimensional (3D) virtual environment. Examples of a vision test implemented in the 3D virtual environment include, but are not limited to a visual acuity test, a visual field test, a visual depth test, a color blindness test, a retinoscopy, a test for stereopsis, a refraction test, an astigmatism test, and a contact lens exam. FIG. 6A is an example tumbling E chart 610 applied in a visual acuity test, in accordance with some embodiments. FIGS. 6B, 6C, 6D, and 6E are example patterns 620, 630, 640, and 650 applied in a stereopsis test, an astigmatism test, a visual field test, and a color blindness test, in accordance with some embodiments.

FIG. 7 is another example visual pattern 700 applied to test visual acuity and astigmatism, in accordance with some embodiments. The visual pattern 700 integrates a grid pattern 702 and concentric rings 704. The grid pattern 702 may include evenly spaced horizontal and vertical lines, creating a checkerboard pattern. The grid pattern 702 may be configured to identify distortions in straight lines, which can indicate issues with visual acuity and astigmatism. The concentric rings 704 may expand outward from a center of the visual pattern 700 and can assist in detecting radial distortions, which are common indicators of astigmatism. The visual pattern 700 may be depicted in high-contrast black and white, which ensures maximum clarity and reduces the potential for color-related distortions, making it easier to detect any visual impairment or defect.

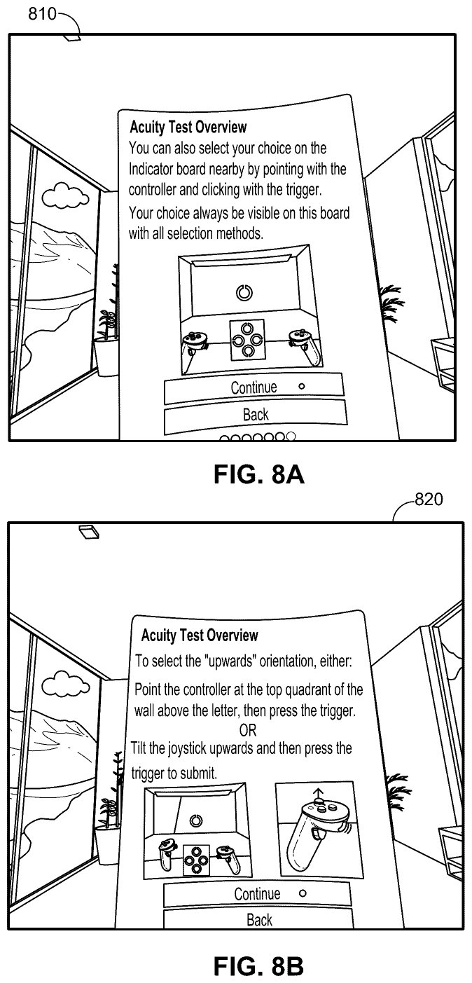

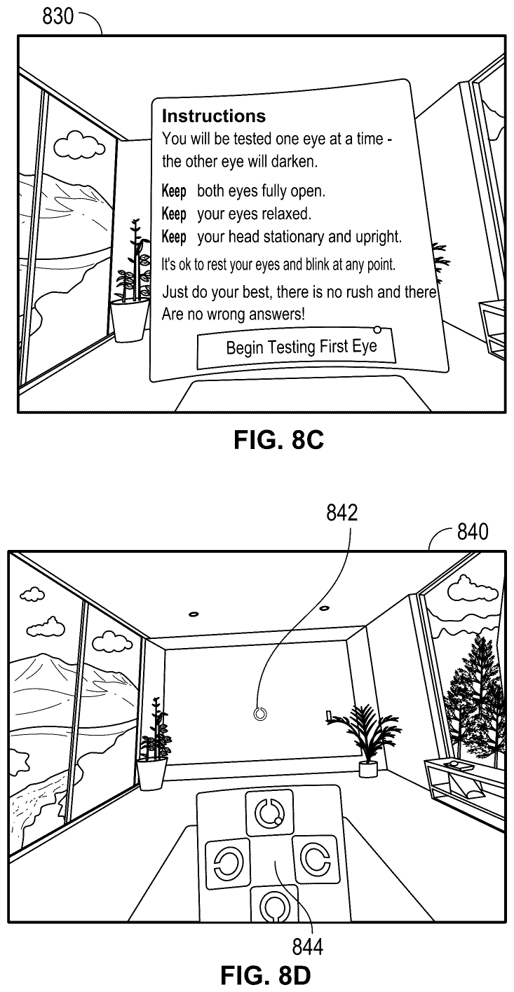

FIGS. 8A-8D include four diagrams of example graphical user interfaces 810, 820, 830, and 840 rendered to determine a visual acuity score in a virtual environment created by a headset device 140D, in accordance with some embodiments. The user interface 810 may display an information page including instructions on controlling a headset device 140D to select one of a plurality of optotype candidates to match a target optotype displayed in the virtual environment. The user interface 820 may display an information page including two optional ways of using a controller 390 (FIG. 3) to select the one of the plurality of optotype candidates. The user interface 830 may display an information page including general guidelines on a visual acuity assessment process. The user interface 840 may display an optotype 842 that is projected on a screen that has a first distance L1 from a user's position in the virtual environment. In a second distance L2 near the user, a selection panel 844 including a plurality of optotype candidates may be displayed, prompting the user to select one of the optotype candidates that matches the optotype 842. In some embodiments, in response to a user selection of the one of the optotype candidates, the optotype 842 displayed in the first distance L1 may be updated with a new optotype 842. Further, in some embodiments, the new optotype 842 may spin at a fast rate for a shortened duration of time (e.g., 2 seconds), before it settles in place of the original optotype 842. In an example, the optotype 842 may spin and gradually shrink in size during the shortened duration of time.

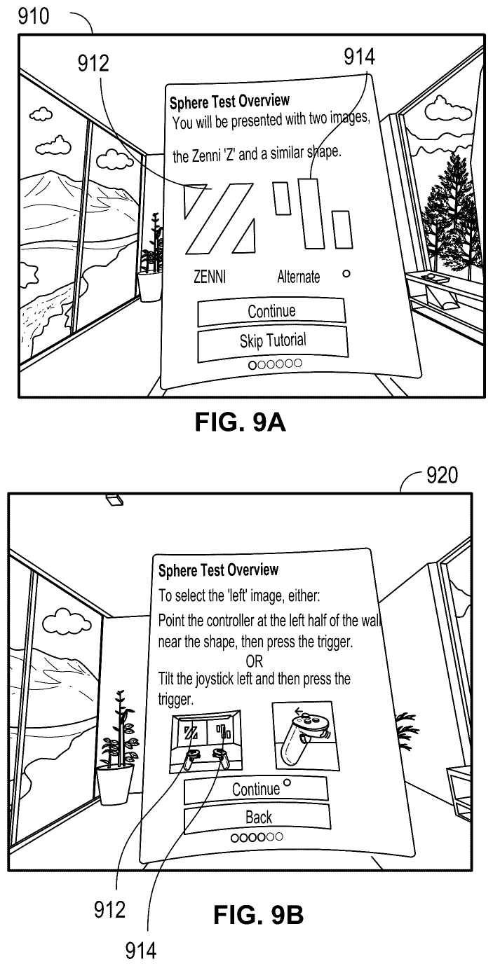

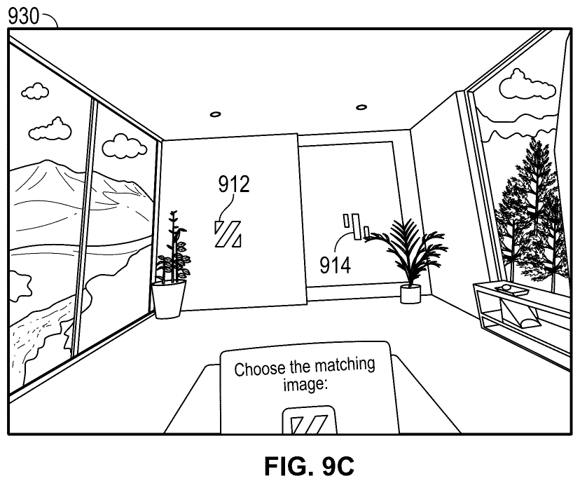

FIGS. 9A-9C include three diagrams of example graphical user interfaces 910, 920, and 930 rendered to determine a nearsighted or farsighted power in a virtual environment created by a headset device 140D, in accordance with some embodiments. The user interface 910 may display an information page explaining that two target optotypes 912 and 914 may be displayed in the virtual environment. The user interface 920 may display an information page including two optional ways of using a controller 390 (FIG. 3) to select one of the two target optotypes 912 and 914. The user interface 930 may display two target optotypes 912 and 914 that may be projected on a screen that has a first distance L1 from a user's position in the virtual environment. In this example, the target optotype 912 located on the left is highlighted (e.g., by being displayed in a colored background). In a second distance L2 near the user, a confirmation panel 932 may be displayed, prompting the user to select one of the two target optotypes 912 and 914. In some embodiments, in response to a user selection of the one of the two target optotypes 912 and 914, the two target optotypes 912 and 914 displayed in the first distance L1 may be updated with a new pair of two target optotypes 912 and 914. Further, in some embodiments, each optotype 912 or 914 may spin at a fast rate for a shortened duration of time (e.g., 2 seconds), before it settles in place of the original optotype 912 or 914. In an example, the optotype 912 or 914 may spin and gradually shrink in size during the shortened duration of time.

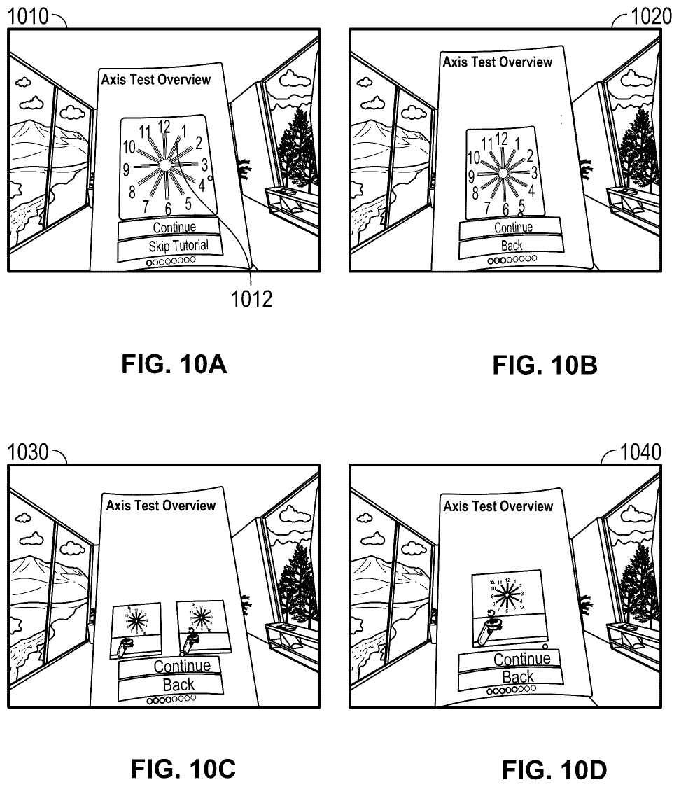

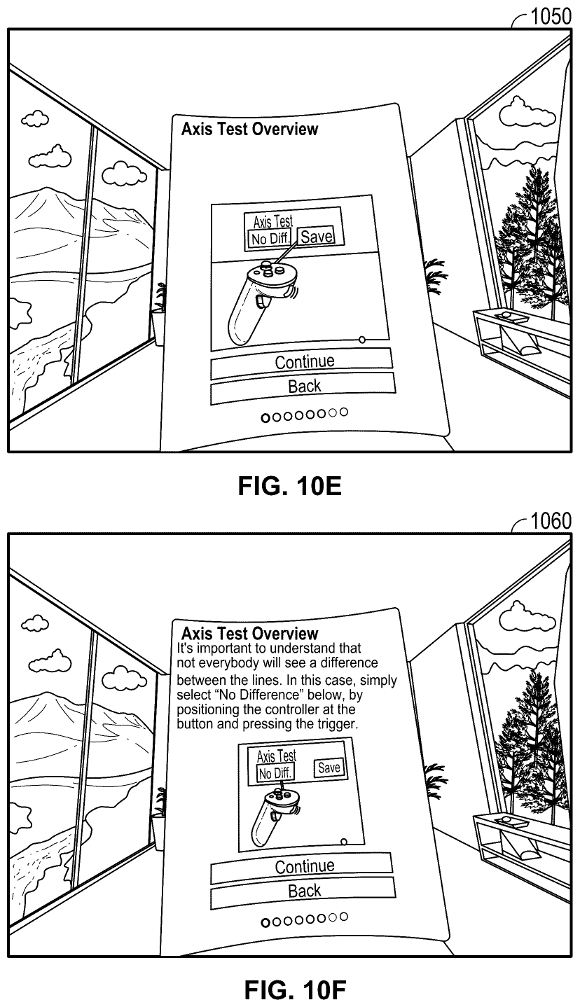

FIGS. 10A-10F include six diagrams of example graphical user interfaces 1010, 1020, 1030, 1040, 1050, and 1060 rendered to determine eye stigmatism in a virtual environment created by a headset device 140D, in accordance with some embodiments. The user interface 1010 may display an information page explaining that a clock diagram of converging numbered lines 1012 (which is a type of optotype) is displayed in the virtual environment. For example, the user interface 1010 may include a message, e.g., “You will be presented with a clock diagram of converging numbered lines.” The user interface 1020 may display an information page explaining what is selected on the clock diagram of converging numbered lines 1012 displayed in the virtual environment. For example, the user interface 1010 may include a message, e.g., “Your task is to identify if any of these sets of lines appear clearer, crisper, or darker than other.” The user interface 1030 may display an information page including two optional ways of using a controller 390 (FIG. 3) to select lines on the clock diagram of converging numbered lines 1012. For example, the user interface 1010 may include a message, e.g., “Make a selection by either pointing the controller 390 at the lines on the clock, then pressing the trigger and Rotating the joystick to move the indicator arrows around the clock.” The user interface 1040 may display an information page illustrating an embodiment having equally clear lines on the clock diagram of converging numbered lines 1012. For example, the user interface 1010 may include a message, e.g., “If two sets of neighboring lines seem to both stand out as equally clear, you can move the indicator arrows to a halfway point between those lines.”

Referring to FIG. 10E, the user interface 1050 may display an information page including an instruction using the controller 390 to submit a selection. For example, the user interface 1010 may include a message, e.g., “After selecting a set of lines, submit your choice with the ‘Done’ button below by pointing to the controller 390 at the button and pressing the trigger.” Further, referring to FIG. 10F, the user interface 1060 may display an information page including an instruction using the controller 390 to indicate that no difference is observed on the clock diagram of converging numbered lines 1012. For example, the user interface 1010 may include a message, e.g., “It's important to understand that not everybody will see a difference between the lines and In this case, simply select ‘No Difference’ below, by positioning the controller at the button and pressing the trigger.

### Head Orientation Based Vision Tests

Some implementations of this application include a VR-based computer system 300 configured to measure spherical powers of eyes of a patient through interactive visual challenges. The computer system 300 may utilize a high-resolution VR headset that may be equipped with eye-tracking sensors (e.g., eye-tracking cameras 366 in FIG. 3) and specialized software algorithms to create a series of engaging visual tasks. Users may wear the VR headset and participate in various interactive challenges that require focusing on objects at different distances and under varying visual conditions. The eye-tracking sensors may monitor the user's focus adjustments and visual acuity, while the software analyzes the user responses to determine the spherical power of the user's eyes.

In some embodiments, the VR-based computer system 300 may incorporate a range of visual tasks, such as reading text at different distances, identifying objects in a 3D space, and following moving targets. These tasks may be configured to dynamically assess the user's ability to focus and refocus, providing data on the eye's refractive error. A user application 324 (e.g., visual assessment application 328 in FIG. 3) may process the data in real time and calculate the spherical powers needed to correct the user's vision. Results may be compiled into a detailed report that provides measurement of the spherical power of each eye or recommendations for corrective lenses. The computer system 300 offers a non-invasive, engaging, and accurate approach to determine refractive errors.

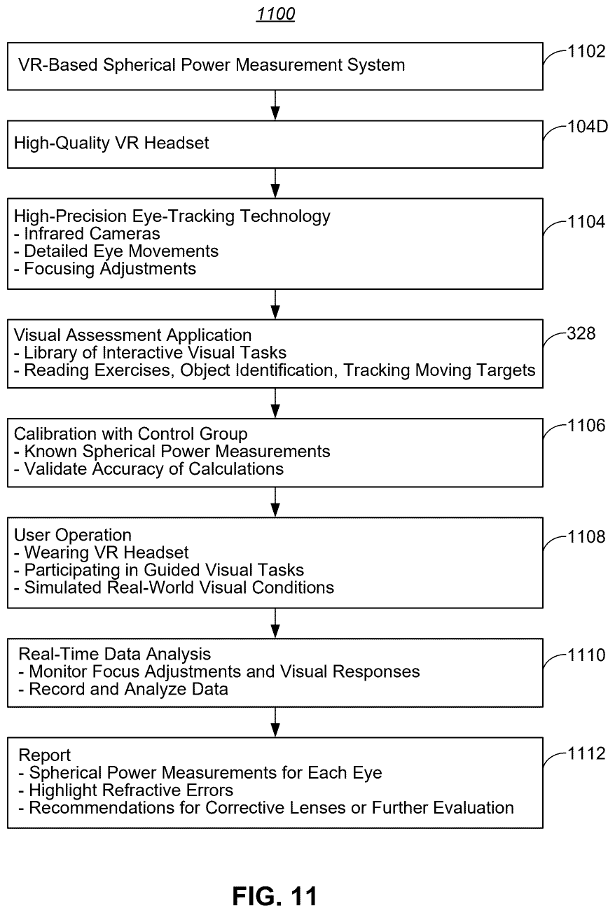

FIG. 11 is a flow diagram of an example vision test process 1100 for determining spherical powers of eyes of a user, in accordance with some embodiments. The VR-based computer system 300 may be configured to enable a spherical power measurement system 1102. The computer system 300 may include a VR headset 104D that includes an eye-tracking camera 366 (FIG. 3). The eye-tracking technology 1104 may include an infrared camera (e.g., camera 366) configured to capture eye movements and focusing adjustments. In some embodiments, when a visual assessment application 328 is executed, a library of interactive visual tasks is applied to test different aspects of focusing and visual acuity. Examples of the interactive visual tasks include, but are not limited to, reading exercises, object identification, and tracking moving targets. The interactive visual tasks may be implemented in a visual assessment application 328 in a three-dimensional virtual environment to simulate real-world visual conditions.

In some embodiments, when hardware components and software modules may be integrated to form the spherical power measurement system 1102, the VR-based computer system 300 may be calibrated (operation 1106) using a control group of individuals with known spherical power measurements. Users can operate (operation 1108) the calibrated computer system 300 by wearing the VR headset and participating in the guided visual tasks. The eye-tracking camera 366 may monitor focus adjustments and visual responses of a user's eyes to interactive challenges. Image or video data recorded by the camera 366 may be analyzed (operation 1110) in real time by the software modules (e.g., visual assessment application 328, data processing module 330 in FIG. 3). In some implementations, the user may receive a report 1112 outlining spherical power measurements for each eye, and the report may indicate refractive errors and provide recommendations for corrective lenses or further ophthalmic evaluation. By these means, the computer system 300 may offer a precise, non-invasive, and user-friendly method for measuring spherical power parameters, representing a significant advancement over traditional refractive assessment techniques, and providing substantial benefits for both clinical and consumer applications.

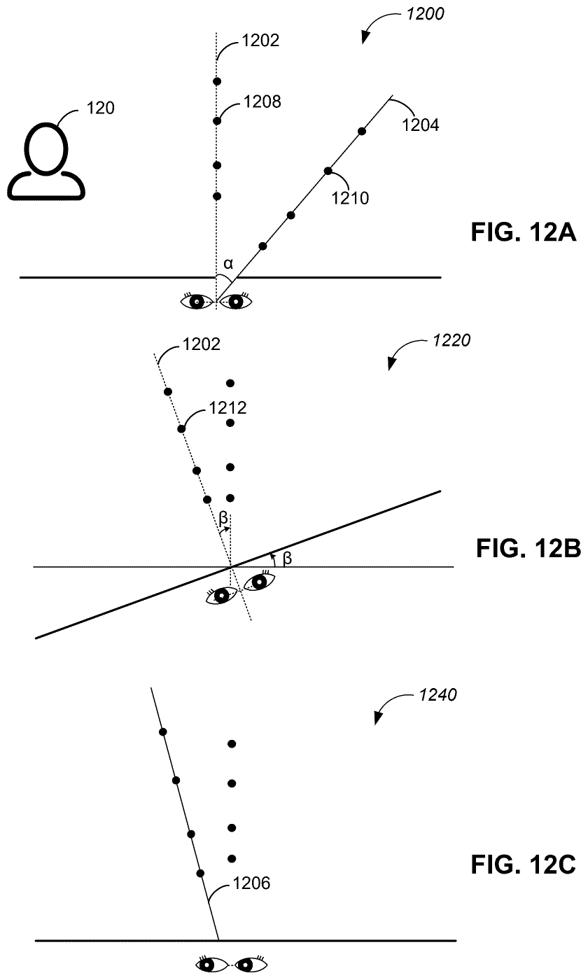

FIG. 12A is a diagram of an example field of view 1200 including two example lines of sight 1202 and 1204, in accordance with some embodiments. FIG. 12B is a diagram of an example field of view 1220 in which a line of sight 1202 changes with a head orientation, in accordance with some embodiments. FIG. 12C is a diagram of an example field of view 1240 in which a line of sight 1206 changes with eye positions, in accordance with some embodiments. A headset device 140D may execute a user application (e.g., a visual assessment application 328) configured to enable a virtual vision test and generate a VR user interface corresponding to a 3D virtual environment. A line of sight (e.g., lines 1202, 1204, and 1206) may correspond to a straight unobstructed path between a user 120 wearing the headset device 140D and a location in the 3D virtual environment, where the location is occupied by an object or corresponds to a remote point. In some embodiments, the visual assessment application 328 may display a visual stimulus at a location (e.g., location 1208 or 1210) on the line of sight.

Referring to FIG. 12A, in some embodiments, when the user 120 faces and looks forward, the line of sight 1202 may be perpendicular to a line connecting two eyeballs and presumed to have an angle of 0 degree. When the user 120 may rotate his eyes to look towards a right direction, the line of sight 1204 may not be perpendicular to the line connecting two eyeballs and can have a first angle α.

Referring to FIG. 12B, in some embodiments, the user 120 may rotate his head by a second angle β to result in the head orientation, which faces a direction shifting from a center line towards the left of the user 120 by the second angle β. The line of sight 1202 rotates with the user's head, and virtual content rendered in the field of view 1220 may not change with the head orientation. The 3D virtual environment does not rotate with the user's head. In some situations not shown, the user 120 may keep the head orientation and turn his eyes to the right by the second angle β to review the field of view 1200.

Referring to FIG. 12C, in some embodiments, the user 120 may not rotate his head by the second angle β, maintaining the head orientation facing forward. The user 120 may rotate his eyes towards left, thereby shifting a line of sight 1206 to the left by the second angle β. The virtual content reviewed by the user 120 in the field of view 1240 may be substantially consistent with that reviewed by the user 120 in the field of view 1220.

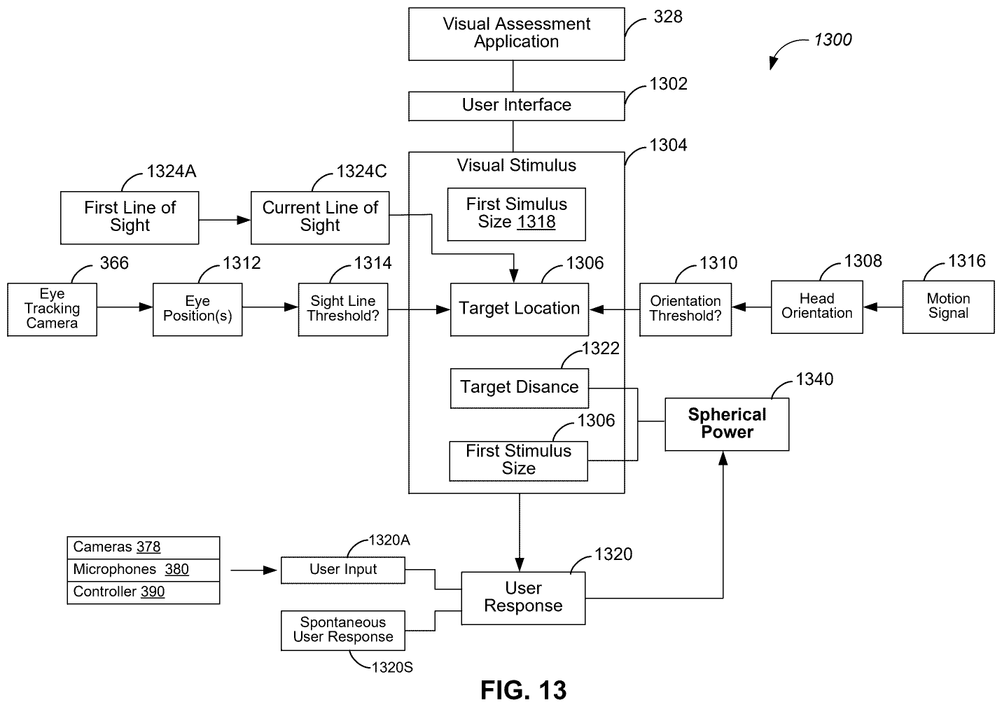

FIG. 13 is a flow diagram of an example vision test process 1300 for providing a visual stimulus 1304 adaptively based on a head orientation 1308, in accordance with some embodiments. A computer device 140 (e.g., headset device 140D) may include an HMD 312A, and one or more cameras 310A (e.g., outward camera 378 and eye-tracking camera 366 in FIG. 3). The computer device 140 may execute a user application (e.g., a visual assessment application 328) configured to enable a virtual vision test and generate a VR user interface 1302 corresponding to a 3D virtual environment. A visual stimulus 1304 corresponds to the virtual vision test, and may be displayed at a target location 1306 in the 3D virtual environment. In some embodiments, the visual stimulus 1304 includes a virtual object, e.g., text blocks, shapes, and interactive elements. The visual stimulus 1304 may be displayed at different distances from the user 120 within the 3D virtual environment. In some situations, the visual stimulus 1304 may be displayed with different lighting scenarios such as daylight, dusk, and night, as well as different contrasts and colors to challenge the user's visual acuity in diverse settings.

The computer device 140 may monitor a head orientation 1308 of a user 120 wearing the computer device 140, and dynamically adjust the target location 1306 of the visual stimulus 1304 based on the head orientation 1308. In some embodiments, the computer device 140 may obtain a motion signal 1316 measured by an integrated motion sensor 376 (FIG. 3), which may include an accelerometer and/or a gyroscope, and the head orientation 1308 may be determined based on the motion signal 1316.

In some embodiments, the computer device 140 may identify a standard line of sight 1202 (FIG. 12A) that extends forward from a center of, and is perpendicular to, a line connecting two eyes of the user 120. The target location 1306 (e.g., location 1208 in FIG. 12A) is selected on the standard line of sight 1202.

In some embodiments, the target location 1306 may correspond to a first orientation (e.g., corresponding to FIG. 12A). The computer device 140 may determine that the head orientation has stabilized at a current orientation (e.g., corresponding to FIG. 12B) distinct from the first orientation for a first extended duration of time. In accordance with a determination with the first extended duration of time is greater than an orientation threshold 1310 (e.g., 5 seconds), the target location 1306 may be moved to follow the current orientation. For example, referring to FIGS. 12A and 12B, after the user 120 rotates his head towards the left by the second angle β and stabilizes for more than 5 seconds, the target location 1306 where the visual stimulus 1304 is displayed may move from the location 1208 to the location 1212, following the head orientation.

In some embodiments, the computer device 140 may monitor an eye position 1312 based on eye images captured by an eye-tracking camera 366. The target location 1306 of the visual stimulus 1304 may be dynamically adjusted based on both the head orientation 1308 and the eye position 1312. Further, in some embodiments, the target location 1306 may correspond to a first line of sight 1324A (e.g., line of sight 1202 in FIG. 12A). The computer device 140 determines that the eye position 1312 has stabilized along a current line of sight 1324C (e.g., line of sight 1204 in FIG. 12A), where the current line of sight 1324C may be distinct from the first line of sight 1324A for a second extended duration of time. In accordance with a determination with the second extended duration of time is greater than a sight line threshold 1314 (e.g., 3 seconds), the target location 1306 may be moved to follow the current line of sight 1324C. For example, referring to FIGS. 12A and 12B, after the user 120 moves his eyes towards the right by the first angle α and stabilizes for more than 3 seconds, the target location 1306 where the visual stimulus 1304 is displayed may move from the location 1208 to the location 1210, following movement of the user's eyes.

In some embodiments, the visual stimulus 1304 has a first stimulus size 1318 at the target location 1306. While keeping the first stimulus size 1318, the computer device 140 may display the visual stimulus 1304 at one or more alternative locations that are different from the target location 1306, and receive a user response 1320 indicating that the visual stimulus 1304 starts to be clear at the target location 1306 to the user 120 compared with the one or more alternative locations. Alternatively, in some embodiments, the visual stimulus 1304 has a first stimulus size 1318 at the target location 1306. While displaying the visual stimulus 1304 at the target location 1306, the computer device 140 vary a size of the visual stimulus 1304 to one or more alternative stimulus sizes and receive a user response 1320 indicating that the visual stimulus 1304 starts to be clear at the first stimulus size 1318 to the user 120 compared with the one or more alternatively stimulus sizes.

In some embodiments, the computer device 140 may determine a target distance 1322 between the target location 1306 and the user 120. The computer device may determine a spherical power 1340 for vision correction for the user 120 based on the target distance 1322 and the first stimulus size 1318.

In some embodiments, the computer device 140 may receive a user response 1320 indicating whether the visual stimulus 1304 is clear to the user 120. The user response 1320 includes a user input 1320A captured by one or more first sensors of the computer device 140. The one or more first sensors include a forward-facing camera 378 (FIG. 3) for detecting a hand gesture, a microphone 380 (FIG. 3) for collecting an audio response, or a controller 390 (FIG. 3) for receiving a user physical force.

In some embodiments, the user response 1320 may include a spontaneous user response 1320S monitored by one or more second sensors of the computer device 140. The one or more second sensors include one or more of: an eye tracking camera 366, a heart rate sensor, a body temperature sensor, a blood oxygen level, a Galvanic skin response sensor, a hand gesture camera (e.g., camera 378), a body gesture camera (e.g., camera 378), a microphone 380, a motion sensor 376, and a set of one or more brain activity electrodes 362. In some embodiments, the eye tracking camera 366 may monitor gaze point, pupil size, and saccadic movements (quick, simultaneous movements of both eyes in the same direction). The spontaneous user response 1320S may be automatically determined based on image data captured by the eye tracking camera 366. More specifically, in some embodiments, the image data captured by the eye tracking camera 366 may be processed (e.g., by a machine learning model 350 in FIGS. 3 and 4) to determine a focal point of the user's eyes, a pupil size variation, a reaction time, and a consistency level across a plurality of vision tests.

In some embodiments, the computer device may select a first line of sight 1324A (e.g., line of sight 1202 or 1204 in FIG. 12A) including a plurality of locations (e.g., four locations marked on line of sight 1202 or 1204 in FIG. 12A). A visual stimulus 13004 has a fixed stimulus size, and may be displayed successively on the plurality of locations in the 3D virtual environment. A head orientation 1310 of a user 120 wearing the electronic device is monitored (e.g., by a motion sensor 376). The location of the visual stimulus is dynamically adjusted based on the head orientation 1310 to keep the visual stimulus 1304 on the first line of sight 1324A (e.g., line of sight 1202 or 1204 in FIG. 12A).

In an example, embodiments, the visual stimulus 1304 includes a text block, and may be displayed successively on the line of sight 1202 (FIG. 12A) at virtual distances of one meter, two meters, and five meters away from the user 120. In another example, the visual stimulus 1304 includes a virtual object having a 3D shapes (e.g., sphere, cube). The computer device 140 moves the virtual object successively among different depths (e.g., corresponding to different locations along the line of sight 1202 or 1204, prompting the user to refocus eyes continuously. In some embodiments, the visual stimulus 1304 includes a virtual object changing a respective distance from the user 120 with a fixed or varying speed, thereby assessing the user's dynamic focus ability and acuity level with moving objects.

### Cylinder Correction Assessment Using Dynamic Rotating Visual Fields

Some implementations of this application include a VR-based computer system 300 configured to assess cylinder correction (e.g., corresponding to astigmatism) by utilizing dynamic and rotating visual fields. The computer system 300 may utilize a high-resolution VR headset, which is equipped with precision eye-tracking sensors (e.g., eye-tracking cameras 366 in FIG. 3) and specialized software algorithms to generate dynamic visual stimuli that rotate in various patterns and speeds. Users may wear the VR headset and are exposed to a series of visual tests involving rotating lines, grids, and objects. The eye-tracking sensors may monitor the user's eye movements and visual responses to these dynamic stimuli, while the software algorithms analyze these responses to determine a degree and an axis of astigmatism, thereby determining cylinder correction needed by the user.

In some embodiments, the visual assessment application 328 implemented in the 3D virtual environment may include a variety of visual challenges, such as rotating lines that change orientation and speed, grids that warp dynamically, and objects that rotate around different axes. These tests are configured to challenge the user's visual perception and identify distortions caused by astigmatism. The application 328 may process eye-tracking data in real time, and measure the user's ability to focus on and track these rotating visual fields. Results may be compiled into a report that provides measurements of the cylinder correction required, along with the specific axis of astigmatism. The application 328 may offer a non-invasive, engaging, and accurate method for assessing and correcting astigmatism.

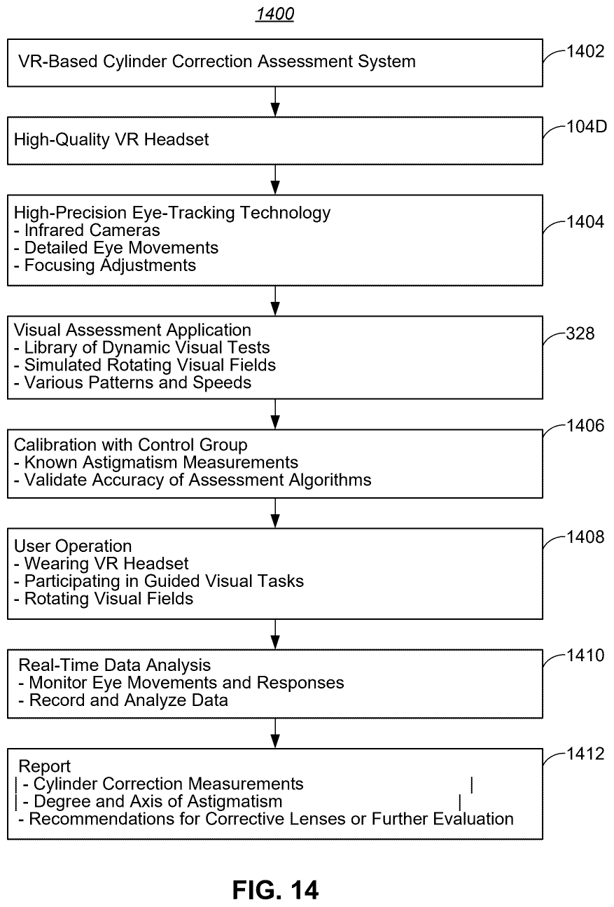

FIG. 14 is a flow diagram of an example vision test process 1400 for determining cylinder correction parameters of eyes of a user, in accordance with some embodiments. The VR-based computer system 300 is configured to enable a cylinder correction assessment system 1402. The computer system 300 may include a VR headset 104D that includes an eye-tracking camera 366 (FIG. 3). The eye-tracking technology 1404 may include an infrared camera (e.g., camera 366) configured to capture eye movements and focusing adjustments. In some embodiments, when a visual assessment application 328 is executed, a library of dynamic visual tests that simulate rotating visual fields in various patterns and speeds. The dynamic visual tests may be implemented to assess the user's ability to perceive and track dynamic fields accurately, thereby determining the degree and axis of astigmatism for the user's eyes.

In some embodiments, when hardware components and software modules may be integrated to form the spherical power measurement system, the VR-based computer system 300 may be calibrated (operation 1406) using a control group of individuals with known astigmatism measurements, thereby validating the accuracy of the assessment algorithms. Users can operate (operation 1408) the system by wearing the VR headset and participating in the guided visual tasks. The eye-tracking camera 366 may monitor eye movements and responses to the rotating visual fields. Image or video data recorded by the camera 366 may be analyzed (operation 1410) in real time by the software modules (e.g., visual assessment application 328, data processing module 330 in FIG. 3). In some implementations, the user may receive a report 1412 outlining cylinder correction measurements needed, including the degree and axis of astigmatism,, and the report may provide recommendations for corrective lenses or further ophthalmic evaluation. By these means, the computer system 300 may offer a precise, non-invasive, and user-friendly method for assessing and correcting astigmatism, representing a significant advancement over traditional refractive assessment techniques and providing substantial benefits for both clinical and consumer applications.

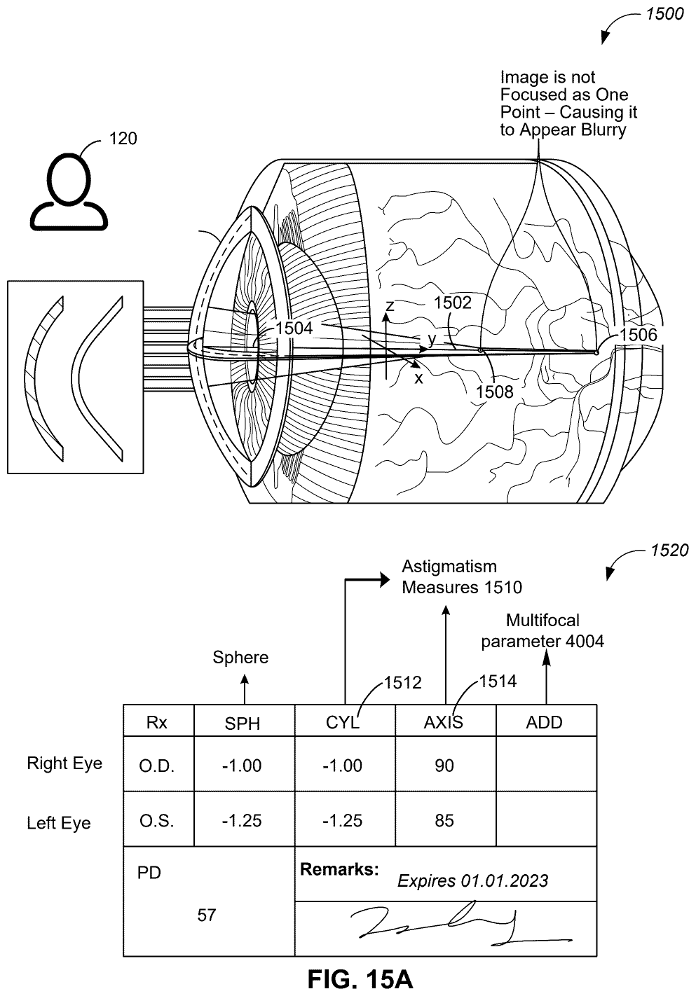

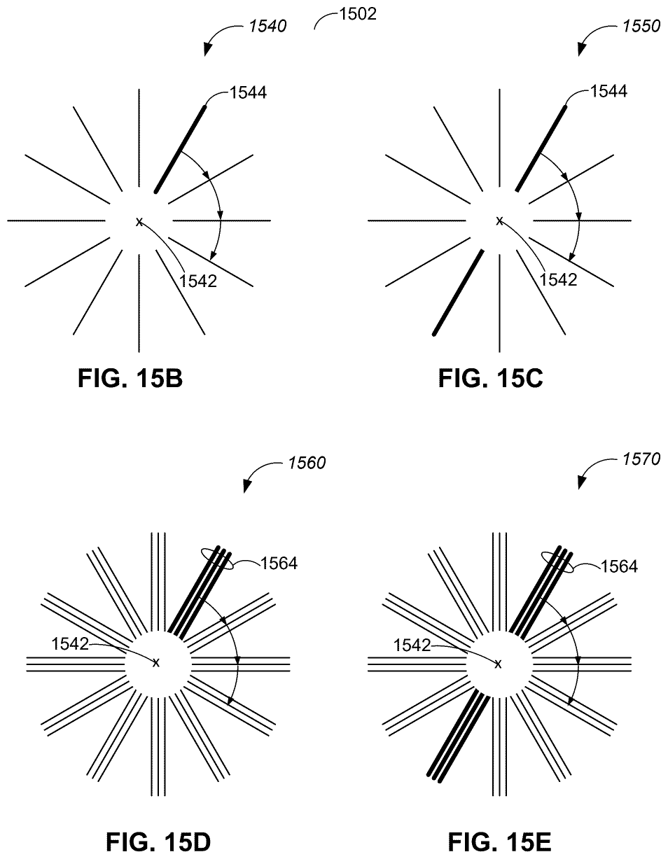

FIG. 15A is a cross sectional view of an example human eyeball 1500 and an associated prescription 1520, in accordance with some embodiments. The human eyeball 1500 includes a focal line 1502 connecting a center 1504 of a pupil and a focal point 1506 on a retina, and light entering the pupil from the center 1504 may propagate along the focal line 1502 until it hits the focal point 1506. A meridian surface is defined to include the center 1504 of the pupil and the focal line 1502, and light propagating on each meridian surface is focused at a respective focal point (e.g., point 1508) that may be in front of, on, or behind the retina. When the respective focal point does not land on the retina, the light propagating on the respective meridian surface may scatter on the retina. For example, the focal line 1502 extends along a y-axis of a coordinate system, and light propagating on a horizontal meridian surface defined by an x-axis and the y-axis may be focused at the focal point 1506 on the retina. Light propagating on a surface defined by a z-axis and the x-axis may be focused at the focal point 1508 in front of the retina and scattered when the light arrives at the retina. A cornea of the eyeball 1500 may not be regular, causing an astigmatism condition in which the light propagating on different meridian surfaces is focused at different focal points that may not overlap and could spread in front of, on, or behind the retina.

The astigmatism condition may be quantitatively assessed using astigmatism measures 1510 of each of the two eyes. For each eye, the astigmatism measures 1510 include a respective cylinder indicator 1512 (CYL) measuring a lens power for correcting astigmatism and a respective axis indicator 1514 measuring an orientation of astigmatism correction in degrees (e.g., 90 degrees, 85 degrees).

FIGS. 15B-15E are diagrams illustrating four astigmatism schemes 1540, 1550, 1560, and 1570 applied to assess an astigmatism condition in a 3D virtual environment, in accordance with some embodiments. An astigmatism wheel (e.g., pattern 630 in FIG. 6B) is a visual tool used in an eye exam to help diagnose astigmatism. The wheel includes a set of straight-line segments radiating outward from a central point 1542, arranged in a circular pattern. When a person with astigmatism looks at the wheel, some lines may appear darker or sharper than others, while other lines may appear blurred or faded. This distortion occurs because the cornea or lens of the eye is irregularly shaped, causing light to refract unevenly. By identifying which lines are affected, eye care professionals can determine the severity and axis of astigmatism, aiding in the prescription of corrective lenses. In some embodiments, a computer device 140 (e.g., headset device 140D) may include an HMD 312A, and one or more cameras 310A (e.g., outward camera 378 and eye-tracking camera 366 in FIG. 3). The computer device 140 may execute a user application (e.g., a visual assessment application 328) configured to enable a virtual vision test and generate a VR user interface 1302 corresponding to a 3D virtual environment. The computer device 140 may display straight-line segments of the astigmatism wheel located at different angular positions with respect to the central point 1542 successively (e.g., not concurrently). The straight-line segments of the astigmatism wheel may be displayed consecutively according to a clockwise or counterclockwise order.

Referring to FIG. 15B, in some embodiments, a plurality of visual stimuli 1544 may radiate outward from the central point 1542, arranged in a circular pattern, and each visual stimulus 1544 may include a single straight-line segment. Every two immediately adjacent stimuli 1544 may be separated by a predefined angle (e.g., 30 degrees). The straight-line segments may be displayed successively (e.g. one after the other) according to a clockwise or counterclockwise order. After 12 straight-line segments are displayed, the astigmatism scheme 1540 may start to repeat. Referring to FIG. 15C, in some embodiments, a plurality of visual stimuli 1544 may radiate outward from the central point 1542, arranged in a circular pattern, and each pair of visual stimuli 1544 may include two straight-line segments that are aligned and may extend to the central point 1542. Every two immediately adjacent stimuli 1544 may be separated by a predefined angle (e.g., 30 degrees). The straight-line segments may be displayed in pair successively (e.g. one after the other) according to a clockwise or counterclockwise order. After every six pairs of straight-line segments are displayed, the astigmatism scheme 1550 may start to repeat.

Referring to FIG. 15C, in some embodiments, a plurality of visual stimuli 1564 may radiate outward from the central point 1542, arranged in a circular pattern, and each visual stimulus 1564 may include a set of three parallel straight-line segments. Every two immediately adjacent stimuli 1564 may be separated by a predefined angle (e.g., 30 degrees). The straight-line segments may be displayed successively (e.g. one after the other) according to a clockwise or counterclockwise order. After 12 sets of straight-line segments are displayed, the astigmatism scheme 1560 may start to repeat. Referring to FIG. 15D, in some embodiments, a plurality of visual stimuli 1564 may radiate outward from the central point 1542, arranged in a circular pattern, and each pair of visual stimuli 1564 may include two sets of three straight-line segments that are symmetric with respect to the central point 1542 and may extend to the central point 1542. Every two immediately adjacent stimuli 1564 may be separated by a predefined angle (e.g., 30 degrees). The sets of straight-line segments may be displayed in pair successively (e.g. one after the other) according to a clockwise or counterclockwise order. After every six pairs of straight-line segment sets are displayed, the astigmatism scheme 1570 may start to repeat.

FIG. 16 is a flow diagram of an example vision test process 1600 for determining one or more astigmatism parameters 1620, in accordance with some embodiments. A computer device 140 (e.g., headset device 140D) may include an HMD 312A, and one or more cameras 310A (e.g., outward camera 378 and eye-tracking camera 366 in FIG. 3). The computer device 140 may execute a user application (e.g., a visual assessment application 328) configured to enable a virtual vision test and generate a VR user interface corresponding to a 3D virtual environment. A video clip 1602 may be displayed in the VR user interface corresponding to the 3D virtual environment. The video clip 1602 may include a plurality of image frames, and each image frame includes a predefined visual stimulus 1604 having a respective orientation 1606 with respect to a focal point (e.g., central point 1542 in FIGS. 15A-15D). While displaying the video clip 1602, the computer device 140 obtains eye image data 1608 (e.g., a sequence of eye images) of an eye of a user (e.g., a left eye, a right eye, or both), the computer device 140 may extract eye response data 1610 including a pupil size (P) 1612 from the eye image data 1608. A spontaneous user response 1614 to the video clip 1602 may be determined based on eye response data 1610, and applied to automatically determine one or more astigmatism parameters 1620.

In some embodiments, the eye image data may include a sequence of eye images 1618 of the eye, and each eye image 1618 corresponds to a respective pupil size value, and the pupil size (P) 1612 may vary with the plurality of eye images 1618. Further, in some embodiments, a pupil size extraction model 1616 may be applied to process each eye image 1618 and determine the respective pupil size value corresponding to the respective eye image 1618. Additionally, in some embodiments, the one or more astigmatism parameters 1620 may include an astigmatism axis 1620A of the eye, and a pupil astigmatism model 1622 may be applied to process the pupil size (P) 1612 that varies with the sequence of eye images 1618 and determine at least the astigmatism axis 1514. In some embodiments, the one or more astigmatism parameters 1620 may include a cylindrical power 1512 of the eye, and the pupil astigmatism model 1622 may be applied to process the pupil size 1612 that varies with the sequence of eye images 1618 and determine the cylindrical power 1512 in addition to the astigmatism axis 1514.

Stated another way, in some embodiments, the one or more astigmatism parameters 1620 may include at least one of a cylindrical power 1512 and an astigmatism axis 1514 of the eye. A pupil astigmatism model 1622 may be applied to process the pupil size and determine the one or more astigmatism parameters 1620. Further, in some embodiments, the computer device 140 may be communicatively coupled to a server 102. The server 102 may obtain an astigmatism parameter ground truth and a set of samples of a pupil size, and train the pupil astigmatism model 1622 based on the set of samples of the pupil size and the astigmatism parameter ground truth. The server 102 may provide the pupil astigmatism model 1622 to the computer device 140 (e.g., the headset device 140D).

In some embodiments, the predefined visual stimulus 1604 may include at least a straight-line segment 1544 (FIG. 15A) that is aligned with the focal point (e.g., central point 1542 in FIGS. 15A-15D). Alternatively, in some embodiments, the predefined visual stimulus 1604 includes two straight-line segments 1544 (FIG. 15B) that are aligned with the focal point, and the two straight-line segments 1544 are symmetric with each other with respect to the focal point. Alternatively, in some embodiments, the predefined visual stimulus 1604 includes at least a first set of two or more straight-line segments 1564 (FIG. 15C) that are closely disposed and parallel to each other, and each line segment is symmetric to a distinct line segment with respect to the focal point. Alternatively, in some embodiments, the predefined visual stimulus 1604 includes two identical sets of two or more straight-line segments 1564 (FIG. 15D), and the two sets of two or more straight-line segments of line segments are symmetric to each other with respect to the focal point.

In some embodiments, the predefined visual stimulus 1604 may be displayed at a distance and rotate continuously (operation 1624) with respect to the focal point in the video clip 1602 for a plurality of cycles (e.g., 5 cycles). Further, in some embodiments, the predefined visual stimulus 1604 may have a rotation speed 1626 that is below a threshold speed, and the plurality of cycles include a number of cycles 1628 that is within a range of cycle numbers. For example, the threshold speed is 5 cycles per minute, and the range of cycle numbers is 2-10 inclusively.

In some embodiments, the predefined visual stimulus 1604 may be displayed at a distance and rotate with respect to the focal point in the video clip 1602 based on a plurality of discrete angular positions 1630 (e.g., every 30° within a range of 0-360°).

Some implementations of this application are directed to vision testing in a 3D virtual environment. A computer device 140 may display a video clip 1602 including a plurality of image frames, and each image frame includes a predefined visual stimulus 1604 having a respective orientation 1606 with respect to a focal point (e.g., central point 1542 in FIGS. 15A-15D), such that the predefined visual stimulus 1604 is displayed rotating continuously with respect to the focal point in the video clip 1602. While displaying the video clip 1602, the computer device 140 may obtain eye image data 1608 of an eye of a user 120, and determine a user response 1614 to the video clip 1602 based on the eye image data 1608. One or more astigmatism parameters 1620 may be automatically determined based on the user response 1614. In some embodiments, the user response 1614 includes a pupil size 1612, and the computer device may apply a pupil size extraction model 1616 to process each eye image 1618 and determine a respective pupil size value corresponding to the respective eye image 1618. Further, in some embodiments, the one or more astigmatism parameters 1620 include an astigmatism axis 1514 of the eye. A pupil astigmatism model 1622 may be applied to process the pupil size 1612 that varies with a plurality of eye images 1618 of the eye image data and determine at least the astigmatism axis 1514. Additionally, the one or more astigmatism parameters 1620 may further include a cylindrical power 1512 of the eye, and the pupil astigmatism model 1622 may be applied to process the pupil size 1612 and determine the cylindrical power 1512 in addition to the astigmatism axis 1514.

### Three-dimensional (3D) Depth Perception in a Virtual Environment

Some implementations of this application include a VR-based computer system 300 configured to test depth perception using 3D objects and immersive virtual environments. This innovative system comprises a high-resolution VR headset equipped with precision eye-tracking technology and advanced software capable of rendering three-dimensional visual stimuli. Users wear the VR headset and engage in a series of interactive tasks that involve manipulating and interacting with 3D objects within a virtual space. These tasks may be configured to assess various aspects of depth perception, including stereopsis (binocular vision), spatial awareness, and distance judgment. The eye-tracking sensors may monitor the user's eye movements and focusing behaviors, while the software analyzes these responses to evaluate the user's depth perception capabilities.

In some embodiments, the VR-based computer system 300 may identify relative distances between objects, navigate through complex 3D environments, and perform tasks that require precise depth judgments, e.g., catching virtual objects or threading through spatial mazes. The VR-based computer system 300 may collect data indicating how accurately and efficiently the user completes these tasks, and process the data with advanced algorithms (e.g., machine learning models 350 in FIG. 3) to assess depth perception accuracy and identify potential deficiencies. Results may be compiled into a detailed report that indicates the user's depth perception performance and provides insights for diagnosing conditions (e.g., strabismus, amblyopia, or convergence insufficiency). As such, in some embodiments, the computer system 300 may provide a dynamic, engaging, and accurate method for evaluating depth perception in a controlled virtual environment.

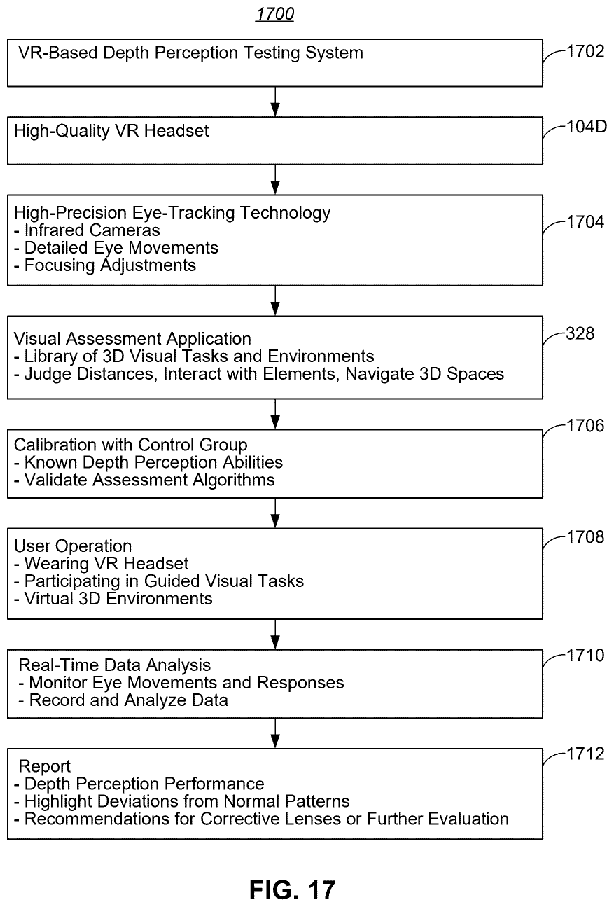

FIG. 17 is a flow diagram of an example vision test process 1700 for assessing a depth perception level of a user's eyes, in accordance with some embodiments. The VR-based computer system 300 is configured to enable the VR-based depth perception testing system 1702. The computer system 300 may include a VR headset device 104D that includes an eye-tracking camera 366 (FIG. 3). The eye-tracking technology 1704 may include an infrared camera (e.g., camera 366) configured to capture eye movements and focusing adjustments. In some embodiments, when a visual assessment application 328 is executed, a library of visual tasks is applied to test different aspects of depth perception. These tasks may include scenarios where users may judge distances between objects, interact with virtual elements, and navigate through 3D spaces, all configured to challenge and measure depth perception.

In some embodiments, when hardware components and software modules may be integrated to form the depth perception testing system, the VR-based computer system 300 may be calibrated (operation 1706) using a control group of individuals with known depth perception abilities to validate the accuracy of the assessment algorithms. Users can operate (operation 1708) the calibrated computer system 300 by wearing the VR headset and participating in the guided visual tasks. The eye-tracking camera 366 may monitor focus adjustments and visual responses of a user's eyes to 3D stimuli. Image or video data recorded by the camera 366 may be analyzed (operation 1710) in real time by the software modules (e.g., visual assessment application 328, data processing module 330 in FIG. 3). In some implementations, the user may receive a report 1712 outlining eye depth perception performance, and the report may indicate deviations from normal patterns and provide recommendations for corrective measures or further medical consultation. By these means, the computer system 300 may offer a precise, non-invasive, and user-friendly method for assessing depth perception, representing a significant advancement on depth perception testing techniques and providing substantial benefits for both clinical and consumer applications.

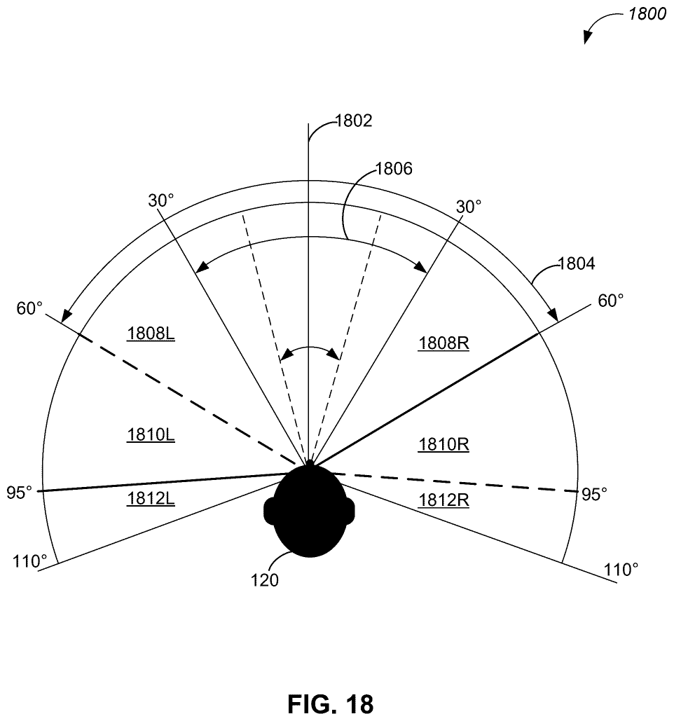

FIG. 18 is a diagram of an example horizontal field of view (HFOV) 1800 of a user's eyes, in accordance with some embodiments. The HFOV 1800 refers to the extent of a visual field that the user 120 can see from side to side, measured in degrees. The HFOV 1800 may include a monocular view of each eye of the user 120 referring to a portion of the HFOV 1800 perceived by the respective eye at a time. In some embodiments, for a single eye, the HFOV 1800 is typically around 155 degrees, depending on the user's eye anatomy. For example, a reference axis 1802 extends forward from a middle point of a line connecting the user's two eyes. A left monocular view of a left eye covers an angular range from −95° to +60° with respect to the reference axis 1802,and a right monocular view of a right eye covers an angular range from −60° to +95° with respect to the reference axis 1802. If only one eye is used, depth perception may be limited, and an object may appear flatter compared to when use both eyes.

The left monocular view of the left eye and the right monocular view of the right eye may overlap in a binocular area 1804 covering a binocular angular range (e.g., [−60°, 60°]). The binocular area 1804 occurs when both eyes work together, allowing for depth perception and a more accurate representation of 3D space. The binocular area 1804 is where stereoscopic vision occurs, providing depth and spatial awareness. Stereoscopic vision is the ability to perceive depth and three-dimensional structure by integrating visual information from both eyes. Each eye captures a slightly different image because they are spaced apart (about 6-7 cm in humans), giving each eye a unique angle on the same object. The user's brain processes and merges these two images associated with two eyes to create a single 3D perception, which is a process known as binocular fusion.

The binocular area 1804 may include an area of focus 1806 (e.g., from −30° to 30°), a left peripheral area 1808L, and a right peripheral area 1808R. For example, the peripheral area 1808L or 1808R is about 30 degrees. The HFOV 1800 further includes a left edge area 1810L that is only visible to the left eye and a right edge area 1810R that is only visible to the right eye. The left edge area 1810L and the right edge area 1810R are immediately adjacent to the binocular area 1804. Each of the edge areas 1810L and 1810R may cover an angular range of 35°. Additionally, the HFOV 1800 is further expanded by a temporal area 1812L or 1812R (e.g., corresponding to 15°) on each of two sides of the user's head. The binocular area 1804, the edge areas 1810L and 1810R, and the temporal area 1812L and 1812R contribute to the overall perception of the surrounding environment, with the binocular area 1804 providing enhanced depth and spatial information crucial for activities like driving, sports, or reading depth cues in daily life. In some situations, the user 120 has an impaired HFOV in which the binocular area 1804 covers less than a normal binocular angular range (e.g., the impaired HFOV spans from −40° to 60°, rather than from −60° to 60°). The user's depth perception is compromised within part of the normal binocular angular range.

FIG. 19 is a flow diagram of an example vision test process 1900 for determining a depth perception level 1920 of a user, in accordance with some embodiments. A computer device 140 (e.g., headset device 140D) may include an HMD 312A, and one or more cameras 310A (e.g., outward camera 378 and eye-tracking camera 366 in FIG. 3). The computer device 140 may execute a user application (e.g., a visual assessment application 328) configured to enable a virtual vision test and generate a VR user interface corresponding to a 3D virtual environment. A first visual stimulus 1902 may be displayed at a first depth D1 in the user interface, and the first depth D1 may be measured on a first line of sight 1904. The first visual stimulus may be displayed at a second depth D2 in the user interface. The second depth D2 is distinct from the first depth D1 and measured on the first line of sight 1904. The computer device 140D obtains one or more user responses 1906 to displaying of the first visual stimulus 1902, e.g., at the depths D1 and D2. Based on the one or more user responses 1906, the computer device 140D determines the depth perception level 1920 for a user 120 associated with the computer device 140D.

In some embodiments, in accordance with a determination that the one or more user responses 1906 indicate that the user recognizes that the first depth D1 is different from the second depth D2, the computer device 140D may determine that the depth perception level 1920 (e.g. a depth resolution) is at least a difference of the first depth D1 and the second depth D2.

In some embodiments, the first stimulus 1902 may be displayed on the first depth D1 and the second depth D2 concurrently. Alternatively, in some embodiments, the first stimulus 1902 may be displayed on the first depth D1 and the second depth D2 sequentially. In some embodiments, a size of the first stimulus 1902 is displayed adaptively with the first depth D1 and the second depth D2.

In some embodiments, the computer device 140D may present a prompt 1908 requesting the user 120 to indicate whether the user 120 can visually differentiate the first depth D1 from the second depth D2. Stated another way, the prompt 1908 requests the user 120 to confirm whether the user 120 can recognize a depth resolution DR1 (e.g., equal to D2−D1) at a depth D1, D2, or an average of D1 and D2.

In some embodiments, the HMD may include a left display 1910L associated with a left eye of the user 120 and a right display 1910R associated with a right eye of the user 120. The computer device 140 may concurrently display the first visual stimulus 1902 at a left position in the left display 1910L and at a right position in the right display 1910R. The left position is distinct from the right position. Stated another way, in some embodiments, the computer device 140D may render a first version of the first visual stimulus 1902 on a left display 1910L, and a second version of the first visual stimulus 1902 on a right display 1910R. The first version and the second version may be different from one another, thereby creating the first depth in the user's eyes.

In some embodiments, the computer device 140D may identify a target depth 1912 associated with the depth perception level 1920. The target depth 1912 may be measured on the first line of sight 1904. The first depth D1 and the second depth D2 may be determined based on the target depth 1912. The first depth D1 and the second depth D2 may a first depth resolution DR1. Further, in some embodiments, for each of a plurality of depth resolutions DR2, the computer device 140D may determine a respective pair of depths based on the respective depth resolution DR2 and the target depth 1912. A respective visual stimulus (e.g., stimulus 1902) may be displayed at the respective pair of depths corresponding to the respective depth resolution DR2. The computer device 140D may obtain a respective user response. The depth perception level 1920 is determined for the user based on both the one or more user responses 1906 and the respective user responses corresponding to the plurality of depth resolutions DR2.

In some embodiments, the computer device 140D may scan a plurality of line depths including the target depth 1912 along the first line of sight 1904 to determine a respective depth perception level 1920 for each line depth on the first line of sight 1904.

In some embodiments, the computer device 140D may scan a plurality of line depths on each of a plurality of lines of sight 1904 and 1914 to determine a set of depth perception levels 1920 for the plurality of line depths on each line of sight 1904 or 1914. The plurality of line depths may be scanned on the first line of sight 1904 and include the target depth 1912 on the first line of sight 1904. Additionally, in some embodiments, the set of depth perception levels 1920 may be consolidated for the plurality of line depths on the plurality of lines of sight 1904 and 1914, forming a depth perception map 1916 for the user 120 based on depth perception level sets that are determined for the plurality of line depths on the plurality of lines of sight 1904 and 1914. For example, the depth perception map 1916 may correspond to a plurality of locations 1918 in a binocular area 1804 or in a focus area 1806, and include a line depth value and a depth resolution value (e.g., DR1) for each location 1918. When the depth resolution value is larger than a depth resolution threshold, the computer device 140 may determine that the user's depth perception level is impaired at a corresponding location 1918. Locations 1918 having impaired depth perception levels may be marked on the depth perception map 1916, indicating a severity level of the user's condition associated with a depth perception loss.

In some embodiments, the one or more user responses 1906 include a user input 1906A captured by one or more first sensors of the computer device 140. The one or more first sensors include a forward-facing camera 378 (FIG. 3) for detecting a hand gesture, a microphone 380 (FIG. 3) for collecting an audio response, or a controller 390 (FIG. 3) for receiving a user physical force.

In some embodiments, the user response 1906 may include a spontaneous user response 1906S monitored by one or more second sensors of the computer device 140. The one or more second sensors include one or more of: an eye tracking camera 366, a heart rate sensor, a body temperature sensor, a blood oxygen level, a Galvanic skin response sensor, a hand gesture camera (e.g., camera 378), a body gesture camera (e.g., camera 378), a microphone 380, a motion sensor 376, and a set of one or more brain activity electrodes 362. Alternatively or additionally, in some embodiments, the eye tracking camera 366 may monitor gaze point, pupil size, and saccadic movements (quick, simultaneous movements of both eyes in the same direction). The spontaneous user response 1906S may be automatically determined based on image data captured by the eye tracking camera 366. More specifically, in some embodiments, the image data captured by the eye tracking camera 366 may be processed (e.g., by an eye image analysis model) to determine a focal point of the user's eyes, a pupil size variation, a reaction time, and a consistency level across a plurality of visual stimuli.

In some embodiments, the computer device 140D may obtain a plurality of eye images 1922 of the user's eyes while the first stimulus 1902 is displayed at the first depth D1 and the second depth D2. Each eye image 1922 may corresponds to a respective eye focal length. Further, in some embodiments, a focus extraction model 1924 may be applied to process the plurality of eye images 1922 and determine two distinct eye focal lengths 1926 corresponding to the first depth D1 and the second depth D2. Automatically and without user intervention, the computer device 140 may determine whether the eye differentiates the first depth D1 from the second depth D2 based on the two distinct eye focal lengths 1926.

Some implementations of this application are directed to mapping depth perception levels (e.g., depth resolutions) of a user 120 within a HFOV 1800. A computer device 140 includes an HMD. The computer device 140 identifies a plurality of lines of sight 1904 and 1914. For each of the plurality of lines of sight 1904 or 1914, two visual stimuli may be displayed at two respective depths D1 and D2 surrounding each of a plurality of target depth 1912. A user response 1906 may be obtained, after the two stimuli are displayed at the two depths D1 and D2 surrounding each respective target depth 1912. Based on user responses associated with the respective target depths of the plurality of lines of sight, the computer device 140 may form a depth perception map 1916 for the user 120 associated with the electronic device. In some embodiments, the two visual stimuli may be displayed at the two respective depths D1 and D2 surrounding each respective target depth 1912 concurrently. Alternatively, in some embodiments, the two visual stimuli may be displayed at the two respective depths D1 and D2 surrounding each respective target depth 1912 sequentially. In some embodiments, for each of the plurality of target depths 1912, a difference of the two depths D1 and D2 (e.g., where the two visual stimuli are displayed) are gradually decreased until the corresponding user response 1906 indicates that the user 120 cannot recognize the difference of the two depths D1 and D2.

### Stereopsis Tests for Determining Depth Perception Profiles

Some implementations of this application include a VR-based computer system 300 configured to evaluate binocular vision through stereopsis tests using 3D images. The computer system 300 may utilize a high-resolution VR headset equipped with precision eye-tracking sensors (e.g., eye-tracking cameras 366 in FIG. 3) and specialized software algorithms to generate stereoscopic visual stimuli. Users wear the VR headset and engage in a series of stereopsis tests that present 3D images requiring the integration of visual information from both eyes to perceive depth and spatial relationships accurately. The eye-tracking sensors may monitor the user's eye alignment, convergence, and coordination, while the software analyzes these responses to provide a comprehensive assessment of binocular vision and depth perception.

In some embodiments, the VR-based computer system 300 may incorporate a range of visual tasks to evaluate different aspects of stereopsis. Examples of the visual tasks include, but are not limited to, identifying which object is closer, matching objects at different depths, and interacting with 3D virtual objects. These tests are conducted in immersive virtual environments that simulate real-world scenarios, challenging the user's binocular vision in various contexts. The software processes the data in real time, using advanced algorithms to assess the user's ability to perceive depth and coordinate eye movements effectively. The results are compiled into a detailed report that highlights any deficiencies in binocular vision, offering valuable insights for diagnosing conditions such as strabismus, amblyopia, or convergence insufficiency.

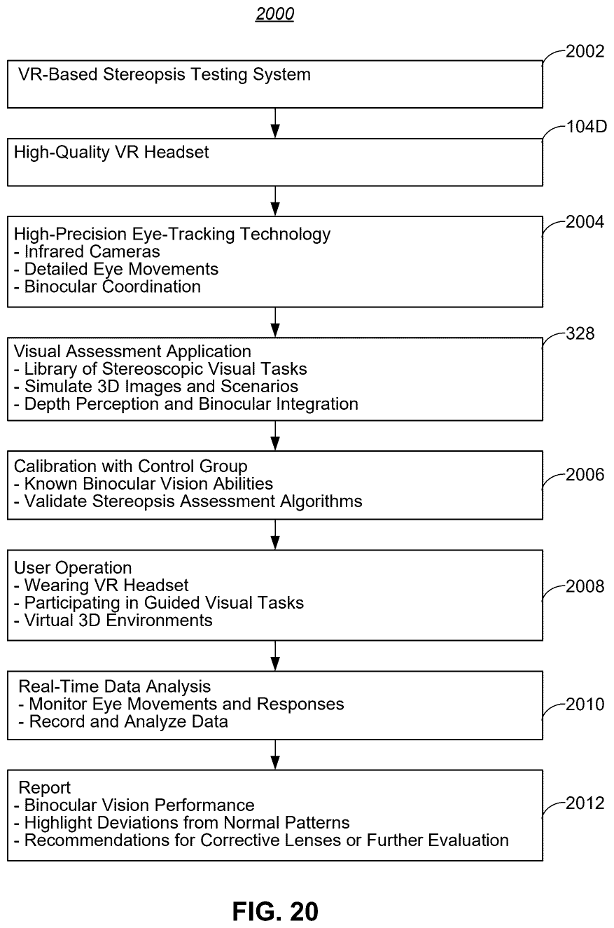

FIG. 20 is a flow diagram of an example vision test process 2000 for assessing a stereopsis condition of a user's eyes, in accordance with some embodiments. The VR-based computer system 300 is configured to enable a VR-based stereopsis testing system 2002. The computer system 300 may include a VR headset 104D that includes an eye-tracking camera 366 (FIG. 3). The eye-tracking technology 2004 may include an infrared camera (e.g., camera 366) configured to capture eye movements and binocular coordination. In some embodiments, when a visual assessment application 328 is executed, a library of stereoscopic visual tasks is applied to simulate 3D images and scenarios requiring depth perception and binocular integration. These tasks include activities where users may be prompted to differentiate distances between objects, align visual targets, and interact with virtual elements in a 3D virtual environment.

In some embodiments, when hardware components and software modules may be integrated to form the VR-based stereopsis testing system, the VR-based computer system 300 may be calibrated (operation 2006) using a control group of individuals with binocular vision abilities, thereby establishing baseline performance metrics and validate the accuracy of the stereopsis assessment algorithms. Users can operate (operation 2008) the system by wearing the VR headset and participating in the guided stereopsis tests within the virtual environments. Image or video data recorded by the camera 366 may be analyzed (operation 2010) in real time by the software modules (e.g., visual assessment application 328, data processing module 330 in FIG. 3). The eye-tracking sensors may monitor their eye movements and responses to the 3D stimuli, while the software records and analyzes the data in real time. The user receives a report 2012 outlining their binocular vision performance, highlighting any deviations from normal patterns, and providing recommendations for corrective measures or further medical consultation if necessary. This novel approach offers a precise, non-invasive, and user-friendly method for assessing stereopsis and binocular vision, representing a significant advancement over traditional testing techniques and providing substantial benefits for both clinical and consumer applications.

Stereopsis is the process by which the brain combines the two slightly different images from each eye into a single, 3D perception of depth. This ability arises because the eyes are spaced apart, giving each eye a slightly different view of the same scene. The brain uses the differences between these two images—called binocular disparity—to calculate depth and distance, allowing us to perceive the relative position of objects in space. Stereopsis is the foundation of depth perception in stereoscopic vision and is crucial for tasks that require precise spatial judgments, such as grasping objects, judging distances, or navigating through complex environments. Stereopsis depends on the proper alignment and coordination of both eyes. If the eyes do not work together (as in conditions like strabismus), stereopsis may be impaired, affecting depth perception. Some implementations of this application are directed to testing the user's stereopsis capabilities in a 3D virtual environment enabled by a headset device 140D.

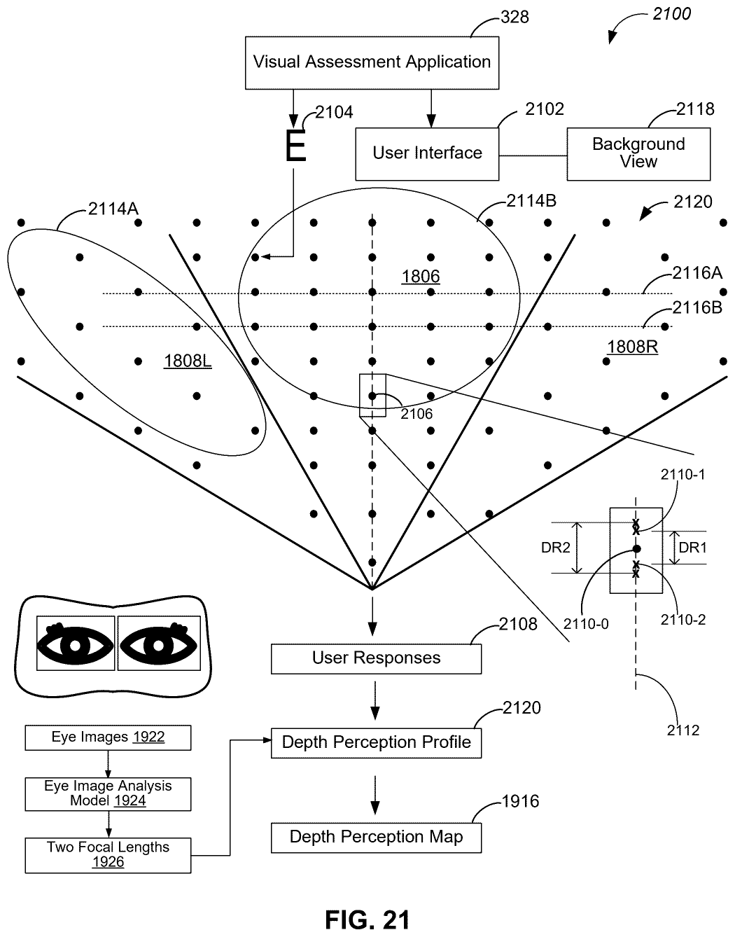

FIG. 21 is a flow diagram of an example vision test process 2100 for determining a depth perception profile 2120 for a user's eyes, in accordance with some embodiments. A computer device 140 (e.g., headset device 140D) may include an HMD 312A, and one or more cameras 310A (e.g., outward camera 378 and eye-tracking camera 366 in FIG. 3). The computer device 140 may execute a user application (e.g., a visual assessment application 328) configured to enable a virtual vision test and generate a VR user interface 2102 corresponding to a 3D virtual environment. The computer device 140 may display a plurality of visual stimuli 2104 (e.g., an optotype “E”) in the user interface 2102, and each visual stimulus 2104 may be displayed in duplication with respect to a respective target depth 2106, e.g., which is represented by a solid dot in FIG. 21. The computer device 140 may receive one or more user responses 2108. Each user response 2108 may indicate whether a user 120 perceives a corresponding visual stimulus 2104 in duplication at the respective target depth 2106. Based on the one or more user response 2108, the computer device 140 may determine a depth perception profile 2120 of the user 120. An example of the depth perception profile 2120 is a depth perception map 1916 (FIG. 19). The depth perception profile 2120 may include a plurality of depth perception levels 1920 corresponding to a plurality of target depths 2106.

In some embodiments, each visual stimulus 2104 may be displayed in duplication at a first position 2110-1 and a second position 2110-2. The first position 2010-1, the second position 2110-2, and an intermediate position 2110-0 between the first position 2110-1 and the second position 2110-2 may be aligned to one another on a respective line of sight 2112. The intermediate position 2110-0 may correspond to the respective target depth 2106.

In some embodiments, locations of the plurality of target depths 2106 where the visual stimulus 2104 is displayed in duplication are evenly distributed on the depth perception profile 2120. In some embodiments, locations of the plurality of target depths 2106 where the visual stimulus 2104 is displayed in duplication are not evenly distributed on the depth perception profile 2120. A first density of target depths 2106 may be applied to the area of focus 1806 (FIG. 18), and a second density of target depths 2106 may be applied to the left peripheral area 1808L and the right peripheral area 1808R. The first density may be greater than the second density. Stated another way, in some embodiments, the plurality of visual stimuli 2104 may be distributed in a binocular area 1804 (FIG. 18) of a field of view 1800 of a user 120 associated with the computer device 140. Further, in some embodiments, the binocular area 1804 includes a focus area 1806 and a peripheral area 1808L or 1808R. A first set of visual stimuli may be distributed in the focus area 1806 with the first density, and a second set of visual stimuli may be distributed in the peripheral area 1808L or 1808R with the second density. The first density may be greater than the second density.

In some embodiments, the plurality of visual stimuli 2104 may be displayed concurrently at the plurality of target depths 2106 on the user interface 2102.

In some embodiments, the plurality of visual stimuli 2104 may be divided into a plurality of groups of visual stimuli (e.g., a first group 2114A, a second group 2114B). Each group 2114A or 2114B of visual stimuli may be displayed concurrently on the user interface, and the plurality of groups of visual stimuli may be displayed successively on the user interface 2102. For example, the first group 2114A of visual stimuli may be displayed concurrently, and the second group 2114B of visual stimuli may be displayed concurrently, after the first group 2114A of visual stimuli are displayed.

In some embodiments, the one or more user responses 2108 include a user input captured by one or more first sensors of the computer device 140. Alternatively or additionally, in some embodiments, the user response 2108 may include a spontaneous user response monitored by one or more second sensors of the computer device 140. More details on the one or more user responses 2108 are discussed above with reference to the user response 1906 in FIG. 19. In some embodiments, the user 120 may be prompted to use a controller 390 (FIG. 3) to identify the visual stimulus 2104 that can or cannot be identified as being in duplication.

In some embodiments, the computer device 140 may obtain a plurality of eye images 1922 of the user's eyes while a first visual stimulus 2104 is displayed at a first depth (e.g., corresponding to the first position 2110-1) and a second depth (e.g., corresponding to the second position 2110-2) concurrently. Each eye image 1922 may correspond to a respective eye focal length. Further, in some embodiments, a focus extraction model 1924 may be applied to process the plurality of eye images 1922 and determine two distinct eye focal lengths 1926 corresponding to the first depth and the second depth. Automatically and without user intervention, the computer device 140 may determine whether the eye differentiates the first depth from the second depth based on the two distinct eye focal lengths 1926. The first depth and the second depth may correspond to a depth resolution DR1. When the user response 2108 indicates that the user 120 cannot differentiate the first depth and the second depth, the computer device may reduce the depth resolution to DR2 to test whether the user may differentiate the visual stimulus 2104 displayed in duplication.

In some embodiments, a set of visual stimuli 2104 displayed on a line of sight (e.g., line of sight 2112) may be displayed concurrently on respective heights without blocking each other. The plurality of visual stimuli 2104 may be displayed on the same height with respect to the eyes of the user. Different rows (e.g., 2116A and 2116B) of visual stimuli 2104 may be displayed successively without blocking each other.

In some embodiments, the plurality of visual stimuli 2104 are displayed on a background view 2118 to test the user's depth perception levels 1920 under different conditions (e.g., exposed to different lightings or disturbances). The computer device 140 may render a static image or a stream of video data associated with the background view 2118 on the user interface 2102. The plurality of visual stimuli 2104 may be overlaid on the static image or a set of respective image frames in the stream of video data associated with the background view 2118. Further, in some embodiments, the background view 2118 is one of: a static beach view, a static city night scene, and a dynamic traffic view. In some embodiments, the background view 2118 may include a brightness level and a contrast level. In some embodiments, the background view 2118 may include a doctor's office where the vision test is implemented. Stated another way, the vision test may be implemented in a 3D augmented reality environment.

### Eye Contrast Sensitivity in 3D Virtual Environments

Some implementations of this application include a VR-based computer system 300 configured to test contrast sensitivity of eyes using gradient patterns. The computer system 300 may utilize a high-resolution VR headset equipped with precision eye-tracking sensors (e.g., eye-tracking cameras 366 in FIG. 3) and specialized software algorithms to generate gradient visual stimuli. Users wear the VR headset and engage in a series of visual tasks that present gradient patterns with varying levels of contrast. The eye-tracking sensors may monitor the user's eye movements and fixation points, while the software analyzes these responses to assess the user's contrast sensitivity across different spatial frequencies. The VR-based computer system 300 may provide a detailed and dynamic method for evaluating contrast sensitivity, offering significant improvements over traditional static chart-based tests.

In some embodiments, the VR-based computer system 300 may display a variety of gradient patterns, such as sinusoidal gratings and concentric circles, displayed at different contrast levels and spatial frequencies. Users may identify and respond to the gradient patterns, and the gradient patterns may be dynamically adjusted based on the user's performance. A user application 324 (e.g., visual assessment application 328 in FIG. 3) may process the data in real time to determine contrast variations and identify patterns at different levels of difficulty. Results may be compiled into a report that describes the user's contrast sensitivity function and identifies deficiencies that could indicate underlying ocular or neurological conditions (e.g., glaucoma, cataracts, or optic neuritis).

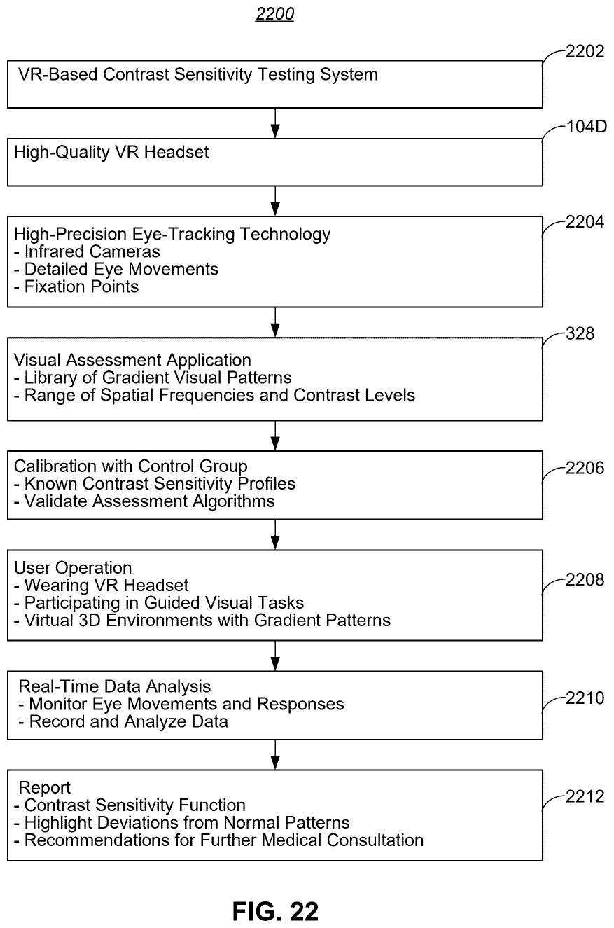

FIG. 22 is a flow diagram of an example vision test process 2200 for assessing a contrast sensitivity level of a user's eyes, in accordance with some embodiments. The VR-based computer system 300 may be configured to enable a VR-based contrast sensitivity testing system 2202. The computer system 300 may include a VR headset 104D that includes an eye-tracking camera 366 (FIG. 3). The eye-tracking technology 2204 may include an infrared camera (e.g., camera 366) configured to capture eye movements and fixation points. In some embodiments, when a visual assessment application 328 is executed, a library of gradient visual patterns may be applied to test different aspects of contrast sensitivity. The gradient visual patterns may include a range of spatial frequencies and contrast levels to challenge the user's visual system and measure contrast detection capabilities.

In some embodiments, when hardware components and software modules may be integrated to form the VR-based contrast sensitivity testing system, the VR-based computer system 300 may be calibrated (operation 2206) using a control group of individuals with known contrast sensitivity profiles, thereby establishing baseline performance metrics and validate the accuracy of the assessment algorithms. Users can operate (operation 2208) the system by wearing the VR headset and participating in the guided visual tasks within the virtual environments. The eye-tracking camera 366 may monitor eye movements and responses to the gradient patterns. Image or video data recorded by the camera 366 may be analyzed (operation 2210) in real time by the software modules (e.g., visual assessment application 328, data processing module 330 in FIG. 3). In some implementations, the user may receive a report 2212 outlining contrast sensitivity function, and the report may indicate deviations from normal patterns and provide recommendations for further medical consultation. By these means, the computer system 300 may offer a precise, non-invasive, and user-friendly method for assessing contrast sensitivity, representing a significant advancement over traditional refractive assessment techniques, and providing substantial benefits for both clinical and consumer applications.

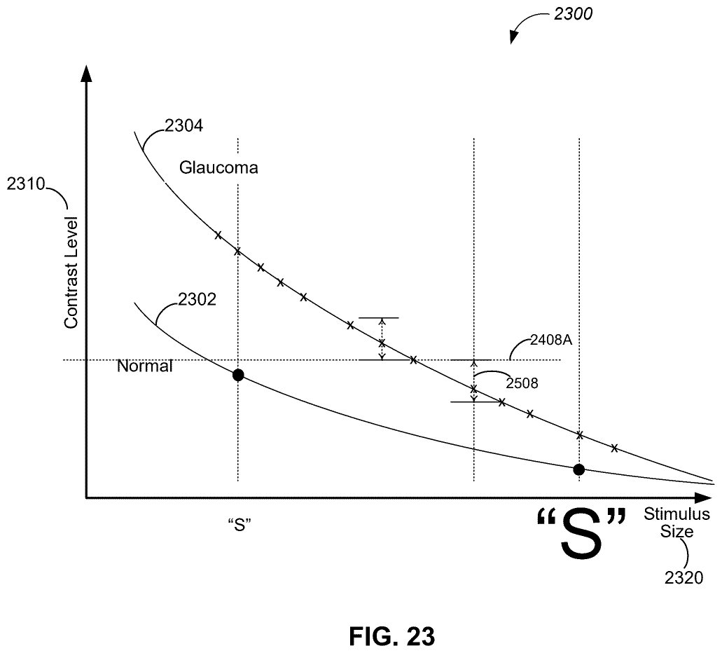

FIG. 23 is a plot 2300 of two example curves 2302 and 2304 representing correlations between a size of an object 2320 and a contrast level 2310 required for a person's eyes to recognize the object, in accordance with some embodiments. The size of the object 2320 corresponds to an acuity level. The curve 2302 may correspond to the correlation of a healthy eye, and the curve 2304 corresponds to the correlation of an eye having a Glaucoma condition. As the size of an object increases, the amount of contrast needed for recognition may decrease. Larger objects may offer more visual cues and are easier for the brain to detect, even in low-contrast conditions, while smaller objects need higher contrast to be easily distinguishable. This correlation is due to how the visual system processes spatial information: larger objects stimulate more retinal cells, making them more noticeable even when they blend into the background. Conversely, small objects with low contrast may go unnoticed because they provide less visual data for the brain to process, requiring sharper contrast to stand out. This correlation is essential in designing visual elements for accessibility, safety, and clarity, ensuring objects are easily recognized under various lighting and contrast conditions.

In some embodiments, the correlation between the object size and the required contrast level may be determined for each individual person during a vision test. The correlation may indicate a person's visual performance in practical situations, e.g., detecting road signs in foggy conditions or identifying objects in dim lighting, where size can compensate for reduced contrast, allowing recognition to occur more easily. Further, in some embodiments, each of the curves 2302 and 2304 shows an inverse relationship between the required contrast level and the size of the object. A user 120 who has a glaucoma condition requires a higher contrast level to recognize an object compared with a user having healthy eyes.

FIG. 24 is a flow diagram of an example vision test process 2400 for determining a contrast sensitivity profile 2414 of a user 120, in accordance with some embodiments. A computer device 140 (e.g., headset device 140D) may include an HMD 312A, and one or more cameras 310A (e.g., outward camera 378 and eye-tracking camera 366 in FIG. 3). The computer device 140 may execute a user application (e.g., a visual assessment application 328) configured to enable a virtual vision test and generate a VR user interface 2102 corresponding to a 3D virtual environment. The computer device 140 successively may display a plurality of visual stimuli 2402 corresponding to a plurality of acuity levels 2404 in a 3D virtual environment. At each acuity level 2404, the plurality of visual stimuli 2402 have a plurality of respective shadings. For example, the plurality of visual stimulus 2402 corresponding to a first acuity level may be displayed concurrently at a first time, and the plurality of visual stimulus 2402 corresponding to a second acuity level may be displayed concurrently at a second time subsequent to the first time. The computer device 104 obtains a plurality of user responses 2406 to the plurality of visual stimuli 2402. For each acuity level 2404, the computer device 104 may determine a respective contrast perception level 2408 corresponding to the respective acuity level 2404 based on the one or more user responses 2406. The computer device 104 may generate a contrast profile 2410 of a user 120 associated with the computer device 104, and the contrast profile 2410 may map the respective contrast perception level 2408 with respect to the respective acuity level 2404 for the plurality of acuity levels 2404.

In some embodiments, the plurality of acuity levels 2404 may correspond to a plurality of distances 2412. At each distance, the plurality of visual stimuli 2402 may have a respective optotype size, and a respective acuity level 2404 is defined based on the respective distance 2412 and the respective optotype size.

In some embodiments, the computer device 140 may generate a contrast sensitivity profile 2414 of the user 120 based on the contrast profile 2410. For each acuity level 2404, a corresponding contrast sensitivity may be determined based on a variation of the contrast perception level 2408 between the respective acuity level 2404 and a distinct acuity level that is closest to the respective acuity level 2404 among the plurality of acuity levels 2404. The contrast sensitivities of the plurality of acuity levels may be normalized to form the contrast sensitivity profile 2414 of the user 120.

In some embodiments, the computer device 140 may determine that the user has an eye disease condition 2416 (e.g., glaucoma, cataracts, and optic neuritis) based on the contrast profile 2410. Further, in some embodiments, the computer device 140 may determine a severity level 2418 of the eye disease condition 2416 based on the contrast profile 2410, the contrast sensitivity profile 2414, or both profiles. In some embodiments, the computer device 140 may compare the contrast profile 2410 with a reference contrast profile of a healthy eye to identify the eye disease condition 2416 and/or determine the associated severity level 2418. In some embodiments, the computer device 140 may apply a contrast profiling model 2420 to process the contrast profile 2410, the contrast sensitivity profile 2414, or both profiles to identify the eye disease condition 2416 and/or determine the associated severity level 2418.

FIG. 25 is a flow diagram of an example vision test process 2500 for controlling shadings of visual stimuli 2402 in a 3D virtual environment, in accordance with some embodiments. A computer device 140 (e.g., headset device 140D) may include an HMD 312A, and one or more cameras 310A (e.g., outward camera 378 and eye-tracking camera 366 in FIG. 3). The computer device 140 may execute a user application (e.g., a visual assessment application 328) configured to enable a virtual vision test and generate a VR user interface 2102 corresponding to a 3D virtual environment. The computer device 140 may display a plurality of visual stimuli 2402 (e.g., concurrently) at a first distance 2502A in the 3D virtual environment and obtain one or more user responses 2406. The plurality of visual stimuli 2402 may have a first optotype size 2504A and a plurality of first shadings 2506A, and the first distance 2502A and the first optotype size 2504A defines a first acuity level 2404A. Based on the one or more user responses 2406, the computer device 140 may determine a first contrast perception level 2408A corresponding to the first acuity level 2404A. The computer device 140 may further determine a shading range 2508 for a second acuity level 2404B based on the first contrast perception level 2408A. A plurality of second shadings 2506B may be further determined in the shading range 2508. By these means, the shading range 2508 is dynamically controlled (e.g., narrowed) to expedite the vision test process 2500, and the visual stimuli 2402 can be displayed in the shading range 2508 with a higher shading resolution without requesting the user 120 to review a large number of visual stimuli 2402.

The computer device 140 may display the plurality of visual stimuli 2402 (e.g., concurrently) at a second distance 2502B in the 3D virtual environment. The plurality of visual stimuli 2402 may have a second optotype size 2504B and the plurality of second shadings 2506B. In some embodiments, the first optotype size 2504A may be equal to the second optotype size 2504B, and the first distance 2502A may be different from the second distance 2502B. Alternatively, in some embodiments, the first optotype size 2504A may be different from the second optotype size 2504B, and the first distance 2502A may be equal to the second distance 2502B.

In some embodiments, given a correlation (FIG. 23) between a size of an object and a contrast level required for a person's eyes to recognize the object, when the first contrast perception level 2408A is determined, the first contrast perception level 2408A may be applied to set an upper limit of the shading range 2508 for the second acuity level 2404B that is lower than the first acuity level 2404A or to set a lower limit of the shading range 2508 for the second acuity level 2404B that is higher than the first acuity level 2404A. In some situations, the first contrast perception level 2408A may be equal to the upper or lower limit of the shading range 2508. In some situations, the first contrast perception level 2408A may be used to determine, but not equal to, the upper or lower limit of the shading range 2508.

In some embodiments, the computer device 140 may determine a contrast perception level 2408 corresponding to each of the second acuity level 2404B and one or more third acuity levels (not shown). A shading range 2508 may be determined for each of the one or more third acuity levels. For each of the one or more third acuity levels, the computer device may display the plurality of visual stimuli 2402 at a respective third distance 2502C in the 3D virtual environment.

In some embodiments, the computer device 140 may generate a contrast profile 2410 of a user 120 associated with the computer device 140. The contrast profile 2410 may map the contrast perception level 2408 with respect to a plurality of acuity levels 2404 including the first acuity level 2404A. Further, in some embodiments, the contrast profile 2410 may include a plurality of data pairs, and each data pair includes a respective contrast perception level and a respective acuity level. The plurality of data pairs may include a first data pair further including the first contrast perception level 2408A and the first acuity level 2404A. An example of the contrast profile 2410 may be represented by a curve 2302 or 2304 in FIG. 23.

In some embodiments, the computer device 140 may generate a contrast sensitivity profile 2414 of the user 120 based on the contrast profile 2410. Further, in some embodiments, the computer device 140 may determine that the user 120 has an eye disease condition 2416 (e.g., glaucoma, cataracts, or optic neuritis) based on the contrast profile 2410. Additionally, in some embodiments, the computer device 140 may obtain a plurality of reference profiles corresponding to a plurality of known eye conditions (e.g., corresponding to normal or impaired eyes), and compare the contrast profile 2410 of the user 120 with each of the plurality of reference profiles to identify the eye disease condition 2416. In some embodiments, the computer device 140 may compare the contrast profile 2410 of the user 120 with one or more reference contrast profiles of the eye disease condition 2416 to determine a severity level 2418 of the eye disease condition 2416.

In some embodiments, the computer device 140 may apply a contrast diagnosis model 2510 to process the contrast profile 2410 to determine that the user has the eye disease condition 2416. In some embodiments, the computer device 140 may apply a severity diagnosis model 2512 to determine a severity level 2418 of the eye disease condition 2416.

In some embodiments, the one or more user responses 2406 include a user input captured by one or more first sensors of the computer device 140. The one or more first sensors include a forward-facing camera 378 (FIG. 3) for detecting a hand gesture, a microphone 380 (FIG. 3) for collecting an audio response, or a controller 390 (FIG. 3) for receiving a user physical force. In some embodiments, the user response 1906 may include a spontaneous user response monitored by one or more second sensors of the computer device 140. The one or more second sensors include one or more of: an eye tracking camera 366, a heart rate sensor 2520, a body temperature sensor 2522, a blood oxygen level 2524, a Galvanic skin response sensor 2526, a hand gesture camera (e.g., camera 378), a body gesture camera (e.g., camera 378), a microphone 380, a motion sensor 376, and a set of one or more brain activity electrodes 362. More details on the one or more user responses 2406 are discussed above with reference to the user response 1906 in FIG. 19. In some embodiments, the user 120 may be prompted to use a controller 390 (FIG. 3) to identify one of the visual stimuli 2402 that can or cannot be visible to the user 120.

In some embodiments, the plurality of first shadings 2506A may correspond to a first shading resolution 2514A. The compute device 104 may determine a response time 2516 of the one or more user responses 2406. For example, the computer device 104 may obtain a sequence of eye images from which eye movement information is extracted automatically and without user intervention. When the visual stimuli 2402 are displayed, the computer device 140 may determine the response time 2516 based on a temporal sequence of eyeball positions extracted from the sequence of eye images. In accordance with a determination that the response time 2516 is greater than a response threshold, the computer device may determine a second shading resolution 2514B for the plurality of plurality of second shadings 2506B, and the second shading resolution 2514B is lower than the first shading resolution. Stated another way, in some embodiments, the first shading resolution 2514A may be excessively fine, and it may take an extended time for the user 120 to differentiate optotypes having close contrast levels. The second shading resolution 2514B is lower and may expedite the vision test process 2500.

In some embodiments, the plurality of visual stimuli 2402 displayed with different shadings 2506A may correspond to different optotypes, and the user 120 may be prompted to recognize individual optotypes. In some embodiments not shown, the plurality of visual stimuli 2402 displayed with different shadings 2506A may correspond to a single optotype (e.g., “E”). The user 120 may be prompt to identify which one of the visual stimuli 2402 starts to be invisible. In some embodiments, the visual stimuli 2402 are displayed concurrently and spatially arranged according to decreasing or increasing shadings. Alternatively, in some embodiments, the visual stimuli 2402 are displayed concurrently with random shadings within a respective shading range 2508.

In some embodiments, the plurality of visual stimuli 2402 are displayed on a background view to test the user's contrast perception under different conditions (e.g., exposed to different lightings or disturbances). The computer device 140 may render a static image or a stream of video data associated with the background view on the user interface. The plurality of visual stimuli 2402 may be overlaid on the static image or a set of respective image frames in the stream of video data associated with the background view. Further, in some embodiments, the background view is one of: a static beach view, a static city night scene, and a dynamic traffic view. In some embodiments, the background view includes a doctor's office where the vision test process 2500 is implemented. Stated another way, the vision test process 2500 is implemented in a 3D augmented reality environment.

### Adjustment of Illumination in Virtual Vision Tests for Drivers

Some implementations of this application include a VR-based computer system 300 configured to assess contrast vision of eyes using 3D high-contrast visual environments. The computer system 300 may utilize a high-resolution VR headset equipped with precision eye-tracking sensors (e.g., eye-tracking cameras 366 in FIG. 3) and specialized software algorithms to generate high-contrast visual scenarios. Users wear the VR headset and engage in a series of interactive tasks within virtual environments specifically configured to challenge their contrast vision. These environments include simulations of real-world scenarios such as night driving with headlights, navigating through shadowy areas with bright highlights, and identifying objects against varying backgrounds. The eye-tracking sensors may monitor the user's focus adjustments and visual acuity, while the software analyzes the user responses to evaluate contrast vision performance.

In some embodiments, the VR-based computer system 300 may incorporate a range of visual tasks, e.g., detecting objects in low-contrast settings, distinguishing between fine details in brightly lit and dark areas, which may require prompt adjustment of a user's eyes to changing lighting conditions. A user application 324 (e.g., visual assessment application 328 in FIG. 3) may process the data in real time and assess the user's ability to perceive contrasts and identify details in high-contrast environments. Results may be compiled into a detailed report that provides insights into the user's contrast vision capabilities, highlighting any deficiencies that may indicate underlying ocular conditions like glaucoma, diabetic retinopathy, or cataracts. The computer system 300 offers a non-invasive, engaging, and accurate approach to evaluate contrast vision in a variety of demanding visual contexts.

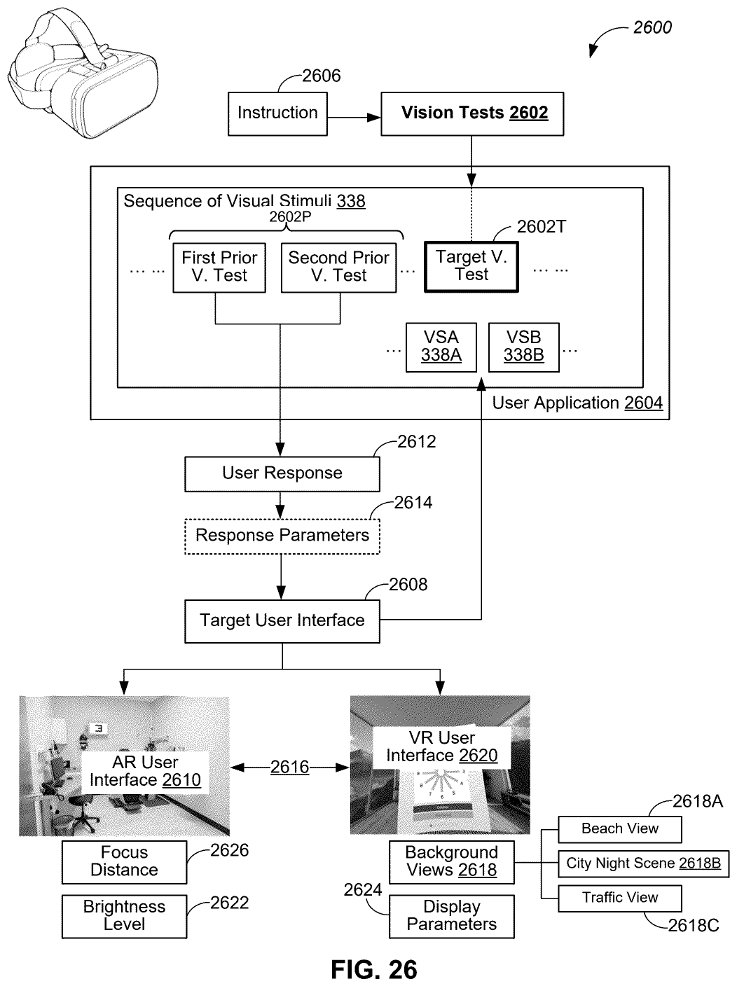

FIG. 26 is a flow diagram of an example process 2600 of selecting one of an AR user interface 2610 and a VR user interface 2620 to implement a vision test 2602, in accordance with some embodiments. The process 2600 may be implemented using a computer device 140 (e.g., headset device 140D), which may include an HMD 312A, one or more processors 302, and memory 306 (FIG. 3) storing instructions to be implemented by the processor(s) 302. The computer device 140 may execute a user application 2604 (e.g., a visual assessment application 328) configured to generate a target user interface 2608 corresponding to a 3D virtual environment and enable one or more virtual vision tests 2602 via the target user interface 2608. A sequence of visual stimuli 338 may correspond to the one or more virtual vision tests 2602 and be displayed on the target user interface 2608 successively. Each virtual vision test 2602 may include a subset of respective visual stimuli 338. More specifically, the computer device 140 may obtain an instruction 2606 to implement the target vision test 2602T, and select the target user interface 2608 for the target vision test 2602T between a VR user interface 2620 corresponding to a 3D VR environment and an AR user interface 2610 corresponding to a 3D AR environment. The target vision test 2602T on the target user interface 2608.

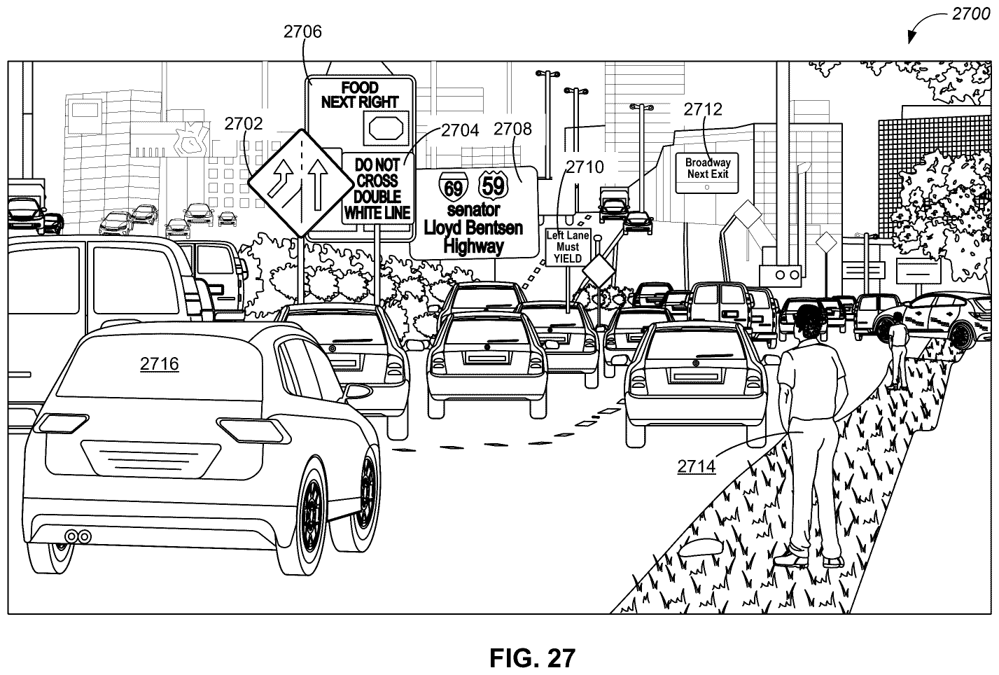

The VR user interface 2620 may provide an immersive environment that completely replaces the real world, transporting a user 120 wearing the HMD 312A to a simulated, interactive 3D VR environment (e.g., a traffic scene 2700 in FIG. 27). The computer device 140 may include the HMD 312A, hand controllers, and sensors 360 to track body movements. The user 120 may navigate through menus, interact with objects, and control the 3D VR environment using gestures, head movements, or handheld devices. The VR user interface 2620 may prioritize creating a seamless and engaging experience, with intuitive controls that make the 3D VR environment feel tangible and responsive. An AR user interface 2610 may overlay digital virtual elements onto the real world, enhancing the user's perception of a physical environment (e.g., a doctor's office). The AR user interface 2610 can be experienced through smartphones 104C, tablets 104B, or headset device 140D. The user 120 may interact with digital information and objects superimposed on their surroundings using touch screens, voice commands, or gestures. Digital virtual elements may be integrated smoothly with the real world, making information easily accessible and interactive without losing the context of the physical environment. This blend of the real and virtual worlds may aim to enrich the user's interaction with their surroundings, providing contextual information and enhancing real-world tasks.

In some embodiments, a sequence of vision tests 2602 may include the target vision test 2602T and one or more prior vision tests 2602P implemented prior to the target vision test 2602T. The computer device 140 may monitor user responses associated with the one or more prior vision tests 2602P. The target user interface 2608 may be automatically selected between the VR user interface 2620 and the AR user interface 2610 based on the user responses 2612. In some embodiments, the user response 2612 may include a user input captured by a forward-facing camera 378 (FIG. 3) for detecting a hand gesture and/or a microphone 380 (FIG. 3) for collecting an audio response. In some embodiments, the user response 2612 may include a spontaneous user response (e.g., a pupil size) monitored by one or more of: an eye tracking camera 366, a heart rate sensor, a body temperature sensor, a blood oxygen level, a Galvanic skin response sensor, a hand gesture camera (e.g., camera 378), a body gesture camera (e.g., camera 378), a microphone 380, a motion sensor 376, and a set of one or more brain activity electrodes 362.

Further, in some embodiments, the computer device 140 may determine one of a plurality of response parameters 2614 (e.g., a response rate, a success rate, and a confidence score) based on the user responses 2612 associated with the one or more vision tests, and the target user interface 2608 is automatically selected based on the one of the plurality of response parameters 2614. Additionally, in some embodiments, in accordance with a determination that one of the response rate, the success rate, and the confidence score is lower than a respective threshold, the computer device 140 may switch (operation 2616) from one of the VR user interface 2620 and the AR user interface 2610 to the other one of the VR user interface 2620 and the AR user interface 2610 (e.g., from the VR user interface 2620 to the AR user interface 2610, from the AR user interface 2610 and the VR user interface 2620).

In some embodiments, the VR user interface 2620 is selected, and a set of one or more first visual stimuli 338A on the VR user interface 2620 in the 3D virtual environment. Further, in some embodiments, the computer device 140 may select a background view 2618, render a stream of video data associated with the background view 2618 on the AR user interface, and overlay each first stimulus 338A on a set of respective image frames in the stream of video data associated with the background view 2618. The background view 2618 may be selected in response to receiving a user selection of the background view 2618 from a plurality of background options. In some embodiments, a sequence of vision tests includes the target vision test 2602T and one or more prior vision tests 2602P implemented prior to the target vision test 2602T. The computer device 140 may monitor user responses 2612 associated with the one or more prior vision tests, and the background view 2618 may be automatically selected from a plurality of virtual background options based on the user responses 2612. In some embodiments, the background view 2618 may be one of: a static beach view 2618A, a static city night scene 2618B, and a dynamic traffic view 2618C.

In some embodiments, the AR user interface 2610 may be selected, and a set of one or more second visual stimuli 338B are displayed on the AR user interface 2610 in the 3D AR environment. Further, in some embodiments, the computer device 140 may set the HMD 312A to be transparent and seen through to show a field of view, and each second stimulus 338B may be overlaid on the field of view. Alternatively, in some embodiments, a forward-facing camera 378 (FIG. 3) of the computer device 140 may capture a stream of video data of a field of view. The stream of video data is rendered on the AR user interface 2610 in real time. Each second stimulus 338B may be overlaid on a set of respective image frames in the stream of video data. Additionally, in some embodiments, for each second visual stimulus 338B, the computer device 140 may determine a focus distance 2626 associated with the respective second visual stimulus 338B, and the respective second visual stimulus 338B is rendered at the focus distance 2626 on the AR user interface 2610. In some embodiments, the computer device 140 may adjust a brightness level 2622 of the AR user interface 2610, thereby testing the user's visual capability under different light conditions.

FIG. 27 is an example traffic scene 2700 enabled in a virtual environment for one or more vision tests 2602, in accordance with some embodiments. A computer device 140 includes an HMD 312A, one or more processors 302, and memory 306 (FIG. 3). The computer device 140 may execute a user application 2604 configured to enable the one or more vision tests 2602. For example, one or more vision tests 2602 are set in the traffic scene 2700, and the user application 2604 is configured to execute the vision test 2602 and facilitate issuance or update of a driver license. The computer device 140 may obtain an instruction 2606 to implement a target vision test 2602T. In accordance with a determination that the target vision test 2602T corresponds to a driver license issuing requirement, loading a VR user interface 2620 to create a 3D VR environment. The VR user interface 2620 includes the virtual traffic scene 2700, displaying a plurality of traffic signs 2702-2712 at a plurality of distances.

In some embodiments, the computer device 140 may display a plurality of traffic related objects in the virtual traffic scene 2700, the traffic related objects including one or more of: a traffic light, a pedestrian 2714, and a car 2716. At least one of the traffic related objects may be moving in the virtual traffic scene. When a user associated with the HMD 312A takes the target vision test 2602T, his or her visual capabilities (e.g., visual acuity, red and green traffic light recognition, visual response time) are tested in a dynamic traffic environment, allowing a government agency (e.g., Department of Motor Vehicle (DMV)) to issue driver licenses in a more reliable manner.

Referring to FIG. 27, in an example, the traffic signs 2702, 2704, 2706, 2708, 2710, and 2712 are arranged at increasing distances. Each traffic sign is displayed with a set of respective display parameters 2624 (FIG. 26), such as a font size, a foreground color, a brightness level, and a background style. The user associated with the HMD 312A takes the target vision test 2602T may be prompted to identify what is displayed on each traffic sign. In some embodiments, a light condition of the virtual traffic scene 2700 is adjusted to test whether the user may still recognize what is displayed on each traffic sign. For example, the light condition may correspond to a sunset time, and the user may be prompted to recognize what is displayed on each traffic sign. In some embodiments, the user having green-red color blindness may be prompted to indicate whether a color of a traffic light is green or red at a sunset time. Based on the user's responses, it may be determined whether the user's color blindness level reaches a severity level that may cause a traffic accident.

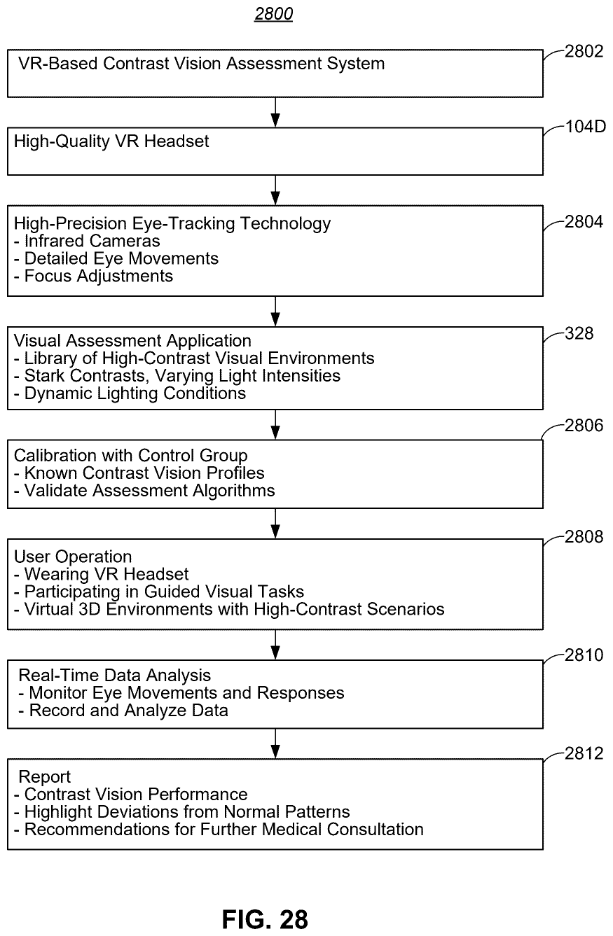

FIG. 28 is a flow diagram of an example vision test process 2800 for controlling an illumination scheme 2908 in a 3D virtual environment, in accordance with some embodiments. The VR-based computer system 300 may enable a VR-based contrast vision assessment system 2802. The computer system 300 may include a VR headset 104D that includes an eye-tracking camera 366 (FIG. 3). The eye-tracking technology 2804 may include an infrared camera (e.g., camera 366) configured to capture eye movements and focusing adjustments. In some embodiments, when a visual assessment application 328 is executed, a library of high-contrast visual environments is applied to test different aspects of contrast vision. The visual environments may include scenarios with stark contrasts, varying light intensities, and dynamic lighting conditions to simulate real-world visual challenges.

In some embodiments, when hardware components and software modules may be integrated to form the VR-based contrast vision assessment system, the VR-based computer system 300 may be calibrated (operation 2806) using a control group of individuals with known contrast vision profiles, thereby establishing baseline performance metrics and validate the accuracy of the assessment algorithms. Users can operate (operation 2808) the system by wearing the VR headset and participating in the guided visual tasks within the virtual environments. The eye-tracking camera 366 may monitor focus adjustments and visual responses of a user's eyes to the high-contrast visual scenarios. Image or video data recorded by the camera 366 may be analyzed (operation 2810) in real time by the software modules (e.g., visual assessment application 328, data processing module 330 in FIG. 3). In some implementations, the user may receive a report 2812 outlining associated contrast vision performance, highlighting deviations from normal patterns, and providing recommendations for further medical consultation if necessary. By these means, the computer system 300 may offer a precise, non-invasive, and user-friendly method for assessing contrast vision, representing a significant advancement over traditional testing techniques, and providing substantial benefits for both clinical and consumer applications.

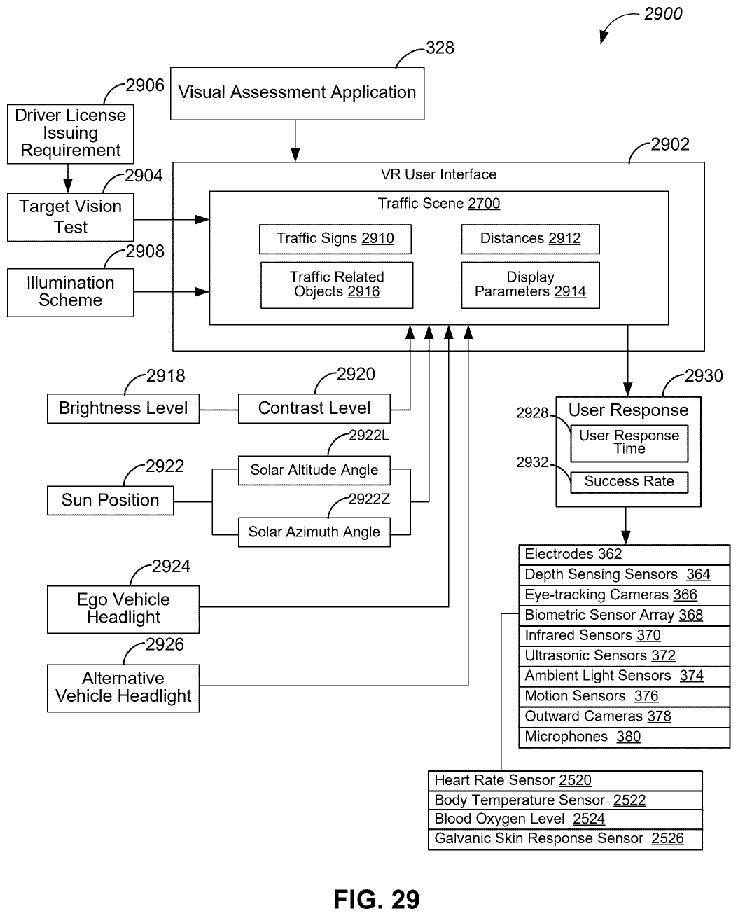

FIG. 29 is a flow diagram of an example vision test process 2900 for assessing a contrast sensitivity level of a user's eyes, in accordance with some embodiments. A computer device 140 (e.g., headset device 140D) may include an HMD 312A, and one or more cameras 310A (e.g., outward camera 378 and eye-tracking camera 366 in FIG. 3). The computer device 140 may execute a user application (e.g., a visual assessment application 328) configured to enable the virtual vision test and generate a VR user interface 2902 corresponding to a 3D virtual environment. The computer device 140 may obtain an instruction to implement a target vision test 2904. In accordance with a determination that the target vision test 2904 corresponds to a driver license issuing requirement 2906, the computer device 104 may load the VR user interface 2902, determine an illumination scheme 2908, and display a virtual traffic scene 2700 (FIG. 27) on the VR user interface 2902 based on the illumination scheme 2908. The virtual traffic scene 2700 may include a plurality of traffic signs 2910 (e.g., signs 2702-2712 in FIG. 27) located at a plurality of distances 2912.

In some embodiments, each traffic sign 2910 may be displayed with a set of display parameters 2914 including a sign size. In some embodiments, a plurality of traffic related objects 2916 may be in the virtual traffic scene 2700. The traffic related objects 2916 may include one or more of: a traffic light, a pedestrian 2714, and a car 2716. At least one of the traffic related objects 2916 may be moving in the virtual traffic scene 2700.

In some embodiments, the illumination scheme 2908 may correspond to a brightness level 2918 and a contrast level 2920, and be uniformly applied to the virtual traffic scene 2700. Alternatively, in some embodiments, the illumination scheme 2908 may corresponds to a sun position 2922. In accordance with the illumination scheme 2908, the virtual traffic scene 2700 may be adaptively rendered based on the sun position 2922. Further, in some embodiments, the sun position 2922 may include a solar altitude angle (Alt) 2922L and a solar azimuth angle (Az) 2922Z. In some embodiments, a sun-based scene rendering model (e.g., a generative neural network) may be applied to render the virtual traffic scene 2700 based on the sun position 2922.

In some embodiments, the illumination scheme 2908 may correspond to an ego vehicle headlight 2924. A local portion of the virtual traffic scene 2700 may be in proximity to a user 120 associated with the computer device 140, and at least one of the plurality of traffic signs 2910 may be exposed to illumination of the ego vehicle headlight 2924. In some embodiments, the illumination scheme 2908 corresponds to one or more alternative vehicle headlights 2926. One or more local areas of the virtual traffic scene 2700 may be illuminated based on locations of the one or more alternative vehicle headlights 2926. At least one of the plurality of traffic signs 2910 may be exposed to illumination of the one or more alternative vehicle headlights 2926.

In some embodiments, while displaying the plurality of traffic signs 2910 on the virtual traffic scene 2700, the computer device 140 may monitor a user response 2930 to each of a subset of traffic signs 2910. In some embodiments, the one or more user responses 2406 include a user input captured by one or more first sensors of the computer device 140. The one or more first sensors include a forward-facing camera 378 (FIG. 3) for detecting a hand gesture, a microphone 380 (FIG. 3) for collecting an audio response, or a controller 390 (FIG. 3) for receiving a user physical force. In some embodiments, the user response 1906 may include a spontaneous user response monitored by one or more second sensors of the computer device 140. The one or more second sensors include one or more of: an eye tracking camera 366, a heart rate sensor 2520, a body temperature sensor 2522, a blood oxygen level 2524, a Galvanic skin response sensor 2526, a hand gesture camera (e.g., camera 378), a body gesture camera (e.g., camera 378), a microphone 380, a motion sensor 376, and a set of one or more brain activity electrodes 362. More details on the one or more user responses 2406 are discussed above with reference to the user response 1906 in FIG. 19.

In some embodiments, the computer device 104 may obtain a sequence of eye images from which eye movement information is extracted automatically and without user intervention. When the visual stimuli 2402 are displayed, the computer device 140 may determine the response time 2516 based on a temporal sequence of eyeball positions extracted from the sequence of eye images. The computer device 140 may determine a response time of the user response 2930 associated with a first traffic sign (e.g. traffic sign 2712 in FIG. 27). In accordance with a determination that the response time 2928 is greater than a response threshold, the computer device 140 may adjust the illumination scheme 2908 to update the plurality of traffic signs 2910 on the virtual traffic scene 2700.

In some embodiments, the computer device 104 may determine a current success rate 2932 for the subset of traffic signs based on the one or more user response 2930. In accordance with a determination that the current success rate 2932 is lower than a failure threshold, the computer device 140 may adjust the illumination scheme 2908 to update the plurality of traffic signs 2910 on the virtual traffic scene 2700.

Some implementations of this application are directed to implementing a traffic vision test, e.g., as part of a procedure for getting or updating a driver's license. A computer device 140 (e.g., headset device 140D) may include an HMD 312A, and one or more cameras 310A (e.g., outward camera 378 and eye-tracking camera 366 in FIG. 3). The computer device 140 may execute a user application (e.g., a visual assessment application 328) configured to enable the virtual vision test and generate a VR user interface 2902 corresponding to a 3D virtual environment. The computer device 140 may display a plurality of traffic signs 2910 at a plurality of distances on a virtual traffic scene 2700 and apply an illumination scheme 2908 to the virtual traffic scene 2700. In some embodiments, the illumination scheme 2908 may correspond to a brightness level and a contrast level, and is uniformly applied to the virtual traffic scene. In some embodiments, the illumination scheme 2908 may correspond to a sun position 2922 (e.g., a solar altitude angle 2922L and a solar azimuth angle 2922Z). In some embodiments, the illumination scheme 2908 may correspond to an ego vehicle headlight 2924. A local portion of the virtual traffic scene 2700 may be in proximity to a user 120 associated with the computer device 140, and at least one of the plurality of traffic signs 2910 may be exposed to illumination of the ego vehicle headlight 2924. In some embodiments, the illumination scheme 2908 corresponds to one or more alternative vehicle headlights 2926. One or more local areas of the virtual traffic scene 2700 may be illuminated based on locations of the one or more alternative vehicle headlights 2926, and at least one of the plurality of traffic signs 2910 may be exposed to illumination of the one or more alternative vehicle headlights 2926.

### Depth Perception Scanning Along Lines of Sight

Some implementations of this application include a VR-based computer system 300 configured to simulate depth perception challenges to eyes using stereo vision tests. The computer system 300 may utilize a high-resolution VR headset equipped with precision eye-tracking sensors (e.g., eye-tracking cameras 366 in FIG. 3) and specialized software algorithms to generate stereoscopic visual stimuli. Users wear the VR headset and participate in a series of interactive tasks that involve perceiving and responding to 3D objects and environments. These tasks are specifically configured to challenge and evaluate various aspects of depth perception, including binocular disparity, stereopsis, and spatial judgment. The eye-tracking sensors may monitor the user's focus adjustments and visual acuity, while the software analyzes the user responses to provide a comprehensive assessment of the user's depth perception capabilities.

In some embodiments, the VR-based computer system 300 may implement a range of stereoscopic vision tests, such as matching the relative distances of objects, navigating through 3D mazes, and identifying the depth of different elements in a virtual scene. The visual assessment application 328 (FIG. 3) may dynamically adjust the complexity of the stereoscopic vision tests based on the user's performance, ensuring a personalized assessment experience. The collected data may be processed in real time using advanced algorithms (e.g., machine learning models) to measure the user's ability to integrate visual information from both eyes to perceive depth information. Results may be compiled into a report that provide information regarding the user's depth perception strengths and weaknesses, and offers insights for diagnosing the user's visual conditions (e.g., strabismus, amblyopia, or convergence insufficiency).

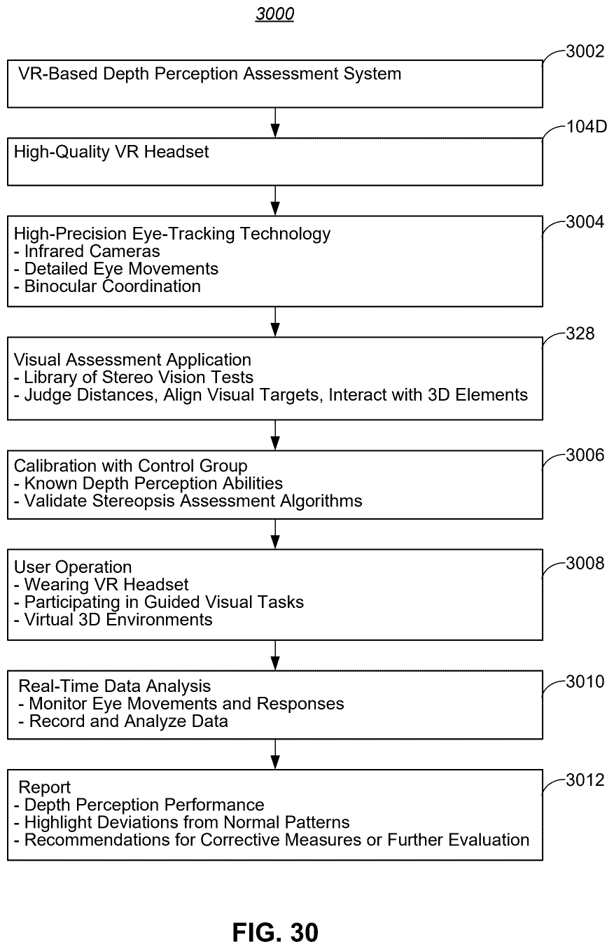

FIG. 30 is a flow diagram of an example vision test process 3000 for assessing a depth perception level of a user's eyes, in accordance with some embodiments. The VR-based computer system 300 is configured to enable a VR-based depth perception assessment system 3002. The computer system 300 may include a VR headset 104D that includes an eye-tracking camera 366 (FIG. 3). The eye-tracking technology 3004 may include an infrared camera (e.g., camera 366) configured to capture eye movements and binocular coordination. In some embodiments, when a visual assessment application 328 is executed, a library of stereo vision tests is applied to evaluate different aspects of depth perception. These tests may include scenarios where users may be prompted to differentiate objects at different distances, align visual targets, and interact with 3D elements in a virtual space.

In some embodiments, when hardware components and software modules may be integrated to form the VR-based depth perception assessment system, the VR-based computer system 300 may be calibrated (operation 3006) using a control group of individuals with known depth perception abilities, thereby establishing baseline performance metrics and validate the accuracy of the stereopsis assessment algorithms. Users can operate (operation 3008) the system by wearing the VR headset 104D and participating in the guided stereo vision tests within the virtual environments. The eye-tracking sensors may monitor eye movements and user responses to the 3D stimuli. Image or video data recorded by the camera 366 may be analyzed (operation 3010) in real time by the software modules (e.g., visual assessment application 328, data processing module 330 in FIG. 3). In some implementations, the user may receive a report 3012 outlining depth perception performance, highlighting deviations from normal patterns, and providing recommendations for corrective measures or further medical consultation. By these means, the computer system 300 may offer a precise, non-invasive, and user-friendly method for assessing depth perception, representing a significant advancement over traditional refractive assessment techniques, and providing substantial benefits for both clinical and consumer applications.

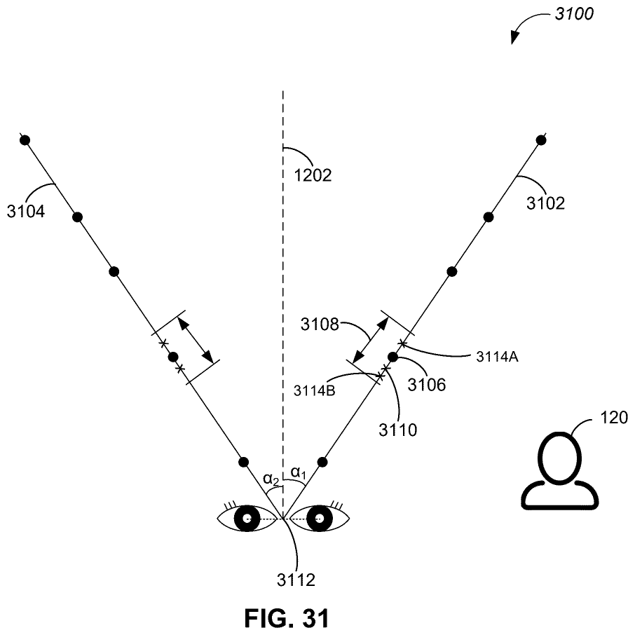

FIG. 31 is a diagram 3100 of two example lines of sight 3102 and 3104 associated with a binocular vision of a user 120, in accordance with some embodiments. A headset device 140D may execute a user application (e.g., a visual assessment application 328) configured to enable a virtual vision test and generate a VR user interface corresponding to a 3D virtual environment. A line of sight (e.g., lines 3102 or 3104) may correspond to a straight unobstructed path between a user 120 wearing the headset device 140D and a location in the 3D virtual environment, where the location is occupied by an object or corresponds to a remote point. A line of sight may also be called visual axis, sightline, and sight line. The line of sight is an imaginary line between the user's eyes and a subject of interest. In some embodiments, the visual assessment application 328 may display a visual stimulus at a location on the line of sight 3102 or 3104. When the user 120 faces and looks forward, the line of sight 1202 may be perpendicular to a line connecting two eyeballs and presumed to have an angle of 0 degree. The line of sight 3102 has a first angle α₁ with respect to the line of sight 1202, and the line of sight 3104 has a second angle α₂ with respect to the line of sight 1202.

In some embodiments, each of the lines of sight 3102 or 3104 has a plurality of positions 3106, and each position 3106 is located in a respective position range 3108. For each position 3106, an object may be displayed at different locations 3110 within the respective position range 3108. While the object is displayed within the respective position range 3108, a plurality of user responses may be monitored and applied to determine a depth perception level of the user 120 associated with a corresponding line of sight 3102 or 3104.

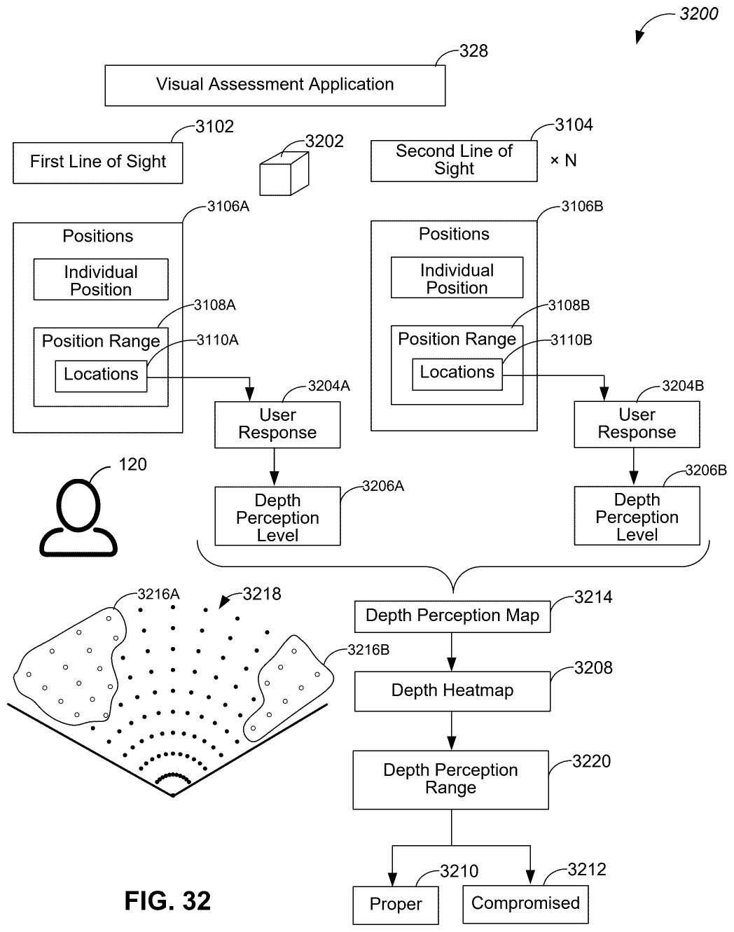

FIG. 32 is a flow diagram of an example vision test process 3200 for determining a depth perception range 3220 of a user 120, in accordance with some embodiments. A computer device 140 (e.g., headset device 140D) may include an HMD 312A, and one or more cameras 310A (e.g., outward camera 378 and eye-tracking camera 366 in FIG. 3). The computer device 140 may execute a user application (e.g., a visual assessment application 328) configured to enable the virtual vision test and generate a VR user interface 2902 corresponding to a 3D virtual environment. The computer device 140 may identify a first line of sight 3102 of a user 120 associated with the computer device 140. A plurality of positions 3106 may be selected on the first line of sight 3102, and each position 3106A may be located in a respective position range 3108A. For each position 3106A, the computer device 140 may display an object 3202 at a plurality of locations 3110A within the respective position range 3108A. The computer device 140 may obtain a plurality of user responses 3204A to displaying the object 3202 for each position 3106A. Based on the plurality of user responses 3204A, the computer device 140 may determine a depth perception level 3206A of the user 120 associated with the first line of sight 3102.

In some embodiments, the computer device 104 may identify a standard line of sight 1202 (FIG. 31) that extends forward from a center 3112 of, and is perpendicular to, a line connecting two eyes of the user 120. The first line of sight 3102 has a first angle α₁ with respect to the standard line of sight 1202. Further, in some embodiments, the first line of sight 3102 may be the standard line of sight 1202, and the first angle α₁ is equal to 0. In some embodiments, the first angle α₁ is within a predefined binocular angular range (e.g., (e.g., [−60°, 60°]) corresponding to a binocular area 1804 in an HFOV 1800 (FIG. 18). The computer device 104 may determinate that the depth perception level 3206A is lower than a reference depth level, which indicates that the user's depth perception associated with his binocular vision is compromised.

In some embodiments, the depth perception level 3206A includes a plurality of depth sensitivity levels. Fr each position 3106 on the first line of sight 3102, the computer device 140 may determine a respective depth sensitivity level based on a subset of user responses. Further, in some embodiments, for each position 3106A, based on the plurality of user responses 3204A, the computer device 140 may identify two of the plurality of locations (e.g., locations 3114A and 3114B in FIG. 31) that are differentiated by the user 120 and have a distance that is smaller than distances between any other two of the plurality of locations 3110A. The respective sensitivity level may include the distance of the two of the plurality of locations (e.g., locations 3114A and 3114B in FIG. 31).

In some embodiments, the computer device 104 may identify one or more second lines of sight 3104. For each second line of sight 3104, the computer device 104 may determine a respective depth perception level 3206B, thereby forming a depth perception map 3214 associating the first line of sight 3102 and the one or more second lines of sight 3104. Further, in some embodiments, the computer device 140 may generate a depth heatmap 3208 indicating a region having respective depth perception levels 3206A or 3206B within a normal range. In some embodiments, for each second line of sight 3104, the computer device 140 may select a plurality of second positions 3106B on the second line of sight 3104. Each second position 3106B may be located in a respective second position range 3108B. For each second position 3106B, the object may be displayed at a plurality of second locations 3110B within the respective second position range 3108B. The computer device 140 may obtain a plurality of second user responses 3204 to displaying the object 3202 for each second position 3106. Based on the plurality of second user responses 3204B, the computer device 140 may determine the depth perception level 3206B of the user 120 associated with the second line of sight 3104.

Further, in some embodiments, the computer device may compare the depth perception map 3214 with a reference perception map to determine whether the user's depth perception is compromised. For example, a similarity level may be determined between the depth perception map 3214 and the reference perception map. In accordance with a determination that the similarity level reaches or is above a similarity threshold, the user's depth perception is determined to be proper 3210. Conversely, in accordance with a determination that the similarity level is below the similarity threshold, the user's depth perception is determined to be impaired 3212.

Additionally, in some embodiments, the computer device 140 may determine a depth perception range 3220 of the user 120 based on the depth perception map 3214. The depth perception range 3220 is compared to a predefined binocular angular range (e.g., from −60 degrees to 60 degrees). Further, in some embodiments, in accordance with a determination that the depth perception range 3220 includes the predefined binocular angular range, the computer device 140 determine that the depth perception level 3206 of the user 120 is proper 3210. Conversely, in some embodiments, in accordance with a determination that the depth perception range 3220 misses a subset of the predefined binocular angular range, the computer device may determine that the depth perception level 3206 of the user is compromised 3212. For an example depth heatmap 3208, low depth perception levels may be observed at a first area 3216A and a second area 3216B.

In some embodiments, the plurality of user responses 3204A may include a user input captured by one or more first sensors of the computer device 140. The one or more first sensors include a forward-facing camera 378 (FIG. 3) for detecting a hand gesture, a microphone 380 (FIG. 3) for collecting an audio response, or a controller 390 (FIG. 3) for receiving a user physical force. In some embodiments, the plurality of user responses 3204A may include a spontaneous user response monitored by one or more second sensors of the computer device 140. The one or more second sensors include one or more of: an eye tracking camera 366, a heart rate sensor, a body temperature sensor, a blood oxygen level, a Galvanic skin response sensor, a hand gesture camera (e.g., camera 378), a body gesture camera (e.g., camera 378), a microphone 380, a motion sensor 376, and a set of one or more brain activity electrodes 362. More details on the one or more user responses 2406 are discussed above with reference to the user response 1906 in FIG. 19. In some embodiments, the user 120 may be prompted to use a controller 390 (FIG. 3) to the object 3202 displayed at different locations 3110 associated with a plurality of positions 3106 on the lines of sight 3102 and 3104.

In some embodiments, the computer device 104 may obtain a sequence of eye images from which eye movement information is extracted automatically and without user intervention. When the object 3202 are displayed on different locations of the line of sight 3102 or 3104, the computer device 140 may determine the response time based on a temporal sequence of eyeball positions extracted from the sequence of eye images. The computer device 140 may determine a response time of the user response 3204A or 3204B associated with the object 3202, and adjust the depth perception level 3206A or 3206B of the user 120 based on the response time. In some embodiments, the computer device 104 may determine a current success rate associated with displaying of the object 3202, and adjust the depth perception level 3206A or 3206B of the user based on the current success rate.

Some implementations of this application are directed to implementing a vision test associated with a user's depth perception in a 3D virtual environment. The vision test may be managed based on lines of sight in an HFOV 1800 of the user 120. A computer device 140 (e.g., headset device 140D) may include an HMD 312A, and one or more cameras 310A (e.g., outward camera 378 and eye-tracking camera 366 in FIG. 3). The computer device 140 may execute a user application (e.g., a visual assessment application 328) configured to enable the virtual vision test and generate a VR user interface 2902 corresponding to a 3D virtual environment. The computer device 140 may identify a plurality of lines of sight (e.g., lines 3102 and 3104 in FIG. 31) of the user associated with the computer device 140, and generate a depth perception map 3214 (e.g., map 3218) associated with the plurality of lines of sight. A depth perception range 3220 of the user 120 may be determined based on the depth perception map 3214. Further, in some embodiments, the computer device 140 may identify a standard line of sight 1202 that extends forward from a center 3112 of, and is perpendicular to, a line connecting two eyes of a user 120. Each line of sight 3102 or 3104 has a respective angle with respect to the standard line of sight 1202. Additionally, in some embodiments, the depth perception map 3214 includes a plurality of depth perception levels 3206A and 3206B corresponding to a plurality of positions 3106 on at least one of the plurality of lines of sight 3102 and 3104.

### Illustration of the Subject Technology As Clauses

Various examples of aspects of the disclosure are described as numbered clauses (1, 2, 3, etc.) for convenience. These are provided as examples, and do not limit the subject technology. Identifications of the figures and reference numbers are provided below merely as examples and for illustrative purposes, and the clauses are not limited by those identifications.

Clause 1. A method for testing vision, comprising: at an electronic device including a head-mounted display (HMD): executing a visual assessment application, including generating a user interface corresponding to a three-dimensional (3D) virtual environment; displaying a visual stimulus at a target location in the 3D virtual environment; monitoring a head orientation of a user wearing the electronic device; and dynamically adjusting the target location of the visual stimulus based on the head orientation.

Clause 2. The method of Clause 1, further comprising: identifying a standard line of sight that extends forward from a center of, and is perpendicular to, a line connecting two eyes of the user; and selecting the target location on the standard line of sight.

Clause 3. The method of Clause 1 or 2, wherein the target location corresponds to a first orientation, and dynamically adjusting the target location of the visual stimulus further comprises: determining that the head orientation has stabilized at a current orientation distinct from the first orientation for a first extended duration of time; and in accordance with a determination with the first extended duration of time is greater than an orientation threshold, moving the target location to follow the current orientation.

Clause 4. The method of any of Clauses 1-3, further comprising: monitoring an eye position based on eye images captured by an eye-tracking camera, wherein the target location of the visual stimulus is dynamically adjusted based on both the head orientation and the eye position.

Clause 5. The method of Clause 4, wherein the target location corresponds to a first line of sight, the method further comprising: determining that the eye position has stabilized along a current line of sight distinct from the first line of sight for a second extended duration of time; and in accordance with a determination with the second extended duration of time is greater than a sight line threshold, moving the target location to follow the current line of sight.

Clause 6. The method of any of Clauses 1-5, further comprising: obtaining a motion signal measured by a motion sensor, wherein the motion sensor includes one or more of: an accelerometer and a gyroscope, and the head orientation is determined based on the motion signal.

Clause 7. The method of any of Clauses 1-6, further comprising: receiving a user response indicating whether the visual stimulus is clear to the user, wherein the user response includes a user input captured by one or more first sensors of the electronic device, and the one or more first sensors include a forward-facing camera for detecting a hand gesture, a microphone for collecting an audio response, and a controller for receiving a user physical force.

Clause 8. The method of any of Clauses 1-6, further comprising: receiving a user response indicating whether the visual stimulus is clear to the user, wherein the user response include a spontaneous user response monitored by one or more second sensors of the electronic device, and the one or more second sensors include one or more of: an eye tracking camera, a heart rate sensor, a body temperature sensor, a blood oxygen level, a Galvanic skin response sensor, a hand gesture camera, a body gesture camera, a microphone, a motion sensor, and a set of one or more brain activity electrodes; and based on the user response, automatically, determining whether the visual stimulus is clear to the user.

Clause 9. The method of any of Clauses 1-6, wherein the visual stimulus has a first stimulus size at the target location, further comprising: while keeping the first stimulus size, displaying the visual stimulus at one or more alternative locations that are different from the target location; and receiving a user response indicating that the visual stimulus starts to be clear at the target location to the user compared with the one or more alternative locations.

Clause 10. The method of any of Clauses 1-6, wherein the visual stimulus has a first stimulus size at the target location, further comprising: while displaying the visual stimulus at the target location, varying a size of the visual stimulus to one or more alternative stimulus sizes; and receiving a user response indicating that the visual stimulus starts to be clear at the first stimulus size to the user compared with the one or more alternative stimulus sizes.

Clause 11. The method of any of Clauses 1-9, further comprising: determining a target distance between the target location and the user; and determining a spherical power based on the target distance and the first stimulus size.

Clause 12. A method for testing vision, comprising: at an electronic device including a head-mounted display (HMD): executing a visual assessment application, including generating a user interface corresponding to a three-dimensional (3D) virtual environment; selecting a first line of sight including a plurality of locations; and displaying a visual stimulus having a fixed stimulus size successively on the plurality of locations in the 3D virtual environment; monitoring a head orientation of a user wearing the electronic device; and dynamically, adjusting a location of the visual stimulus based on the head orientation to keep the visual stimulus on the first line of sight.

Clause 13. The method of Clause 12, further comprising: identifying a standard line of sight that extends forward from a center of, and is perpendicular to, a line connecting two eyes of the user, wherein the first line of sight has a predefined first angle with respect to the standard line of sight.

Clause 14. The method of Clause 12 or 13, wherein the first line of sight corresponds to a first orientation, and dynamically adjusting the location of the visual stimulus further comprises: determining that the head orientation has stabilized at a current orientation distinct from the first orientation for a first extended duration of time; and in accordance with a determination with the first extended duration of time is greater than an orientation threshold, moving the location of the visual stimulus to follow the current orientation.

Clause 15. A method for testing vision, comprising: displaying a video clip including a plurality of image frames, each image frame including a predefined visual stimulus having a respective orientation with respect to a focal point; while displaying the video clip, obtaining eye image data of an eye of a user; collecting or extracting eye response data including a pupil size from the eye image data; determining a spontaneous user response to the video clip based on eye response data; and automatically determining one or more astigmatism parameters based on the spontaneous user response.

Clause 16. The method of Clause 15, wherein the eye image data include a plurality of eye images of the eye, and each eye image corresponds to a respective pupil size value, and wherein the pupil size varies with the plurality of eye images.

Clause 17. The method of Clause 16, further comprising: applying a pupil size extraction model to process each eye image and determine the respective pupil size value corresponding to the respective eye image.

Clause 18. The method of Clause 16 or 17, wherein the one or more astigmatism parameters include an astigmatism axis of the eye, the method further comprising: applying a pupil astigmatism model to process the pupil size that varies with the plurality of eye images and determine at least the astigmatism axis.

Clause 19. The method of Clause 18, wherein the one or more astigmatism parameters include a cylindrical power of the eye, and the pupil astigmatism model is applied to process the pupil size and determine the cylindrical power in addition to the astigmatism axis.

Clause 20. The method of Clause 15, wherein the one or more astigmatism parameters include at least one of a cylindrical power and an astigmatism axis of the eye, the method further comprising: applying a pupil astigmatism model to process the pupil size and determine the one or more astigmatism parameters.

Clause 21. The method of Clause 20, further comprising, at a server: obtaining an astigmatism parameter ground truth and a set of samples of a pupil size; training the pupil astigmatism model based on the set of samples of the pupil size and the astigmatism parameter ground truth; and providing the pupil astigmatism model to the electronic device.

Clause 22. The method of any of Clauses 15-21, wherein the predefined visual stimulus includes at least a straight-line segment that is aligned with the focal point.

Clause 23. The method of any of Clauses 15-21, wherein the predefined visual stimulus includes two straight-line segments that are aligned with the focal point, and the two straight-line segments are symmetric with each other with respect to the focal point.

Clause 24. The method of any of Clauses 15-21, wherein the predefined visual stimulus includes at least a first set of two or more straight-line segments that are closely disposed and parallel to each other, and each line segment is symmetric to a distinct line segment with respect to the focal point.

Clause 25. The method of any of Clauses 15-21, wherein the predefined visual stimulus includes two identical sets of two or more straight-line segments, and the two identical sets of line segments are symmetric to each other with respect to the focal point.

Clause 26. The method of any of Clauses 15-25, wherein the predefined visual stimulus is displayed at a distance and rotates continuously with respect to the focal point in the video clip for a plurality of cycles.

Clause 27. The method of Clause 26, wherein the predefined visual stimulus has a rotation speed that is below a threshold speed, and the plurality of cycles include a number of cycles that is within a range of cycle numbers.

Clause 28. The method of Clause 27, wherein the threshold speed is 5 cycles per minute, and the range of cycle numbers is 2-10 inclusively.

Clause 29. The method of any of Clauses 15-25, wherein the predefined visual stimulus is displayed at a distance and rotate with respect to the focal point in the video clip based on a plurality of discrete angular positions.

Clause 30. The method any of Clauses 15-29, further comprising: executing a visual assessment application, including generating a user interface corresponding to a three-dimensional (3D) virtual environment, wherein the video clip is displayed on the user interface.

Clause 31. A method for testing vision, comprising: displaying a video clip including a plurality of image frames, each image frame including a predefined visual stimulus having a respective orientation with respect to a focal point, such that the predefined visual stimulus is displayed rotating continuously with respect to the focal point in the video clip; while displaying the video clip, obtaining eye image data of an eye of a user; determining a user response to the video clip based on the eye image data; and automatically determining one or more astigmatism parameters based on the user response.

Clause 32. The method of Clause 31, wherein the user response includes a pupil size, the method further comprising: applying a pupil size extraction model to process each eye image and determine a respective pupil size value corresponding to the respective eye image.

Clause 33. The method of Clause 32, wherein the one or more astigmatism parameters include an astigmatism axis of the eye, the method further comprising: applying a pupil astigmatism model to process the pupil size that varies with a plurality of eye images of the eye image data and determine at least the astigmatism axis.

Clause 34. The method of Clause 33, wherein the one or more astigmatism parameters include a cylindrical power of the eye, and the pupil astigmatism model is applied to process the pupil size and determine the cylindrical power in addition to the astigmatism axis.

Clause 35. A method for testing vision, comprising: at an electronic device including a head-mounted display (HMD): executing a visual assessment application, including generating a user interface corresponding to a three-dimensional (3D) virtual environment; displaying a first visual stimulus at a first depth in the user interface, the first depth measured on a first line of sight; displaying the first visual stimulus at a second depth in the user interface, the second depth distinct from the first depth and measured on the first line of sight; obtaining one or more user responses to displaying of the first visual stimulus; and based on the one or more user responses, determining a depth perception level for a user associated with the electronic device.

Clause 36. The method of Clause 35, wherein the first stimulus is displayed on the first depth and the second depth concurrently.

Clause 37. The method of Clause 35, wherein the first stimulus is displayed on the first depth and the second depth sequentially.

Clause 38. The method of Clause 35, 36, or 37, further comprising: presenting a prompt requesting the user to indicate whether the user can visually differentiate the first depth from the second depth.

Clause 39. The method of any of Clauses 35-38, wherein the HMD includes a left display associated with a left eye of the user and a right display associated with a right eye of the user, and displaying the first visual stimulus at the first depth further comprises: concurrently displaying the first visual stimulus at a left position in the left display and displaying the first visual stimulus at a right position in the right display, the left position being distinct from the right position.

Clause 40. The method of any of Clauses 35-39, further comprising: identifying a target depth associated with the depth perception level, the target depth being measured on the first line of sight; and determining the first depth and the second depth based on the target depth, the first depth and the second depth having a first depth resolution.

Clause 41. The method of Clause 40, further comprising, for each of a plurality of depth resolutions: determining a respective pair of depths based on the respective depth resolution and the target depth; and displaying a respective visual stimulus at the respective pair of depths; and obtaining a respective user response; wherein the depth perception level is determined for the user based on both the one or more user responses and the respective user responses corresponding to the plurality of depth resolutions.

Clause 42. The method of Clause 40 or 41, further comprising: scanning a plurality of line depths including the target depth along the first line of sight to determine a respective depth perception level for each line depth on the first line of sight.

Clause 43. The method of Clause 40 or 41, further comprising: scanning a plurality of line depths on each of a plurality of lines of sight to determine a set of depth perception levels for the plurality of line depths on each line of sight, wherein the plurality of line depths are scanned on the first line of sight and include the target depth on the first line of sight.

Clause 44. The method of Clause 43, further comprising forming a depth perception map for the user based on depth perception level sets that are determined for the plurality of line depths on the plurality of lines of sight.

Clause 45. The method of any of Clauses 35-44, wherein determining the depth perception level further comprises: in accordance with a determination that the one or more user responses indicate that the user recognizes that the first depth is different from the second depth, determining that the depth perception level is at least a difference of the first depth and the second depth.

Clause 46. The method of any of Clauses 35-45, wherein displaying the first visual stimulus at the first depth further comprises: rendering a first version of the first visual stimulus on a left display; rendering a second version of the first visual stimulus on a right display, wherein the first version and the second version is different from one another, thereby creating the first depth in the user's eyes.

Clause 47. The method of any of Clauses 35-46, wherein the one or more user responses include a user input captured by one or more first sensors of the electronic device, and the one or more first sensors include a forward-facing camera for detecting a hand gesture and a microphone for collecting an audio response.

Clause 48. The method of any of Clauses 35-47, wherein the one or more user responses include a spontaneous user response monitored by one or more second sensors of the electronic device, and the one or more second sensors include one or more of: an eye tracking camera, a heart rate sensor, a body temperature sensor, a blood oxygen level, a Galvanic skin response sensor, a hand gesture camera, a body gesture camera, a microphone, a motion sensor, and a set of one or more brain activity electrodes.

Clause 49. The method of any of Clauses 35-47, wherein obtaining one or more user responses further includes obtaining a plurality of eye images of the eye while the first stimulus is displayed at the first depth and the second depth, and each eye image corresponds to a respective eye focal length.

Clause 50. The method of Clause 49, further comprising: applying a focus extraction model to process the plurality of eye images and determine two distinct eye focal lengths corresponding to the first depth and the second depth; and automatically and without user intervention, determining whether the eye differentiates the first depth from the second depth based on the two distinct eye focal lengths.

Clause 51. The method of any of Clauses 35-50, wherein a size of the first stimulus is displayed adaptively with the first depth and the second depth.

Clause 52. A method for testing vision, comprising: at an electronic device including a head-mounted display (HMD): identifying a plurality of lines of sight; for each of the plurality of lines of sight: displaying two visual stimuli at two respective depths surrounding each of a plurality of target depth; and obtaining a user response to displaying of the two stimuli at the two depths surrounding each respective target depth; and based on user responses associated with the respective target depths of the plurality of lines of sight, forming a depth perception map for the user associated with the electronic device.

Clause 53. The method of Clause 52, wherein the two visual stimuli are displayed concurrently at the two depths surrounding each respective target depth.

Clause 54. The method of Clause 52 or 53, wherein the two visual stimuli are displayed sequentially at the two depths surrounding each respective target depth.

Clause 55. The method of Clause 52, 53, or 54, further comprising: for each of the plurality of target depths, decreasing a difference of the two depths where the two visual stimuli are displayed until the corresponding user response indicates that the user cannot recognize the difference of the two depths.

Clause 56. A method for testing vision, comprising: at an electronic device including a head-mounted display (HMD): executing a visual assessment application, including generating a user interface corresponding to a three-dimensional (3D) virtual environment; displaying a plurality of visual stimuli in the user interface, each visual stimulus is displayed in duplication with respect to a respective target depth; receiving one or more user responses, each user response indicating whether a user perceives a corresponding visual stimulus in duplication at the respective target depth; and based on the one or more user response, determining a depth perception profile of the user, the depth perception profile including a plurality of depth perception levels corresponding to a plurality of target depths.

Clause 57. The method of Clause 56, wherein each visual stimulus is displayed in duplication at a first position and a second position, and the first position, the second position, and an intermediate position between the first position and the second position are aligned to one another on a respective line of sight, and wherein the intermediate position corresponds to the respective target depth.

Clause 58. The method of Clause 56 or 57, wherein the plurality of visual stimuli are distributed in a binocular area of a field of view of a user associated with the electronic device.

Clause 59. The method of Clause 58, wherein: the binocular area includes a focus area and a peripheral area; a first set of visual stimuli are distributed in the focus area with a first density, and a second set of visual stimuli are distributed in the peripheral area with a second density; and the first density is greater than the second density.

Clause 60. The method of any of Clauses 56-59, wherein the plurality of visual stimuli are displayed concurrently on the user interface.

Clause 61. The method of any of Clauses 56-59, wherein the plurality of visual stimuli are divided into a plurality of groups of visual stimuli, and each group of visual stimuli are displayed concurrently on the user interface, and wherein the plurality of groups of visual stimuli are displayed successively on the user interface.

Clause 62. The method of any of Clauses 56-61, wherein the one or more user responses include a user input captured by one or more first sensors of the electronic device, and the one or more first sensors include a controller for receiving a hand action, a forward-facing camera for detecting a hand gesture, and a microphone for collecting an audio response.

Clause 63. The method of any of Clauses 56-62, wherein the one or more user responses include a spontaneous user response monitored by one or more second sensors of the electronic device, and the one or more second sensors include one or more of: an eye tracking camera, a heart rate sensor, a body temperature sensor, a blood oxygen level, a Galvanic skin response sensor, a hand gesture camera, a body gesture camera, a microphone, a motion sensor, and a set of one or more brain activity electrodes.

Clause 64. The method of any of Clauses 56-62, wherein obtaining one or more user responses further includes obtaining a plurality of eye images of the eye while a first stimulus is displayed at a first depth and a second depth sequentially, and each eye image corresponds to a respective focal length.

Clause 65. The method of Clause 64, further comprising: applying a focus extraction model to process the plurality of eye images and determine two distinct focal lengths corresponding to the first depth and the second depth; and automatically and without user intervention, determining whether the eye differentiates the first depth from the second depth based on the two distinct focal lengths.

Clause 66. The method of any of Clauses 56-65, further comprising: selecting a background view; rendering a static image or a stream of video data associated with the background view on the user interface; and overlaying the plurality of visual stimuli on the static image or a set of respective image frames in the stream of video data associated with the background view.

Clause 67. The method of Clause 66, wherein the background view is one of: a static beach view, a static city night scene, and a dynamic traffic view.

Clause 68. A method for testing vision, comprising: at an electronic device including a head-mounted display (HMD): executing a visual assessment application, including generating a user interface corresponding to a three-dimensional (3D) virtual environment ; displaying a plurality of visual stimuli at a first distance in the 3D virtual environment, the plurality of visual stimuli having a first optotype size and a plurality of first shadings, the first distance and the first optotype size defining a first acuity level; obtaining one or more user responses; based on the one or more user responses, determining a first contrast perception level corresponding to the first acuity level; determining a shading range for a second acuity level based on the first contrast perception level; and determining a plurality of second shadings in the shading range; and displaying the plurality of visual stimuli at a second distance in the 3D virtual environment, the plurality of visual stimuli having a second optotype size and the plurality of second shadings.

Clause 69. The method of Clause 68, further comprising, successively: determining a contrast perception level corresponding to each of the second acuity level and one or more third acuity levels; determining a shading range for each of the one or more third acuity levels; and for each of the one or more third acuity levels, displaying the plurality of visual stimuli at a respective third distance in the 3D virtual environment.

Clause 70. The method of Clause 68 or 69, further comprising: generating a contrast profile of a user associated with the electronic device, the contrast profile mapping the contrast perception level with respect to a plurality of acuity levels including the first acuity level.

Clause 71. The method of Clause 70, wherein the contrast profile includes a plurality of data pairs, and each data pair includes a respective contrast perception level and a respective acuity level, the plurality of data pairs including a first data pair including the first contrast perception level and the first acuity level.

Clause 72. The method of Clause 70 or 71, further comprising: generating a contract sensitivity profile of the user based on the contrast profile.

Clause 73. The method of any of 70-72, further comprising: determining that the user has an eye disease condition based on the contrast profile.

Clause 74. The method of Clause 73, further comprising: obtaining a plurality of reference profiles corresponding to a plurality of known eye conditions; and comparing the contrast profile of the user with each of the plurality of reference profiles to identify the eye disease condition.

Clause 75. The method of Clause 73, further comprising: comparing the contrast profile of the user with one or more reference contrast profiles of the eye disease condition to determine a severity level of the eye disease condition.

Clause 76. The method of Clause 73, further comprising: applying a condition diagnosis model to process the contrast profile to determine that the user has the eye disease condition.

Clause 77. The method of Clause 73 or 76, further comprising: applying a severity diagnosis model to determine a severity level of the eye disease condition.

Clause 78. The method of any of Clauses 68-77, wherein the first optotype size is equal to the second optotype size, and the first distance is different from the second distance.

Clause 79. The method of any of Clauses 68-77, wherein the first optotype size is different from the second optotype size, and the first distance is equal to the second distance.

Clause 80. The method of any of Clauses 68-79, wherein the one or more user responses include a user input captured by one or more first sensors of the electronic device, and the one or more first sensors include a forward-facing camera for detecting a hand gesture and a microphone for collecting an audio response.

Clause 81. The method of any of Clauses 68-80, wherein the one or more user responses include a spontaneous user response monitored by one or more second sensors of the electronic device, and the one or more second sensors include one or more of: an eye tracking camera, a heart rate sensor, a body temperature sensor, a blood oxygen level, a Galvanic skin response sensor, a hand gesture camera, a body gesture camera, a microphone, a motion sensor, and a set of one or more brain activity electrodes.

Clause 82. The method of any of Clauses 68-81, wherein the plurality of first respective shadings corresponds to a first shading resolution, the method further comprising: determining a response time of the one or more user responses; in accordance with a determination that the response time is greater than a response threshold, determine a second shading resolution for the plurality of plurality of second shadings, the second shading resolution is lower than the first shading resolution.

Clause 83. A method for testing vision, comprising: at an electronic device including a head-mounted display (HMD): successively displaying a plurality of visual stimuli corresponding to a plurality of acuity levels in a three-dimensional (3D) virtual environment, wherein at each acuity level, the plurality of visual stimuli have a plurality of respective shadings; obtaining a plurality of user responses to the plurality of visual stimuli; for each acuity level, based on the one or more user responses, determining a respective contrast perception level corresponding to the respective acuity level; and generating a contrast profile of a user associated with the electronic device, the contrast profile mapping the respective contrast perception level with respect to the respective acuity level for the plurality of distances.

Clause 84. The method of Clause 83, wherein: the plurality of acuity levels corresponds to a plurality of distances; at each distance, the plurality of visual stimuli have a respective optotype size, and a respective acuity level is defined based on the respective distance and the respective optotype size.

Clause 85. The method of Clause 83 or 84, further comprising: generating a contract sensitivity profile of the user based on the contrast profile.

Clause 86. The method of any of Clauses 83-85, further comprising: determining that the user has an eye disease condition based on the contrast profile.

Clause 87. The method of Clause 87, further comprising: determining a severity level of the eye disease condition based on the contrast profile or the contrast sensitivity profile.

Clause 88. A method for testing vision, comprising: at an electronic device including a head-mounted display (HMD), one or more processors, and memory: executing a user application configured to enable the vision test; obtaining an instruction to implement a target vision test; in accordance with a determination that the target vision test corresponds to a driver license issuing requirement: loading a VR user interface to create a 3D VR environment; determining an illumination scheme; and displaying a virtual traffic scene on the VR user interface based on the illumination scheme, the virtual traffic scene including a plurality of traffic signs located at a plurality of distances.

Clause 89. The method of Clause 88, wherein each traffic sign is displayed with a set of display parameters including a sign size.

Clause 90. The method of Clause 88 or 89, further comprising displaying a plurality of traffic related objects in the virtual traffic scene, the traffic related objects including one or more of: a traffic light, a pedestrian, and a car.

Clause 91. The method of Clause 90, wherein at least one of the traffic related objects is moving in the virtual traffic scene.

Clause 92. The method of any of Clauses 88-91, wherein the illumination scheme corresponds to a brightness level and a contrast level, and is uniformly applied to the virtual traffic scene.

Clause 93. The method of any of Clauses 88-91, wherein the illumination scheme corresponds to a sun position, the method further comprising: in accordance with the illumination scheme, adaptively rendering the virtual traffic scene based on the sun position.

Clause 94. The method of Clause 93, wherein the sun position includes a solar altitude angle (Alt) and a solar azimuth angle (Az).

Clause 95. The method of any of Clauses 88-94, wherein the illumination scheme corresponds to an ego vehicle headlight, and the illumination scheme is configured to illuminate a local portion of the virtual traffic scene in proximity to a user associated with the electronic device.

Clause 96. The method of Clause 95, wherein at least one of the plurality of traffic signs is exposed to illumination of the ego vehicle headlight.

Clause 97. The method of any of Clauses 88-96, wherein the illumination scheme corresponds to one or more alternative vehicle headlights, and the illumination scheme is configured to illuminate one or more local areas of the virtual traffic scene based on locations of the one or more alternative vehicle headlights.

Clause 98. The method of Clause 97, wherein at least one of the plurality of traffic signs is exposed to illumination of the one or more alternative vehicle headlights.

Clause 99. The method of any of Clauses 88-98, further comprising, while displaying the plurality of traffic signs on the virtual traffic scene: monitoring a user response to each of a subset of traffic signs.

Clause 100. The method of Clause 99, wherein the user response includes a user input captured by one or more first sensors of the electronic device, and the one or more first sensors include a forward-facing camera for detecting a hand gesture and a microphone for collecting an audio response.

Clause 101. The method of Clause 99 or 100, wherein the user response includes a spontaneous user response monitored by one or more second sensors of the electronic device, and the one or more second sensors include one or more of: an eye tracking camera, a heart rate sensor, a body temperature sensor, a blood oxygen level, a Galvanic skin response sensor, a hand gesture camera, a body gesture camera, a microphone, a motion sensor, and a set of one or more brain activity electrodes.

Clause 102. The method of Clause 101, further comprising: determining a response time of the user response associated with a first traffic sign; and in accordance with a determination that the response time is greater than a response threshold, adjusting the illumination scheme to update the plurality of traffic signs on the virtual traffic scene.

Clause 103. The method of Clause 101 or 102, further comprising: determining a current success rate for the subset of traffic signs; and in accordance with a determination that the current success rate is lower than a failure threshold, adjusting the illumination scheme to update the plurality of traffic signs on the virtual traffic scene.

Clause 104. A method for implementing a vision test, comprising: at an electronic device including a head-mounted display (HMD): executing a user application configured to enable the vision test; generating a user interface corresponding to a three-dimensional (3D) virtual environment; displaying a plurality of traffic signs at a plurality of distances on a virtual traffic scene; and applying an illumination scheme to the virtual traffic scene.

Clause 105. The method of Clause 104, wherein the illumination scheme corresponds to a brightness level and a contrast level, and is uniformly applied to the virtual traffic scene.

Clause 106. The method of Clause 104, wherein the illumination scheme corresponds to a sun position, and the illumination scheme is configured to illuminate the virtual traffic scene based on the sun position.

Clause 107. The method of Clause 106, wherein the sun position includes a solar altitude angle (Alt) and a solar azimuth angle (Az).

Clause 108. The method of any of Clauses 104-107, wherein the illumination scheme corresponds to an ego vehicle headlight, and the illumination scheme is configured to illuminate a local portion of the virtual traffic scene in proximity to a user associated with the electronic device, and at least one of the plurality of traffic signs is exposed to illumination of the ego vehicle headlight.

Clause 109. The method of any of Clauses 104-107, wherein the illumination scheme corresponds to one or more alternative vehicle headlights, and the illumination scheme is configured to illuminate one or more local areas of the virtual traffic scene based on locations of the one or more alternative vehicle headlights, and at least one of the plurality of traffic signs is exposed to illumination of the one or more alternative vehicle headlights.

Clause 110. A method for testing vision, comprising: at an electronic device including a head-mounted display (HMD): executing a visual assessment application, including generating a user interface corresponding to a three-dimensional (3D) virtual environment; identifying a first line of sight of a user associated with the electronic device; selecting a plurality of positions on the first line of sight, wherein each position is located in a respective position range; for each position, displaying an object at a plurality of locations within the respective position range; obtaining a plurality of user responses to displaying the object for each position; and based on the plurality of user responses, determining a depth perception level of the user associated with the first line of sight.

Clause 111. The method of Clause 110, further comprising: identifying a standard line of sight that extends forward from a center of, and is perpendicular to, a line connecting two eyes of a user, wherein the first line of sight has a first angle with respect to the standard line of sight.

Clause 112. The method of Clause 111, wherein the first line of sight is the standard line of sight, and the first angle is equal to 0.

Clause 113. The method of Clause 111, wherein the first angle is within a binocular angular range, the method further comprising: in accordance with a determination that the depth perception level is lower than a reference depth level, determining that the user's depth perception is compromised.

Clause 114. The method of Clause 110, wherein the depth perception level includes a plurality of depth sensitivity levels, and the method further comprises, for each position on the first line of sight, determining a respective depth sensitivity level based on a subset of user responses.

Clause 115. The method of Clause 114, further comprising, for each position: based on the plurality of user responses, identifying two of the plurality of locations that are differentiated by the user and have a distance that is smaller than distances between any other two of the plurality of locations, wherein the respective sensitivity level includes the distance of the two of the plurality of locations.

Clause 116. The method of any of Clauses 110-115, further comprising: identifying one or more second lines of sight; for each second line of sight, determining a respective depth perception level, thereby forming a depth perception map associating the first line of sight and the one or more second lines of sight with the depth perception levels.

Clause 117. The method of Clause 116, further comprising: generating a depth heatmap indicating a region having a depth perception level within a normal range.

Clause 118. The method of Clause 116 or 117, determining the respective depth perception level for each second line of sight further comprising: selecting a plurality of second positions on the second line of sight, wherein each second position is located in a respective second position range; for each second position, displaying the object at a plurality of second locations within the respective second position range; and obtaining a plurality of second user responses to displaying the object for each second position, wherein based on the plurality of second user responses, determining the depth perception level of the user associated with the second line of sight.

Clause 119. The method of Clause any of Clauses 116-118, further comprising: comparing the depth perception map with a reference perception map to determine whether the user's depth perception is compromised.

Clause 120. The method of Clause 119, further comprising: determining a depth perception range of the user based on the depth perception map; and comparing the depth perception range with a predefined binocular angular range.

Clause 121. The method of Clause 120, further comprising: in accordance with a determination that the depth perception range includes the predefined binocular angular range, determining that the depth perception level of the user is proper.

Clause 122. The method of Clause 120, further comprising: in accordance with a determination that the depth perception range misses a subset of the predefined binocular angular range, determining that the depth perception level of the user is compromised.

Clause 123. The method of any of Clauses 110-122, wherein the plurality of user responses include a user input captured by one or more first sensors of the electronic device, and the one or more first sensors include a forward-facing camera for detecting a hand gesture and a microphone for collecting an audio response.

Clause 124. The method of any of Clauses 110-123, wherein the plurality of user responses include a spontaneous user response monitored by one or more second sensors of the electronic device, and the one or more second sensors include one or more of: an eye tracking camera, a heart rate sensor, a body temperature sensor, a blood oxygen level, a Galvanic skin response sensor, a hand gesture camera, a body gesture camera, a microphone, a motion sensor, and a set of one or more brain activity electrodes.

Clause 125. The method of Clause 124, further comprising: determining a response time associated with displaying of the object; and adjusting the depth perception level of the user based on the response time.

Clause 126. The method of Clause 124 or 125, further comprising: determining a current success rate associated with displaying of the object; and adjusting the depth perception level of the user based on the current success rate.

Clause 127. A method for testing vision, comprising: at an electronic device including a head-mounted display (HMD): executing a visual assessment application, including generating a user interface corresponding to a three-dimensional (3D) virtual environment; identifying a plurality of lines of sight of a user associated with the electronic device; generating a depth perception map associated with the plurality of lines of sight; and determining a depth perception range of the user based on the depth perception map.

Clause 128. The method of Clause 127, further comprising: identifying a standard line of sight that extends forward from a center of, and is perpendicular to, a line connecting two eyes of a user, wherein each line of sight has a respective angle with respect to the standard line of sight.

Clause 129. The method of Clause 127 or 128, wherein the depth perception map includes a plurality of depth perception levels corresponding to a plurality of positions on at least one of the plurality of lines of sight.

Clause 130. An interactive virtual-reality method for performing a virtual vision test and displaying media, as discussed in any of Clauses 1-129.

Clause 131. A non-transitory computer readable storage medium, storing one or more programs for execution by one or more processors of a computer system, the one or more programs including instructions for implementing a method in any of Clauses 1-129.

Clause 132. A computer system, comprising: one or more processors; and memory for storing one or more programs for execution by the one or more processors, the one or more programs including instructions for implementing a method in any of Clauses 1-129.

In some embodiments, any of the above clauses herein may depend from any one of the independent clauses or any one of the dependent clauses. In one aspect, any of the clauses (e.g., dependent or independent clauses) may be combined with any other one or more clauses (e.g., dependent or independent clauses). In one aspect, a claim may include some or all of the words (e.g., steps, operations, means or components) recited in a clause, a sentence, a phrase or a paragraph. In one aspect, a claim may include some or all of the words recited in one or more clauses, sentences, phrases or paragraphs. In one aspect, some of the words in each of the clauses, sentences, phrases or paragraphs may be removed. In one aspect, additional words or elements may be added to a clause, a sentence, a phrase or a paragraph. In one aspect, the subject technology may be implemented without utilizing some of the components, elements, functions or operations described herein. In one aspect, the subject technology may be implemented utilizing additional components, elements, functions or operations.

### Further Considerations

As used herein, the word module refers to logic embodied in hardware or firmware, or to a collection of software instructions, possibly having entry and exit points, written in a programming language, such as, for example C++. A software module may be compiled and linked into an executable program, installed in a dynamic link library, or may be written in an interpretive language such as BASIC. It will be appreciated that software modules may be callable from other modules or from themselves, and/or may be invoked in response to detected events or interrupts. Software instructions may be embedded in firmware, such as an EPROM or EEPROM. It will be further appreciated that hardware modules may be comprised of connected logic units, such as gates and flip-flops, and/or may be comprised of programmable units, such as programmable gate arrays or processors. The modules described herein are preferably implemented as software modules, but may be represented in hardware or firmware.

It is contemplated that the modules may be integrated into a fewer number of modules. One module may also be separated into multiple modules. The described modules may be implemented as hardware, software, firmware or any combination thereof. Additionally, the described modules may reside at different locations connected through a wired or wireless network, or the Internet.

In general, it will be appreciated that the processors can include, by way of example, computers, program logic, or other substrate configurations representing data and instructions, which operate as described herein. In other embodiments, the processors can include controller circuitry, processor circuitry, processors, general purpose single-chip or multi-chip microprocessors, digital signal processors, embedded microprocessors, microcontrollers and the like.

Furthermore, it will be appreciated that in one embodiment, the program logic may advantageously be implemented as one or more components. The components may advantageously be configured to execute on one or more processors. The components include, but are not limited to, software or hardware components, modules such as software modules, object-oriented software components, class components and task components, processes methods, functions, attributes, procedures, subroutines, segments of program code, drivers, firmware, microcode, circuitry, data, databases, data structures, tables, arrays, and variables.

The foregoing description is provided to enable a person skilled in the art to practice the various configurations described herein. While the subject technology has been particularly described with reference to the various figures and configurations, it should be understood that these are for illustration purposes only and should not be taken as limiting the scope of the subject technology.

There may be many other ways to implement the subject technology. Various functions and elements described herein may be partitioned differently from those shown without departing from the scope of the subject technology. Various modifications to these configurations will be readily apparent to those skilled in the art, and generic principles defined herein may be applied to other configurations. Thus, many changes and modifications may be made to the subject technology, by one having ordinary skill in the art, without departing from the scope of the subject technology.

It is understood that the specific order or hierarchy of steps in the processes disclosed is an illustration of exemplary approaches. Based upon design preferences, it is understood that the specific order or hierarchy of steps in the processes may be rearranged. Some of the steps may be performed simultaneously. The accompanying method claims present elements of the various steps in a sample order, and are not meant to be limited to the specific order or hierarchy presented.

As used herein, the phrase at least one of preceding a series of items, with the term and or or to separate any of the items, modifies the list as a whole, rather than each member of the list (i.e., each item). The phrase at least one of does not require selection of at least one of each item listed; rather, the phrase allows a meaning that includes at least one of any one of the items, and/or at least one of any combination of the items, and/or at least one of each of the items. By way of example, the phrases at least one of A, B, and C or at least one of A, B, or C each refer to only A, only B, or only C; any combination of A, B, and C; and/or at least one of each of A, B, and C.

Terms such as top, bottom, front, rear and the like as used in this disclosure should be understood as referring to an arbitrary frame of reference, rather than to the ordinary gravitational frame of reference. Thus, a top surface, a bottom surface, a front surface, and a rear surface may extend upwardly, downwardly, diagonally, or horizontally in a gravitational frame of reference.

Furthermore, to the extent that the term include, have, or the like is used in the description or the claims, such term is intended to be inclusive in a manner similar to the term comprise as comprise is interpreted when employed as a transitional word in a claim.

As used herein, the term about is relative to the actual value stated, as will be appreciated by those of skill in the art, and allows for approximations, inaccuracies and limits of measurement under the relevant circumstances. In one or more aspects, the terms about, substantially, and approximately may provide an industry-accepted tolerance for their corresponding terms and/or relativity between items.

As used herein, the term comprising indicates the presence of the specified integer(s), but allows for the possibility of other integers, unspecified. This term does not imply any particular proportion of the specified integers. Variations of the word comprising, such as comprise and comprises, have correspondingly similar meanings.

The word exemplary is used herein to mean serving as an example, instance, or illustration. Any embodiment described herein as exemplary is not necessarily to be construed as preferred or advantageous over other embodiments.

A reference to an element in the singular is not intended to mean one and only one unless specifically stated, but rather one or more. Pronouns in the masculine (e.g., his) include the feminine and neuter gender (e.g., her and its) and vice versa. The term some refers to one or more. Underlined and/or italicized headings and subheadings are used for convenience only, do not limit the subject technology, and are not referred to in connection with the interpretation of the description of the subject technology. All structural and functional equivalents to the elements of the various configurations described throughout this disclosure that are known or later come to be known to those of ordinary skill in the art are expressly incorporated herein by reference and intended to be encompassed by the subject technology. Moreover, nothing disclosed herein is intended to be dedicated to the public regardless of whether such disclosure is explicitly recited in the above description.

Although the detailed description contains many specifics, these should not be construed as limiting the scope of the subject technology but merely as illustrating different examples and aspects of the subject technology. It should be appreciated that the scope of the subject technology includes other embodiments not discussed in detail above. Various other modifications, changes and variations may be made in the arrangement, operation and details of the method and apparatus of the subject technology disclosed herein without departing from the scope. In addition, it is not necessary for a device or method to address every problem that is solvable (or possess every advantage that is achievable) by different embodiments of the disclosure in order to be encompassed within the scope of the disclosure. The use herein of can and derivatives thereof shall be understood in the sense of possibly or optionally as opposed to an affirmative capability.

What is claimed is:

1. A method for testing vision, comprising:
at an electronic device including a head-mounted display (HMD):
executing a visual assessment application, including generating a user interface corresponding to a three-dimensional (3D) virtual environment;
displaying a visual stimulus at a target location in the 3D virtual environment;
monitoring a head orientation of a user wearing the electronic device; and
dynamically adjusting the target location of the visual stimulus based on the head orientation.

2. The method of claim 1, further comprising:
identifying a standard line of sight that extends forward from a center of, and is perpendicular to, a line connecting two eyes of the user; and
selecting the target location on the standard line of sight.

3. The method of claim 1, wherein the target location corresponds to a first orientation, and dynamically adjusting the target location of the visual stimulus further comprises:
determining that the head orientation has stabilized at a current orientation distinct from the first orientation for a first extended duration of time; and
in accordance with a determination with the first extended duration of time is greater than an orientation threshold, moving the target location to follow the current orientation.

4. The method of claim 1, further comprising:
monitoring an eye position based on eye images captured by an eye-tracking camera, wherein the target location of the visual stimulus is dynamically adjusted based on both the head orientation and the eye position.

5. The method of claim 4, wherein the target location corresponds to a first line of sight, the method further comprising:
determining that the eye position has stabilized along a current line of sight distinct from the first line of sight for a second extended duration of time; and
in accordance with a determination with the second extended duration of time is greater than a sight line threshold, moving the target location to follow the current line of sight.

6. The method of claim 1, further comprising:
obtaining a motion signal measured by a motion sensor, wherein the motion sensor includes one or more of: an accelerometer and a gyroscope, and the head orientation is determined based on the motion signal.

7. The method of claim 1, further comprising:
receiving a user response indicating whether the visual stimulus is clear to the user, wherein the user response includes a user input captured by one or more first sensors of the electronic device, and the one or more first sensors include a forward facing camera for detecting a hand gesture, a microphone for collecting an audio response, and a controller for receiving a user physical force.

8. The method of claim 1, further comprising:
receiving a user response indicating whether the visual stimulus is clear to the user, wherein the user response include a spontaneous user response monitored by one or more second sensors of the electronic device, and the one or more second sensors include one or more of: an eye tracking camera, a heart rate sensor, a body temperature sensor, a blood oxygen level, a Galvanic skin response sensor, a hand gesture camera, a body gesture camera, a microphone, a motion sensor, and a set of one or more brain activity electrodes; and
based on the user response, automatically, determining whether the visual stimulus is clear to the user.

9. The method of claim 1, wherein the visual stimulus has a first stimulus size at the target location, the method further comprising:
while keeping the first stimulus size, displaying the visual stimulus at one or more alternative locations that are different from the target location; and
receiving a user response indicating that the visual stimulus starts to be clear at the target location to the user compared with the one or more alternative locations.

10. The method of claim 1, wherein the visual stimulus has a first stimulus size at the target location, further comprising:
while displaying the visual stimulus at the target location, varying a size of the visual stimulus to one or more alternative stimulus sizes; and
receiving a user response indicating that the visual stimulus starts to be clear at the first stimulus size to the user compared with the one or more alternative stimulus sizes.

11. A non-transitory computer readable storage medium, storing one or more programs for execution by one or more processors of an electronic device including an HMD, the one or more programs including instructions for:
executing a visual assessment application, including generating a user interface corresponding to a three-dimensional (3D) virtual environment;
displaying a visual stimulus at a target location in the 3D virtual environment;
monitoring a head orientation of a user wearing the electronic device; and
dynamically adjusting the target location of the visual stimulus based on the head orientation.

12. The non-transitory computer readable storage medium of claim 11, wherein the visual stimulus has a first stimulus size at the target location, the one or more programs further comprising instructions for:
determining a target distance between the target location and the user; and
determining a spherical power based on the target distance and the first stimulus size.

13. The non-transitory computer readable storage medium of claim 11, the one or more programs further comprising instructions for:
identifying a standard line of sight that extends forward from a center of, and is perpendicular to, a line connecting two eyes of the user; and
selecting the target location on the standard line of sight.

14. The non-transitory computer readable storage medium of claim 11, wherein the target location corresponds to a first orientation, and dynamically adjusting the target location of the visual stimulus further comprises:
determining that the head orientation has stabilized at a current orientation distinct from the first orientation for a first extended duration of time; and
in accordance with a determination with the first extended duration of time is greater than an orientation threshold, moving the target location to follow the current orientation.

15. The non-transitory computer readable storage medium of claim 11, the one or more programs further comprising instructions for:
monitoring an eye position based on eye images captured by an eye-tracking camera, wherein the target location of the visual stimulus is dynamically adjusted based on both the head orientation and the eye position.

16. The non-transitory computer readable storage medium of claim 15, wherein the target location corresponds to a first line of sight, the one or more programs further comprising instructions for:
determining that the eye position has stabilized along a current line of sight distinct from the first line of sight for a second extended duration of time; and
in accordance with a determination with the second extended duration of time is greater than a sight line threshold, moving the target location to follow the current line of sight.

17. An electronic device, comprising:
an HMD;
one or more processors; and
memory for storing one or more programs for execution by the one or more processors, the one or more programs including instructions for:
executing a visual assessment application, including generating a user interface corresponding to a three-dimensional (3D) virtual environment;
displaying a visual stimulus at a target location in the 3D virtual environment;
monitoring a head orientation of a user wearing the electronic device; and
dynamically adjusting the target location of the visual stimulus based on the head orientation.

18. The electronic device of claim 17, the one or more programs further comprising instructions for:
obtaining a motion signal measured by a motion sensor, wherein the motion sensor includes one or more of: an accelerometer and a gyroscope, and the head orientation is determined based on the motion signal.

19. The electronic device of claim 17, wherein the visual stimulus has a first stimulus size at the target location, he one or more programs further comprising instructions for:
while keeping the first stimulus size, displaying the visual stimulus at one or more alternative locations that are different from the target location; and
receiving a user response indicating that the visual stimulus starts to be clear at the target location to the user compared with the one or more alternative locations.

20. The electronic device of claim 17, wherein the visual stimulus has a first stimulus size at the target location, he one or more programs further comprising instructions for:
while displaying the visual stimulus at the target location, varying a size of the visual stimulus to one or more alternative stimulus sizes; and
receiving a user response indicating that the visual stimulus starts to be clear at the first stimulus size to the user compared with the one or more alternative stimulus sizes.
